(19) United States
(12) Patent Application Publication
(10) Pub. No.: US 2020/0378061 A1
(43) Pub. Date: Dec. 3, 2020

(54) COMPOSITIONS, TREATING METHODS, AND TREATED FIBROUS SUBSTRATES

(71) Applicant: 3M INNOVATIVE PROPERTIES COMPANY, St. Paul, MN (US)

(72) Inventors: Rudolf J. Dams, Antwerp (BE); Dirk M. Coppens, Beveren (BE)

(21) Appl. No.: 16/756,276

(22) PCT Filed: Nov. 26, 2018
(86) PCT No.: PCT/US2018/062444

§ 371 Date: Apr. 15, 2020

Related U.S. Application Data
(60) Provisional application No. 62/592,506, filed on Nov. 30, 2017.

(51) Int. Cl. D06M 15/263; C07C 57/04; C07C 233/05; C09D 175/16; C09D 133/10; C09D 133/26; D06M 15/564; D06M 15/285; D06M 15/267

(52) U.S. Cl. CPC D06M 15/263; C07C 57/04; C07C 233/05; C09D 175/16; C09D 133/10; D06M 2101/34; D06M 15/564; D06M 15/285; D06M 15/267; D06M 2200/10; C09D 133/26

(57) ABSTRACT

Fluorine free compositions for treating fibrous substrates to make the fibrous substrates water repellent, method for treating the fibrous substrates with said compositions and water repellent fibrous substrates treated with said fluorine free compositions.

### BACKGROUND

Compositions for treating fibrous substrates to enhance the water-repellency of the substrates are known and described in the literature, for example, in “Fluorinated Surfactants and Repellents,” E. Kissa, Surfactant Science Series, vol. 97, Marcel Dekker, New York, Chapter 12, p. 516-551, or in “Chemistry and Technology of Silicones,” by W. Noll, Academic Press, New York, Chapter 10, p. 585-595. Substrates that are treated with fluorinated repellents typically have high durable water repellency properties. Fluorinated treating agents, however, suffer from some disadvantages, including their production price, and they may be subject to some potential environmental concerns. In recent years, fluorine-free alternative repellents have received increased interest. Such repellents may provide good initial water repellency to fibrous substrates, but typically durability is not satisfactory. Accordingly, there is a continual need for compositions that provide high water repellency, in particular high initial water repellency, and in certain situations, high water-repellent durability, especially compositions that are fluorine-free.

### SUMMARY OF THE DISCLOSURE

The present disclosure provides compositions, methods, and treated substrates.

In a first embodiment, a method of treating a fibrous substrate is provided. The method includes applying a fluorine-free treating composition in an amount sufficient to make the fibrous substrate water repellent, wherein the treating composition includes one or more polymeric compounds derived from the polymerization of at least one monomer of Formula (I):

R¹-D¹-C(O)C(R²)═CH₂ Formula (I)

wherein:

R¹ represents a hydrocarbon group having 4 to 60 carbon atoms; and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms.

In a second embodiment, a fluorine-free treating composition is provided that includes Component (A) and at least one of Component (B) and Component (C). Component (A) includes one or more polymeric compounds derived from the polymerization of at least one monomer of Formula (T):

R¹-D¹-C(O)C(R²)═CH₂ Formula (I)

wherein:

R¹ represents a hydrocarbon group having 4 to 60 carbon atoms; and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—,
—NHC(O)-L⁶-O—;
—NC(O)-L⁷-C(O)O-L⁸-O—;
—C(O)—N—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms.

In this second embodiment, Component (B) includes one or more compounds derived from reaction, in one or more steps, of components including:
(i) at least one:
   isocyanate-reactive oligomer comprising 2 to 20 repeating units; or
   an alcohol, amine, acid, amide, or thiol, having at least one linear or branched hydrocarbon group having from 4 to 60 carbon atoms and optionally one or more ester groups;
(ii) at least one polyisocyanate;
(iii) optionally at least one additional mono-, di-, or poly-functional isocyanate-reactive compound; and
(iv) optionally at least one isocyanate blocking agent;

wherein the isocyanate-reactive oligomer is made by the radical-initiated reaction of at least one (meth)acrylate or (meth)acrylamide monomer, in the presence of at least one mercaptan, wherein the at least one (meth)acrylate or (meth)acrylamide monomer comprises at least one hydrocarbon group having from 4 to 60 carbon atoms. In this second embodiment, Component (C) comprises one or more polycarbodiimide compounds.

In a third embodiment, a composition is provided that includes Component (B), wherein:

Component (B) comprises one or more compounds derived from reaction, carried out in one or more steps, of components comprising:
(i) at least one:
   isocyanate-reactive oligomer comprising 2 to 20 repeating units; or
   an amine, acid, or amide, having at least one linear or branched hydrocarbon group having from 4 to 60 carbon atoms and optionally one or more ester groups;
(ii) at least one polyisocyanate;
(iii) optionally at least one additional mono-, di- or poly-functional isocyanate-reactive compound; and
(iv) optionally at least one isocyanate blocking agent;

with the proviso that less than 40% of the isocyanate groups are reacted with acid and/or amide groups;

wherein the isocyanate-reactive oligomer has the following formula:

Y¹—[CH₂—C(R²)C(O)-D¹-R¹]ₘ—S—R¹⁰-(T¹)ₚ Formula (V)
wherein:
   Y¹ is H or an initiator residue;
   R¹ is a hydrocarbon group having 4 to 60 carbon atoms;
   R² is independently H or CH₃—;
   D¹ is selected from:
      —C(O)O-L¹-O—;
      —OC(O)-L²-O—;
      —OC(O)-L³-C(O)O-L⁴-O—;
      —C(O)NH-L⁵-O—;
      —NHC(O)-L⁶-O—;
      —NHC(O)-L⁷-C(O)O-L⁸-O—;
      C(O)—NH—;
      —C(O)NHC(O)NH-L⁹-O—;
      —C(O)NHC(O)-L¹⁰-O—;
      —NHC(O)NH—; and
      —NH—; with the proviso that when D¹ is —NH—, the isocyanate-reactive oligomer comprises greater than 30 wt-% of monomeric units that include —NH—, based on the total weight of monomeric units;
   each L¹, L², L³, L⁴, L⁵, L⁶, L⁷, L⁸, L⁹, and L¹⁰ is independently a branched or straight chain alkylene group having 2 to 10 carbon atoms;
      S is sulfur;
      R¹⁰ is a divalent or trivalent linking group having 1 to 10 carbon atoms;
      T¹ is —C(O)OH, —C(O)NH₂, —OH, —NH₂, or —NH(R¹¹), wherein R¹¹ is H or a hydrocarbon group having 1 to 10 carbon atoms;
      m is an integer of 2 to 20; and
      p is independently 1 or 2;
         and/or

Component (B) comprises one or more compounds derived from reaction, carried out in one or more steps, of components comprising:
(i) at least one:
   isocyanate-reactive oligomer comprising 2 to 20 repeating units; or
   an amine, acid, or amide, having at least one linear or branched hydrocarbon group having from 4 to 60 carbon atoms and optionally one or more ester groups
(ii) at least one polyisocyanate;
(iii) optionally at least one additional mono-, di- or poly-functional isocyanate-reactive compound; and
(iv) optionally at least one isocyanate blocking agent;

wherein the isocyanate-reactive oligomer has the following formula:

Y²—[CH₂—C(R⁴)C(O)-D²-R³]ₘ—S—R¹²-(T²)ₚ Formula (VI)
wherein:
   Y² is H or an initiator residue;
   R³ is a hydrocarbon group having 4 to 60 carbon atoms;
   R⁴ is independently H or CH₃;
   D² is selected from:
      —NHC(O)OL¹¹-O—;
      —O—C(O)NH-L¹²-O—;
      —NHC(O)NH-L¹³-O—; and
      —O—; and
   each L¹¹, L¹², and L¹³ is independently a branched or straight chain alkylene group having 2 to 10 carbon atoms;
   S is sulfur;
   R¹² is a divalent or trivalent linking group having 1 to 10 carbon atoms;
   T² is —C(O)OH, —C(O)NH₂, —OH, —NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
   m is an integer of 2 to 20; and
   p is independently 1 or 2.

In a fourth embodiment, a composition is provided that includes Component (C), wherein:

Component (C) comprises at least one polycarbodiimide compound derived from a carbodiimidization reaction, carried out in one or more steps, of components comprising:
(i) at least one:
   isocyanate-reactive oligomer comprising 2 to 20 repeating units; or
   an amine, acid, or amide, having at least one linear or branched hydrocarbon group having from 4 to 60 carbon atoms and optionally one or more ester groups;
(ii) at least one polyisocyanate; and
(iii) optionally at least one additional mono-, di- or poly-functional isocyanate-reactive compound;
wherein the isocyanate-reactive oligomer has the following formula:

Y¹—[CH₂—C(R²)C(O)-D¹-R¹]ₘ—S—R¹⁰-(T¹)ₚ Formula (V)
wherein:
   Y¹ is H or an initiator residue;
   R¹ is a hydrocarbon group having 4 to 60 carbon atoms;
   R² is independently H or CH₃;
   D¹ is selected from:
      —C(O)O-L¹-O—;
      —OC(O)-L²-O—;
      —OC(O)-L³-C(O)O-L⁴-O—;
      —C(O)NH-L⁵-O—;
      —NHC(O)-L⁶-O—;
      —NHC(O)-L-C(O)O-L⁸-O—;
      —C(O)—NH—;
      —C(O)NHC(O)NH—R⁹—O—;
      —C(O)NHC(O)-L¹⁰-O;
      —NHC(O)NH—; and
      —NH—; with the proviso that when D¹ is —N—, the isocyanate-reactive oligomer comprises greater than 30 wt-% of monomeric units that include —NH—, based on the total weight of monomeric units;
   each L¹, L², L³, L⁴, L⁵, L⁶, L⁷, L⁸, L⁹, and L¹⁰ is independently a branched or straight chain alkylene group having 2 to 10 carbon atoms;
   S is sulfur;
   R¹⁰ is a divalent or trivalent linking group having 1 to 10 carbon atoms;
   T¹ is —C(O)OH, —C(O)NH₂, —OH, —NH₂, or —NH(R¹¹), wherein R¹¹ is H or a hydrocarbon group having 1 to 10 carbon atoms;
   m is an integer of 2 to 20; and
   p is independently 1 or 2;
      and/or

Component (C) comprises at least one polycarbodiimide compound derived from a carbodiimidization reaction, carried out in one or more steps, of components comprising:
(i) at least one:
   isocyanate-reactive oligomer comprising 2 to 20 repeating units; or
   an amine, acid, or amide, having at least one linear or branched hydrocarbon group having from 4 to 60 carbon atoms and optionally one or more ester groups;
(ii) at least one polyisocyanate; and
(iii) optionally at least one additional mono-, di- or poly-functional isocyanate-reactive compound;

wherein the isocyanate-reactive oligomer has the following formula:

Y²—[CH₂—C(R⁴)C(O)-D²-R³]ₘ—S—R¹²-(T²)ₚ Formula (VI)
wherein:
   Y² is H or an initiator residue;
   R³ is a hydrocarbon group having 4 to 60 carbon atoms;
   R⁴ is independently H or CH₃;
   D² is selected from:
      —NHC(O)OL¹¹-O—;
      —O—C(O)NH-L¹²-O—;
      —NHC(O)NH-L¹³-O—; and
      —O—; and
   each L¹¹, L¹², and L¹³ is independently a branched or straight chain alkylene group having 2 to 10 carbon atoms;
   S is sulfur;
   R⁷ is a divalent or trivalent linking group having 1 to 10 carbon atoms;
   T² is —C(O)OH, —C(O)NH₂, —OH, NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
   m is an integer of 2 to 20; and
   p is independently 1 or 2.

In a fifth embodiment, the present disclosure provides a method of treating a fibrous substrate. The method includes applying a composition as described herein in an amount sufficient to make the fibrous substrate water repellent.

In a sixth embodiment, the present disclosure provides a fibrous substrate treated by a method as described herein.

Herein, a “fluorine-free” treating composition means that a treating composition includes less than 1 weight percent (1 wt-%) fluorine in a treating composition based on solids, whether in a concentrate or ready-to-use treating composition. In certain embodiments, a “fluorine-free” treating composition means that a treating composition includes less than 0.5 wt %, or less than 0.1 wt %, or less than 0.01 wt-%, fluorine. The fluorine may be in the form of organic or inorganic fluorine-containing compounds.

The term “oligomer” includes compounds with at least 2 repeating units and up to 20 repeating units. According to a particular embodiment, the oligomer has 3 to 15 repeating units. According to another embodiment, the oligomer has 4 to 15 repeating units. In certain embodiments, an “oligomer” has a weight average molecular weight of up to 20,000 Daltons.

The term “polymeric compound” includes compounds with greater than 20 repeating units. In certain embodiments, a “polymeric compound” has a weight average molecular weight of greater than 20,000 Daltons.

The term “residue” means that part of the original organic molecule is remaining after reaction.

The term “hydrocarbon” refers to any substantially fluorine-free organic group that contains hydrogen and carbon. Such hydrocarbon groups may be cyclic (including aromatic), linear, or branched. Suitable hydrocarbon groups include alkyl groups, alkylene groups, arylene groups, and the like. Unless otherwise indicated, hydrocarbon groups typically contain from to 60 carbon atoms. In some embodiments, hydrocarbon groups contain 4 to 60 carbon atoms, 12 to 60 carbon atoms, 12 to 50 carbon atoms, 12 to 40 carbon atoms, 12 to 30 carbon atoms, 16 to 60 carbon atoms, 16 to 50 carbon atoms, 16 to 40 carbon atoms, or 16 to 30 carbon atoms. In some embodiments, hydrocarbon groups contain 1 to 30 carbon atoms, 1 to 20 carbon atoms, 1 to 10 carbon atoms, 1 to 6 carbon atoms, 1 to 4 carbon atoms, or 1 to 3 carbon atoms.

The term “alkyl” refers to a monovalent group that is a residue of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 60 carbon atoms. In some embodiments, the alkyl groups contain 1 to 30 carbon atoms, 1 to 20 carbon atoms, 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, octadecyl (also referred to as stearyl), docosanyl (also referred to as behenyl), adamantyl, norbornyl, and the like.

The term “alkylene” refers to a divalent group that is a residue of an alkane and includes groups that are linear, branched, cyclic, bicyclic, or a combination thereof. Unless otherwise indicated, the alkylene group typically has 1 to 60 carbon atoms. In some embodiments, the alkylene group has 1 to 30 carbon atoms, 1 to 20 carbon atoms, 1 to 10 carbon atoms, 2 to 10 carbon atoms, 1 to 6 carbon atoms, or 1 to 4 carbon atoms. Examples of “alkylene” groups include methylene, ethylene, 1,3-propylene, 1,2-propylene, 1,4-butylene, 1,4-cyclohexylene, 1,6-hexamethylene, and 1,10-decamethylene.

The term “arylene” refers to a divalent group that is aromatic and, optionally, carbocyclic. The arylene has at least one aromatic ring. Optionally, the aromatic ring can have one or more additional carbocyclic rings that are fused to the aromatic ring. Any additional rings can be unsaturated, partially saturated, or saturated. Unless otherwise specified, arylene groups often have 5 to 20 carbon atoms, 5 to 18 carbon atoms, 5 to 16 carbon atoms, 5 to 12 carbon atoms, 6 to 12 carbon atoms, or 6 to 10 carbon atoms.

The term (meth)acrylate refers to acrylates and methacrylates.

The term “polyisocyanate” refers to aliphatic and/or aromatic di-, tri-, and higher polyisocyanates.

The term “isocyanate-reactive” refers to a compound or oligomer that includes a group that is capable of reacting with an isocyanate (i.e., an isocyanate-reactive group such as an alcohol, amine, or amide).

The term “comprises” and variations thereof do not have a limiting meaning where these tens appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an.” and “the” are used interchangeably with the phrases “at least one” and “one or more” The phrases “at least one of” and “comprises at least one of” followed by a list refers to any one of the items in the list and any combination of two or more items in the list.

The term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

The term “room temperature” refers to a temperature of 20° C. to 25° C. or 22° C. to 25° C.

Herein, when a group is present more than once in a formula described herein, each group is “independently” selected, whether specifically stated or not. For example, when more than one Q group is present in a formula, each Q group is independently selected. Furthermore, subgroups contained within these groups are also independently selected.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### DETAILED DESCRIPTION OF ILLUSTRATIVE EMBODIMENTS

The present disclosure provides compositions (e.g., treating compositions, preferably fluorine-free treating compositions for fibrous substrates), methods (e.g., treating methods), and treated fibrous substrates.

Compositions (e.g., treating compositions) of the present disclosure are useful for treating a fibrous substrate to enhance the substrate's water repellency. In certain embodiments, such treating compositions can be used in a method of treating a fibrous substrate. One method may include applying a composition (preferably, a fluorine-free treating composition) in an amount sufficient to make the fibrous substrate water repellent. One method may include applying a composition (preferably, a fluorine-free treating composition) in an amount sufficient to make the fibrous substrate durably water repellent.

As used herein, a fibrous substrate is water repellent if it demonstrates a minimum initial spray rating of at least 80, as determined by the Spray Rating Test described in the Examples Section. In certain embodiments, the initial spray rating is at least 90, or at least 100, as determined by the Spray Rating Test described in the Examples Section.

In certain embodiments, the fibrous substrates are treated such that they become durably water repellent. As used herein, a fibrous substrate is durably water repellent if it demonstrates a spray rating of at least 50 after 10 launderings, when treated with 1% solids, preferably 0.6% solids, on fibrous substrate, using a treating composition of the present disclosure, as determined by the Spray Rating Test with launderings (and optional ironing), as described in the Examples Section. In certain embodiments, the spray rating is at least 70 after 10 launderings, or at least 80 after 10 launderings, or at least 80 after 20 launderings, when treated with 1% solids, preferably 0.6% solids, on fibrous substrate, using a treating composition of the present disclosure, as determined by the Spray Rating Test with launderings (and optional ironing), as described in the Examples Section.

Typically, an amount of treating composition is used to obtain a desired initial spray rating level and/or a desired spray rating level after laundering multiple times. In certain embodiments, the amount of treating composition is at least 0.2 wt-%, or at least 0.3 wt-%, or at least 0.4 wt-%, or at least 0.5 wt-%, or at least 0.6 wt-%, SOF (solids on fibrous substrate, e.g., fabric). In certain embodiments, the amount of treating composition is up to 2 wt-%, or up to 1.5 wt-%, or up to 1 wt-% SOF (solids on fibrous substrate, e.g., fabric).

Exemplary fibrous substrates include textile, leather, carpet, paper, and fabrics (nonwoven, woven, or knitted).

Compositions (e.g., treating compositions) of the present disclosure may be in the form of a concentrate, which may include up to 85 weight percent (wt-%) water, based on the total weight of the concentrated composition. Compositions (e.g., treating compositions) of the present disclosure may be in the form of a concentrate, which may include at least 65 wt-% water, based on the total weight of the concentrated composition.

Alternatively, concentrated compositions of the present disclosure may be diluted to any desired level to form a ready-to-use formulation.

In certain embodiments, compositions of the present disclosure include various combinations of Component (A), Component (B), and Component (C). That is, in certain embodiments, compositions include Component (A) and Component (B). In certain embodiments, compositions include Component (A) and Component (C). In certain embodiments, compositions include Component (B) and Component (C). In certain embodiments, compositions include Component (A), Component (B), and Component (C).

### Component (A) Polymeric Compounds and Preparation Thereof

In certain embodiments, Component (A) includes one or more polymeric compounds derived from the polymerization of at least one monomer comprising at least one hydrocarbon group having from 4 to 60 carbon atoms, 16 to 60 carbon atoms, 16 to 50 carbon atoms, 16 to 40 carbon atoms, or 16 to 30 carbon atoms.

In certain embodiments, Component (A) includes one or more polymeric compounds derived from the polymerization of at least one monomer of Formula (I), Formula (II), Formula (III), or Formula (IV), as described herein below.

Such polymeric compounds may be homopolymers or copolymers (including terpolymers, tetrapolymers, and the like).

In certain embodiments, Component (A) includes one or more polymeric compounds derived from the polymerization of at least one monomer of Formula (I):

R¹-D¹-C(O)C(R²)═CH₂ Formula (I).

In certain embodiments, the monomer of Formula (I) may be a (meth)acrylate monomer or a (meth)acrylamide monomer.

In Formula (I), R¹ represents a hydrocarbon group having 4 to 60 carbon atoms. In certain embodiments of Formula (I), R¹ represents a hydrocarbon group having 16 to 60 carbon atoms, or 16 to 30 carbon atoms.

In Formula (I), R² represents H or CH₃.

In Formula (I), D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)NHC(O)-L¹⁰-O—;
—C(O)—NH—;
—C(O)NHC(O)NH-L⁹-O—;
—NHC(O)NH—; and
—NH—; with the proviso that when D¹ is —NH—, the one or more polymeric compounds are derived from greater than 30 wt-% of monomers of Formula (I), based on the total weight of monomers.

In D¹ of Formula (I), each L¹, L², L³, L⁴, L⁵, L⁶, L⁷, L⁸, L⁹, and L¹⁰ is independently a branched or straight chain alkylene group having 2 to 10 carbon atoms. In certain embodiments of D¹ of Formula (I), each L¹, L², L³, L⁴, L⁵, L⁶, L⁷, L⁸, L⁹, and L¹⁰ is independently a straight chain (i.e., linear) alkylene group having 2 to 10 carbon atoms. In certain embodiments of D¹ of Formula (I) each L¹, L², L³, L⁴, L⁵, L⁶, L⁷, L⁸, L⁹, and L¹⁰ is independently an ethylene group, a butylene group, or a propylene group.

In certain embodiments of Formula (I), D¹ is —NH—, and the one or more polymeric compounds comprise a homopolymer of Formula (I).

In certain embodiments, Component (A) includes one or more polymeric compounds derived from the polymerization of at least one monomer of Formula (II):

R³-D²-C(O)C(R⁴)═CH₂ Formula (II).

In certain embodiments, the at least one monomer of Formula (II) is present in the one or more polymeric compounds in an amount of less than 30 wt-%, based on the total amount of monomers.

In Formula (II), R³ represents a hydrocarbon group having 4 to 60 carbon atoms.

In certain embodiments of Formula (II), R³ represents a hydrocarbon group having 16 to 60 carbon atoms, or 16 to 30 carbon atoms.

In Formula (II), R⁴ represents H or CH₃.

In Formula (II), D² is selected from:
—NH—C(O)OL¹¹-O—;
—O—C(O)NH-L¹²-O—;
—NHC(O)NH-L¹³-O—; and
—O—.

In D² of Formula (II), each L¹¹, L¹², and L¹³ is independently a branched or straight chain alkylene group having 2 to 10 carbon atoms. In certain embodiments of D² of Formula (II), each L¹¹, L¹², and L¹³ is independently a straight chain (i.e., linear) alkylene group having 2 to 10 carbon atoms.

In certain embodiments, Component (A) includes one or more polymeric compounds derived from the polymerization of at least one monomer of Formula (II):

R⁵—X¹(C(O)NH-L¹⁴-OC(O)C(R⁶)═CH₂ Formula (III).

In certain embodiments, Component (A) includes one or more polymeric compounds derived from the polymerization of at least one monomer of Formula (IV):

R⁷—X²—C(O)NH-Q¹-NH—C(O)O-L¹⁵-OC(O)C(R⁸)═CH₂ Formula (IV).

In Formulas (III) and (IV), R⁵ and R⁷ are independently a hydrocarbon group having from 4 to 60 carbon atoms. In certain embodiments of Formulas (III) and (IV), R⁵ and R⁷ are independently a linear or branched hydrocarbon group, and in certain embodiments, a linear hydrocarbon group, having from 4 to 60 carbon atoms, 16 to 60 carbon atoms, 16 to 50 carbon atoms, 16 to 40 carbon atoms, or 16 to 30 carbon atoms.

In Formulas (III) and (IV), R⁶ and R⁸ are independently H or CH₃.

In Formulas (II) and (IV). L¹⁴ and L¹⁵ are independently a branched or straight chain alkylene group having 2 to 10 carbon atoms, an arylene group (in some embodiments, an arylene group having 5 to 12 carbon atoms), or a combination thereof.

In Formulas (III) and (IV), X¹ is S or —N(R⁹) and X² is O, S, —NH, or —N(R⁹), wherein R⁹ is a hydrocarbon group (in certain embodiments, a linear or branched hydrocarbon group, and in certain embodiments, a linear hydrocarbon group) having 1 to 20 carbon atoms.

In Formula (IV), Q¹ is a divalent isocyanate residue.

In certain embodiments of Component (A), the one or more polymeric compounds have an average of greater than 20 repeating units (or at least 30 repeating units, or at least 50 repeating units, or at least 100 repeating units, or at least 200 repeating units, or at least 300 repeating units, or at least 400 repeating units, or at least 500 repeating units, or at least 600 repeating units, or at least 700 repeating units, or at least 800 repeating units, or at least 900 repeating units, or at least 1000 repeating units) of at least one monomer of Formula (I), Formula (II), Formula (III), or Formula (IV). In certain embodiments of Component (A), the one or more polymeric compounds have an average of greater than 20 repeating units (or at least 30 repeating units, or at least 50 repeating units, or at least 100 repeating units, or at least 200 repeating units, or at least 300 repeating units, or at least 400 repeating units, or at least 500 repeating units, or at least 600 repeating units, or at least 700 repeating units, or at least 800 repeating units, or at least 900 repeating units, or at least 1000 repeating units) of at least one monomer of Formula (I).

In certain embodiments, such polymeric compounds include up to 10,000 repeating units.

In certain embodiments of Component (A), the one or more polymeric compounds are derived from the polymerization of at least one monomer of Formula (I) and at least one monomer of Formula (I).

In certain embodiments of Component (A), the one or more polymeric compounds are derived from at least one monomer of the following formulas:

C₁₈H₃₇—NH—C(O)C(R²)═CH₂;

C₁₈H₃₇—NH—C(O)OCH₂CH₂OC(O)C(R²)═CH₂;

C₁₈H₃₇—NH—C(O)—NH—C(O)—C(R²)═CH₂; and

C₁₇H₃₅—C(O)OCH₂CH₂OC(O)C(R²)═CH₂.

wherein R² represents H or CH₃.

In certain embodiments of Component (A), the one or more polymeric compounds are derived from at least one monomer of the following formulas:

C₁₈H₃₇—NH—C(O)C(R²)═CH₂;

C₁₈H₃₇—NH—C(O)—NH—C(O)—C(R²)═CH₂; and

C₁₇H₃₅—C(O)OCH₂CH₂OC(O)C(R²)═CH₂.

wherein R² represents H or CH₃.

In certain embodiments of Component (A), the one or more polymeric compounds are derived from at least one monomer of the following formula:

C₁₈H₃₇—NH—C(O)C(R²)═CH₂

wherein R² represents H or CH₃. In certain embodiments of Component (A), the one or more polymeric compounds include a homopolymer of C₁₈H₃₇—NHC(O)C(H)═CH₂.

In certain embodiments of Component (A), the one or more polymeric compounds additionally include units derived from a monomer with one or more functional groups capable of undergoing additional reactions. In certain embodiments, the functional groups capable of undergoing additional reactions are selected from a polymerizable olefin group, an olefin group that can undergo a hydrosilation reaction, an epoxy group, a hydroxyl group, a halo group, a haloformyl group, an aziridino group, an acid group, a salt of an acid group, an amino group, a salt of an amino group, a quaternary ammonium group, a salt of a quaternary ammonium group, a blocked isocyanate group, a hydroxyalkyl group, a chlorinated hydroxyalkyl group, an N-methylol group, an acetoacetoxyalkyl group, and a combination thereof. Examples of such monomers include 2-hydroxyethyl(meth)acrylate, allyl(meth)acrylate, N-methylolacrylamide, glycidylmethacrylate, 3-chloro 2-hydroxypropylacrylate or the reaction product of 2-isocyanatoethyl(meth)acrylate with methylethylketoxime (MEKO).

In certain embodiments of Component (A), the one or more polymeric compounds have a weight average molecular weight of greater than 20,000 Daltons. In certain embodiments of Component (A), the one or more polymeric compounds have a weight average molecular weight of up to 500,000 Daltons.

Techniques and conditions for making the monomers of Formulas (I), (II), (III), and (IV) described herein would be well known to one of skill in the art. For example, a suitable monomer reactant (e.g., (meth)acrylic acid, (meth)acrylamide, hydroxyalkyl acrylate) and an isocyanate reactant (e.g., stearyl isocyanate) can be combined with or without an appropriate catalyst.

A catalyst in an appropriate amount (e.g., 500 ppm) may be used, but is not mandatory (particularly if higher temperatures are used). Exemplary catalysts include dibutyl tin dilaurate (DBTDL) and bismuth neodecanoate (e.g., Shepherd Bicat 8108M, ABCR Bismuth (III) neodecanoate, superconductor grade, about 60% in neodecanoic acid (15-20% Bi), or Strem Chemicals Bismuth (III) neodecanoate, superconductor grade, about 60% in neodecanoic acid (15-20% Bi)).

The reaction to form a (meth)acrylate containing an isocyanate-derived group (as used below in Components (B) and (C)), for example, can typically be carried out in a temperature range of 40° C. to 100° C., or 70° C. to 100° C., or 75° C. to 95° C. preferably under dry conditions (e.g., dry air). If no catalyst is used, a reaction temperature of 70° C. to 100° C. is preferred. Typically, the reaction is carried out in 1-24 hours, or 4-15 hours.

To prevent unwanted radical polymerization during the synthesis of the monomers, a stabilizer in an appropriate amount (e.g., 50 to 500 ppm), such as 3,5-di-tert-butyl-4-hydroxy-toluene (BHT), 4-methoxyphenol (MOP), or hydroquinone (HQ) may be used, but is not mandatory.

Further examples for making suitable monomers are elaborated in the Examples Section.

In certain embodiments, the polymeric compounds of Component (A) can be prepared by a free-radical polymerization.

In order to prepare the polymeric compounds, a free-radical initiator may be used to initiate the polymerization. Free-radical initiators include those known in the art and include, in particular, azo compounds such as 2,2′-azobis(2-methylpropionamidine) dihydrochloride, 2,2′-azobis(2-methylbutyronitrile), 2,2′-azobisisobutyronitrile (AIBN) and 2,2′-azobis(2-cyanopentane), and the like, hydroperoxides such as cumene, t-butyl- and t-amyl-hydroperoxide, and the like, peroxyesters such as t-butylperbenzoate, di-t-butylperoxyphtalate, and the like, and diacylperoxides such as benzoyl peroxide, lauroyl peroxide, and the like.

The polymerization may be carried out in a wide variety of solvents suitable for organic free-radical reactions. Examples of suitable solvents include aliphatic and alicyclic hydrocarbons (e.g., hexane, heptane, cyclohexane), ethers (e.g., diethylether, glyme, diglyme, diisopropyl ether), esters (e.g., ethylacetate, butylacetate), ketones (e.g., acetone, methylethyl ketone, methyl isobutyl ketone), and mixtures thereof.

The polymerizations can also be carried out in aqueous media, such as in an emulsion polymerization or mini-emulsion polymerization, using the appropriate emulsifiers and initiators known to those skilled in the art. Emulsifiers include nonionic, cationic, amphoteric, or anionic surfactants, such as sodium alkylsulphonates, sodium alkylbenzenesulphonates, sodium dialkylsulphosuccinates, (C12-C18)alkylalcohol-ethyleneoxide adducts, polyethoxylatednonylphenois, or alkylquaternary ammoniumethoxylates.

The polymerization reaction can be carried out at a temperature suitable for conducting a free-radical polymerization reaction. Particular temperatures and solvents for use can be easily selected by those skilled in the art based on considerations such as the solubility of reagents, the temperature required for the use of a particular initiator, molecular weight desired, and the like. While it is not practical to enumerate a particular temperature suitable for all initiators and all solvents, generally suitable temperatures are 30° C. to 150° C. In certain embodiments, the temperature is 55° C. to 95° C., or 75° C. to 85° C. Reaction times typically are within 1 to 24 hours, and often within 4 to 15 hours.

### Component (B) Compounds and Preparation Thereof

In certain embodiments, Component (B) includes one or more compounds derived from reaction, in one or more steps, of components including:
(i) at least one isocyanate-reactive (i.e., functionalized) oligomer comprising 2 to 20 repeating units, or an alcohol, amine, acid, amide, or thiol, having at least one linear or branched hydrocarbon group having from 4 to 60 carbon atoms and optionally one or more ester groups (in certain embodiments, at least one isocyanate-reactive oligomer comprising 2 to 20 repeating units, or an amine, acid, or amide, having at least one linear or branched hydrocarbon group having from 4 to 60 carbon atoms and optionally one or more ester groups, and in certain embodiments, at least one isocyanate-reactive oligomer comprising 2 to 20 repeating units);
(ii) at least one polyisocyanate;
(iii) optionally at least one additional mono-, di-, or poly-functional isocyanate-reactive compound; and
(iv) optionally at least one isocyanate blocking agent.

Typically, one or more compounds of Component (B) used in compositions of the present disclosure may be prepared in a one-step reaction, a two-step reaction, or optionally three-step reaction, although it will generally not be required to separate reaction products after the individual steps. That is, the reaction may be carried out in three steps in a single reactor.

In certain embodiments, an alcohol having at least one linear or branched hydrocarbon group having from 16 to 40 carbon atoms and optionally one or more ester groups (e.g., ISOFOL 28, ISOFOL 32, ISOFOL 36, UNILIN 350, sorbitan tristearate, and the reaction product of 1 mole of citric acid and 3 moles of stearyl alcohol) is reacted with an equivalent amount of a polyisocyanate.

In certain embodiments, in a first step, a functionalized oligomer having from 2 to 20 repeating units is prepared. In a second step, this functionalized oligomer is reacted with a polyisocyanate, optionally in the presence of an additional isocyanate-reactive compound. Thus, the term “functionalized oligomer” (also referred to as isocyanate-reactive oligomer) means an oligomer that contains a functional group capable of reacting with an isocyanate. Examples of such groups include alcohol, amine, thiol, acid, or amide groups.

In certain embodiments, in a first step, a functionalized oligomer having from 2 to 20 repeating units is prepared. In a second step, this functionalized oligomer is reacted with an excess isocyanate (typically, a poly-isocyanate) to form an isocyanate-containing oligomer (i.e., an oligomer having at least one isocyanate end group). In a third step, the isocyanate-containing oligomer (i.e., oligomer with isocyanate end groups) is further reacted with an isocyanate blocking agent. Thus, the reaction product of the second step, i.e., the oligomer that includes at least one isocyanate group (e.g., an isocyanate end group), may be formed in the reaction mixture without being isolated (i.e., it is formed in situ).

In certain embodiments of Component (B), less than 40% of the isocyanate groups are reacted with acid and/or amide groups.

In certain embodiments, the isocyanate-reactive oligomer is made by the radical-initiated reaction of at least one (meth)acrylate or (meth)acrylamide monomer in the presence of at least one mercaptan, which may or may not be functionalized. In certain embodiments, the mercaptan is functionalized with at least one isocyanate-reactive group (e.g., alcohol, amine, acid, or amide group). In certain embodiments, the molar ratio of the mercaptan to the (meth)acrylate or (meth)acrylamide monomer is 1:4 to 1:20, or 1:8 to 1:16.

Examples of mercaptans include mono- or di-functional hydroxyl-, amino-, acid-, or amide-functionalized mercaptans (as chain transfer agents), in order to prepare mono- or di-functionalized oligomers, respectively. Examples of monofunctional mercaptans (as chain transfer agents) include 2-mercaptoethanol, 3-mercapto-2-butanol, 3-mercapto-2-propanol, 3-mercapto-1-propanol, 2-mercapto-ethylamine, 3-mercaptopropionic acid, and 16-mercaptohexadecanoic acid. Particularly suitable monofunctional chain transfer agents include 2-mercaptoethanol, 3-mercaptopropionic acid, and 16-mercaptohexadecanoic acid. Examples of difunctional mercaptans (as chain transfer agents) include those having two hydroxyl or amino groups or one hydroxyl and one amino group. A suitable example of a difunctional chain transfer agent is 3-mercapto-1,2-propanediol (thioglycerol).

In certain embodiments, non-functional mercaptans are used, i.e., mercaptans that do not contain an additional isocyanate-reactive group. In order to make a functionalized oligomer with such mercaptans, the non-functional mercaptan is reacted with a mixture of (meth)acrylates and/or (meth)acrylamides, wherein at least one (meth)acrylate and/or (meth)acrylamide has a functional group capable of reacting with an isocyanate group. Examples of non-functional mercaptans include octylmercaptan, dodecylmercaptan, octadecylmercaptopropionate, and octadecylmercaptan. Examples of functionalized (meth)acrylates capable of undergoing a reaction with an isocyanate group, include for example, are 2-hydroxyethyl (meth)acrylate, 3-hydroxypropyl (Meth)acrylate, 4-hydroxybutyl (meth)acrylate, and 2-carboxyethylacrylate.

In certain embodiments, the at least one (meth)acrylate or (meth)acrylamide monomer for making the isocyanate-reactive oligomer includes at least one hydrocarbon group (in certain embodiments, a linear or branched hydrocarbon group, and in certain embodiments, a linear hydrocarbon group) having from 4 to 60 carbon atoms, 16 to 60 carbon atoms, 16 to 50 carbon atoms, 16 to 40 carbon atoms, or 16 to 30 carbon atoms. In certain embodiments, the at least one (meth)acrylate or (meth)acrylamide monomer includes at least one hydrocarbon group (in certain embodiments, a linear or branched hydrocarbon group, and in certain embodiments, a linear hydrocarbon group) having from 4 to 60 carbon atoms, 16 to 60 carbon atoms, 16 to 50 carbon atoms, 16 to 40 carbon atoms, or 16 to 30 carbon atoms, and at least one isocyanate-derived group.

In this context, an “isocyanate-derived group” is a chemical linking group obtained by reaction of an isocyanate-containing compound with a compound containing a functional group capable of undergoing a reaction with an isocyanate, with or without the use of a catalyst. Examples of such groups include, for example, a urethane group (obtained by reaction of an isocyanate-containing compound with an alcohol-containing compound), a urea group (obtained by reaction of an isocyanate-containing compound with an amine-containing compound), an amide group (obtained by reaction of an isocyanate-containing compound with a carboxylic acid-containing compound), or an acylurea group (obtained by reaction of an isocyanate-containing compound with an amide-containing compound).

In certain embodiments, the monomer for making the isocyanate-reactive oligomers used to make the one or more compounds of Component (B) is at least one of Formulas (T), (II), (II), or (IV) (described above for Component (A)). In certain embodiments, if a monomer of Formula (II) is used in making the isocyanate reactive oligomer, it is used in an amount of less than 30 wt-%, based on the total amount of monomers.

In order to prepare the functionalized oligomers (i.e., isocyanate-reactive oligomers), a free-radical initiator may be used to initiate the oligomerization. Free-radical initiators include those known in the art and include, in particular, azo compounds such as 2,2′-azobis(2-methylbutyronitrile), 2,2′-azobisisobutyronitrile (AIBN) and 2,2′-azobis(2-cyanopentane), and the like, hydroperoxides such as cumene, t-butyl- and t-amyl-hydroperoxide, and the like, peroxyesters such as t-butylperbenzoate, di-t-butylperoxyphtalate, and the like, and diacylperoxides such as benzoyl peroxide, lauroyl peroxide, and the like.

The oligomerization reaction to form a functionalized (i.e., isocyanate-reactive) oligomer may be carried out in a wide variety of solvents suitable for organic free-radical reactions. Particularly suitable solvents are solvents that do not interfere with the isocyanate reactions in the subsequent step(s). The reactants can be present in the solvent at any suitable concentration, e.g., from about 5 percent to about 90 percent by weight, based on the total weight of the reaction mixture. Examples of suitable solvents include aliphatic and alicyclic hydrocarbons (e.g., hexane, heptane, cyclohexane), ethers (e.g. diethylether, glyme, diglyme, diisopropyl ether), aromatic hydrocarbons (e.g., toluene), esters (e.g., ethylacetate, butylacetate), ketones (e.g., acetone, methylethyl ketone, methyl isobutyl ketone), and mixtures thereof.

The oligomerization reaction to form a functionalized (i.e., isocyanate-reactive) oligomer can be carried out at a temperature suitable for conducting a free-radical oligomerization reaction. Particular temperatures and solvents for use can be easily selected by those skilled in the art based on considerations such as the solubility of reagents, the temperature required for the use of a particular initiator, molecular weight desired, and the like. While it is not practical to enumerate a particular temperature suitable for all initiators and all solvents, generally suitable temperatures between 30° C. and 150° C. In certain embodiments, the temperature is between 55° C. and 90° C., or between 75° C. and 85° C. Reaction times typically are within 1 to 24 hours, and often within 3 to 15 hours.

In certain embodiments, the isocyanate-reactive oligomer is of Formula (V):

Y¹—[CH₂—C(R²)C(O)-D¹-R¹]ₘS—R¹⁰—(T¹)ₚ Formula (V).

In Formula (V), Y¹ is H or an initiator residue.

In Formula (V), R¹ is a hydrocarbon group having 4 to 60 carbon atoms, 16 to 60 carbon atoms, 16 to 50 carbon atoms, 16 to 40 carbon atoms, or 16 to 30 carbon atoms.

In Formula (V), R² is independently H or CH₃.

In Formula (V), D¹ is selected from:
—C(O)O-L¹-O—;
—OC(O)-L²-O—;
—OC(O)-L³C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—N—; with the proviso that when D¹ is —N—, the isocyanate-reactive oligomer comprises greater than 30 wt-% of monomeric units that include —NH—, based on the total weight of monomeric units.

In certain embodiments, D¹ is —NH—, and the oligomer includes 100% of monomeric units that include —NH—.

In D¹ of Formula (V), each L¹, L², L³, L⁴, L⁵, L⁶, L⁷, L⁸, L⁹, and L¹⁰ is independently a branched or straight chain alkylene group having 2 to 10 carbon atoms. In certain embodiments, each L¹, L², L³, L⁴, L⁵, L⁶, L⁷, L⁸, L⁹, and L¹⁰ is independently a straight chain (i.e., linear) alkylene group having 2 to 10 carbon atoms.

In Formula (V), S is sulfur.

In Formula (V), R¹⁰ is a divalent or trivalent linking group having 1 to 10 carbon atoms.

In Formula (V), T is —C(O)OH, —C(O)NH₂, —OH, —NH₂, or —NH(R¹¹), wherein R¹¹ is H or a hydrocarbon group having 1 to 10 carbon atoms;

In Formula (V), m is an integer of 2 to 20.

In Formula (V), p is independently 1 or 2.

In certain embodiments, the isocyanate-reactive oligomer is of Formula (VI):

Y²—[CH₂—C(R⁴)C(O)-D²-R³]ₘ—S—R¹²-(T²)ₚ Formula (VI).

In Formula (VI), Y² is H or an initiator residue.

In Formula (VI), R³ is a hydrocarbon group having 4 to 60 carbon atoms, 16 to 60 carbon atoms, 16 to 50 carbon atoms, 16 to 40 carbon atoms, or 16 to 30 carbon atoms.

In Formula (VI), R⁴ is independently H or CH₃.

In Formula (VI), D² is selected from:
—NHC(O)OL¹¹-O—;
—O—C(O)NH-L¹²-O—,
—NHC(O)NH-L¹³-O—; and
—O—.

In Formula (VI), each L¹¹, L¹², and L¹³ is independently a branched or straight chain alkylene group having 2 to 10 carbon atoms. In certain embodiments, each L¹¹, L¹², and L¹³ is independently a straight chain (i.e., linear) alkylene group having 2 to 10 carbon atoms.

In Formula (VI), S is sulfur.

In Formula (VI), R¹² is a divalent or trivalent linking group having 1 to 10 carbon atoms.

In Formula (VI), T² is —C(O)OH, —C(O)NH₂, —OH, NH or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality.

In Formula (VI), m is an integer of 2 to 20.

In Formula (VI), p is independently 1 or 2.

In certain embodiments, the weight average molecular weight of the functionalized (i.e., isocyanate-reactive) oligomers is at least 600, or at least 1500, or at least 2000 Daltons. In certain embodiments, the weight average molecular weight of the functionalized oligomers is up to 20,000, or up to 10,000, or up to 5,000 Daltons.

In a second step, an isocyanate-containing oligomer (i.e., an isocyanate oligomer having at least one isocyanate end group) is prepared by a condensation reaction of the functionalized (i.e., isocyanate-reactive) oligomer with a polyisocyanate. The reaction product of such condensation reaction is typically a mixture of isocyanate-containing oligomers.

In certain embodiments, in the components for making the one or more compounds of Component (B), the at least one polyisocyanate includes aromatic and/or aliphatic polyisocyanates, such as an aromatic or aliphatic diisocyanate, an aromatic or aliphatic triisocyanate, an aromatic or aliphatic polymeric isocyanate, or a mixture thereof. Examples of such polyisocyanates include 2,4-toluene diisocyanate, 2,6-toluene diisocyanate, o, m, and p-xylylene diisocyanate, 1,6-hexamethylene diisocyanate, 1,4-isophorone diisocyanate, 4,4′-diisocyanatodiphenylether, 3,3′-dichloro-4,4′-diisocyanatodiphenylmethane, 4,4′-diphenyldiisocyanate, 4,4′-diisocyanatodibenzyl, 3,3-dimethoxy-4,4′-diisocyanatodiphenyl, 3,3′-dimethyl-4,4′-diisocyanatodiphenyl, 2,2′-dichloro-5,5′-dimethoxy-4,4′-diisocyanato diphenyl, 1,3-diisocyanatobenzene, 1,2-naphthylene diisocyanate, 4-chloro-1,2-naphthylene diisocyanate, 1,3-naphthylene diisocyanate, 1,8-dinitro-2,7-naphthylene diisocyanate, tri-(4-isocyanatophenyl)-methane, polymethylenepolyphenylisocyanate (PAPI), and their respective biurets or isocyanurates.

In certain embodiments, the components for making the one or more compounds of Component (B) include (iii) at least one additional mono-, di-, or poly-functional isocyanate-reactive compound.

In certain embodiments, the additional mono-, di-, or poly-functional isocyanate-reactive compound includes a compound including: a hydrocarbon group having 4 to 60 carbon atoms (or 16 to 60 carbon atoms, or 16 to 50 carbon atoms, or 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms; or a combination thereof.

In certain embodiments, the additional mono-, di-, or poly-functional isocyanate-reactive compound includes a mono- di-, or poly-functional alcohol, thiol, amine, acid, or amide.

Examples of alcohol-containing compounds include methanol, ethanol, n-propylalcohol, isopropyl alcohol, n-butyl alcohol, isobutyl alcohol, t-butyl alcohol, n-amyl alcohol, t-amyl alcohol, 2-ethylhexanol, glycidol, stearyl alcohol, (iso)stearyl alcohol, behenyl alcohol, branched long chain alkanols, such as ISOFOL alcohols (2-alkyl alkanols having C-14 to C-32 alkyl chains, available from Sasol, Germany), alcohols comprising poly(oyalkylene) groups, such as methyl or ethyl ether of polyethyleneglycol, hydroxyl-terminated methyl or ethyl ether of random or block copolymer of ethyleneoxide and/or propyleneoxide and polysiloxane (e.g., polydimethylsiloxane) group-containing alcohols. Further examples include diols, triols, and polyols such as 1,4-butanediol, 1,6-hexanediol, 1-10-decanediol, 4,4′-isopropylidene diphenol (Bisphenol A), glycerol, pentaerythritol, dipentaerythritol; polyester diols, such as polycaprolactone diol, fatty acid dimer diols and poly(oxy)alkylenediols with an oxyalkylene group having 2 to 4 carbon atoms, such as —OCH₂CH₂—, —O(CH₂)₄—, —OCH₂CH₂CH₂—, —OCH(CH₃)CH₂—, and —OCH(CH₃)CH(CH₃)— (the oxyalkylene units in said poly(oxyalkylene) may be the same, as in polypropyleneglycol, or present as a mixture), and ester diols, such as glycerol monostearate and polysiloxane-containing (e.g., polydimethylsiloxane-containing) diols.

Examples of thiol-containing compounds include octadecylmercaptan, dodecylmercaptan, octadecylmercaptopropionate, 1,4-butanedithiol, and 1,6-hexanedithiol.

Examples of amine-containing compounds include octadecylamine, di(octadecyl)amine, 1,6-hexamethylenediamine, amino-terminated polyethyleneoxide or propyleneoxide or copolymers thereof, amino-terminated methyl or ethylethers of polyethyleneoxide or polypropyleneoxide or copolymers thereof and amino group-terminated polysiloxanes, for example, polydimethylsiloxanes.

Examples of acid-containing compounds include octadecyl (stearic) acid, docosanoic (or behenic acid), adipic acid, dodecanedioic acid, and octadecanedioic acid.

Examples of amide-containing compounds include octadecyl (stearyl) amide, isostearyl amide, and octane amide.

The condensation reaction of the functionalized (i.e., isocyanate-reactive) oligomer with a polyisocyanate (in one embodiment an excess polyisocyanate) may be carried out under conventional conditions well-known to those skilled in the art. In certain embodiments, the condensation reaction is carried out under dry conditions in a polar solvent such as ethylacetate, acetone, methyl isobutyl ketone, and the like. Suitable reaction temperatures will be easily determined by those skilled in the art based on the particular reagents, solvents, and catalysts being used. While it is not practical to enumerate particular temperatures suitable for all situations, generally suitable temperatures are room temperature to 120° C.

In certain embodiments the condensation reaction is carried out without catalyst present. In certain embodiments the condensation reaction is carried out with catalyst present; these catalysts are well known to those skilled in the art. Depending on the isocyanate-reactive group, a suitable catalyst can be selected from, for example, tin-catalysts such as dibutyltindilaurate or tinoctoate, zirconium-catalysts such as zirconium isopropoxide; or magnesium halides, such as magnesium chloride.

The condensation reaction typically results in a mixture of compounds. These compounds may be used directly in a treating composition of the present disclosure.

In certain embodiments, the components for making the one or more compounds of Component (B) include (iv) a blocked isocyanate group. Such blocked isocyanate groups are the result of a reaction of an isocyanate-containing oligomer (obtained from a condensation reaction of the isocyanate reactive oligomer in the presence of excess polyisocyanate) with a blocking agent.

Such blocked isocyanates are particularly preferred because they impart a durable property when such blocked compound is applied to a fibrous substrate. This is generally because the blocking agent is removable from the isocyanate under the thermal conditions employed upon curing a fibrous substrate treated with a compound containing the blocked isocyanato group.

Conventional isocyanate blocking agents include arylalcohols (e.g., phenol, cresols, nitrophenols, o- and p-chlorophenol, naphthols, 4-hydroxybiphenyl); C2 to CS alkanone oximes (e.g., acetoneoxime, butanoneoxime); arylthiols (e.g., thiophenol); organic active hydrogen compounds (e.g., diethyl malonate, acetylacetone, ethyl acetoacetate, ethyl cyanoacetate, epsilon-caprolactam); sodium bisulfite; and hydroxylamine.

Particularly preferred blocked isocyanates include those blocked with oxime blocking agents such as C2 to CS alkanone oximes, particularly 2-butanoneoxime. That is, preferred blocked isocyanate groups are oxime-derived groups. Such blocked isocyanates can be de-blocked at a relatively low temperature, for example, during the process of curing a fibrous substrate that has been treated with the compound comprising the blocked isocyanate group.

Various combinations of blocking agents can be used if desired to make compounds of Component (B) of the disclosure. In certain embodiments, if blocking agents are used, up to 70%, or up to 60%, of isocyanate equivalents are blocked by one or more blocking groups. In certain embodiments, if blocking agents are used, at least 10%, or at least 20%, or at least 30%, or at least 40%, of isocyanate equivalents are blocked by one or more blocking groups.

This step of blocking the isocyanate-containing oligomer under conventional conditions is well-known to those skilled in the art. In certain embodiments, the blocking reaction is carried out under dry conditions in a polar solvent such as ethylacetate, acetone, methyl isobutyl ketone, and the like. Suitable reaction temperatures will be easily determined by those skilled in the art based on the particular reagents, solvents, and catalysts being used. While it is not practical to enumerate particular temperatures suitable for all situations, generally suitable temperatures are room temperature to 120° C.

### Component (C) Polycarbodiimides and Preparation Thereof

Component (C) includes one or more polycarbodiimide compounds (i.e., a compound that includes one or more groups of the formula (—N═C═N—)).

In certain embodiments, the one or more polycarbodiimide compounds of Component (C) are derived from a carbodiimidization reaction, in one or more steps, of components including:
(i) at least one isocyanate-reactive oligomer (i.e., functionalized oligomer) comprising 2 to 20 repeating units, or an alcohol, amine, acid, amide, or thiol, having at least one linear or branched hydrocarbon group having from 4 to 60 carbon atoms (or 16 to 60 carbon atoms, or 16 to 50 carbon atoms, or 16 to 40 carbon atoms, or 16 to 30 carbon atoms) and optionally one or more ester groups (in certain embodiments, at least one isocyanate-reactive oligomer comprising 2 to 20 repeating units, or an amine, acid, or amide, having at least one linear or branched hydrocarbon group having from 4 to 60 carbon atoms and optionally one or more ester groups, and in certain embodiments, at least one isocyanate-reactive oligomer comprising 2 to 20 repeating units);
(ii) at least one polyisocyanate; and
(iii) optionally at least one additional mono-, di-, or poly-functional isocyanate-reactive compound.

Typically, a polycarbodiimide compound of the present disclosure, or mixtures thereof, may be prepared in a three-step reaction, although it will generally not be required to separate reaction products after the individual steps. That is, the reaction may be carried out in three steps in a single reactor.

In certain embodiments, an alcohol having at least one linear or branched hydrocarbon group having from 16 to 40 carbon atoms and optionally one or more ester groups (e.g. ISOFOL 28, ISOFOL 32, ISOFOL 36, UNILIN 350, sorbitan tristearate, and the reaction product of 1 mole citric acid and 3 moles of stearyl alcohol) is reacted with an isocyanate to form an isocyanate-containing compound. In a second step, the isocyanate-containing compound is further reacted in a carbodiimidization reaction to form a carbodiimide.

In certain embodiments, in a first step, a functionalized oligomer having at least two repeating units is prepared. In a second step, this functionalized oligomer is reacted with a polyisocyanate to form an isocyanate-containing oligomer (i.e., an oligomer having at least one isocyanate end group and at least two repeating units) as described above for Component (B). In a third step, the isocyanate-containing oligomer (i.e., oligomer with isocyanate end groups) is further reacted in a carbodiimidization reaction to form a polycarbodiimide. Thus, the reaction product of the second step, i.e., the oligomer that includes at least one isocyanate end group and at least two repeating units, may be formed in the reaction mixture without being isolated (i.e., it is formed in situ).

The isocyanate-reactive oligomers (i.e., functionalized oligomers) used for making the one or more polycarbodiimide compounds of Component (C) can be made as described above for Component (B) using monomers as described above for Component (A). Also, the isocyanate-containing oligomers (i.e., oligomers with isocyanate end groups) that further undergo a carbodiimidization reaction can be made as described above for Component (B).

In certain embodiments, the isocyanate-reactive oligomer is made by the radical-initiated reaction of at least one (meth)acrylate or (meth)acrylamide monomer in the presence of at least one mercaptan, which may or may not be functionalized. In certain embodiments, the mercaptan is functionalized with at least one isocyanate-reactive group (e.g., alcohol or amine group). In certain embodiments, the molar ratio of the mercaptan to the (meth)acrylate or (meth)acrylamide monomer is 1:4 to 1:20, or 1:8 to 1:16.

Examples of mercaptans include those listed above in Component (B).

In certain embodiments, the at least one (meth)acrylate or (meth)acrylamide monomer for making the isocyanate-reactive oligomer includes at least one hydrocarbon group (in certain embodiments, a linear or branched hydrocarbon group, and in certain embodiments, a linear hydrocarbon group) having from 4 to 60 carbon atoms, 16 to 60 carbon atoms, 16 to 50 carbon atoms, 16 to 40 carbon atoms, or 16 to 30 carbon atoms. In certain embodiments, the at least one (meth)acrylate or (meth)acrylamide monomer includes at least one hydrocarbon group (in certain embodiments, a linear or branched hydrocarbon group, and in certain embodiments, a linear hydrocarbon group) having from 4 to 60 carbon atoms, 16 to 60 carbon atoms, 16 to 50 carbon atoms, 16 to 40 carbon atoms, or 16 to 30 carbon atoms, and at least one isocyanate-derived group.

In certain embodiments, the monomers for making the isocyanate-reactive oligomers used to make the one or more compounds of Component (C) are at least one of Formulas (I), (II), (III), or (IV) (described above for Component (B)). In certain embodiments, if a monomer of Formula (II) is used in making the isocyanate reactive oligomer, it is used in an amount of less than 30 wt-%, based on the total amount of monomers.

In order to prepare the functionalized oligomers (i.e., isocyanate-reactive oligomers), a free-radical initiator may be used to initiate the oligomerization as described above for Component (B).

In certain embodiments, the isocyanate-reactive oligomer used to make the polycarbodiimide compounds of Component (C) is of Formula (V) and/or Formula (VI) (as described above for Component (B)).

In a second step, an isocyanate-containing oligomer (i.e., an isocyanate oligomer having at least one isocyanate end group) is prepared by a condensation reaction of the functionalized (i.e., isocyanate-reactive) oligomer with an excess of a polyisocyanate as described above for Component (B). The reaction product of such condensation reaction is typically a mixture of isocyanate-containing oligomers.

In certain embodiments, the components for making the one or more compounds of Component (C) include (iii) at least one additional mono-, di-, or poly-functional isocyanate-reactive compound. In certain embodiments, the additional mono-, di-, or poly-functional isocyanate-reactive compound includes those described above for Component (B).

In a third step, polycarbodiimide compounds (Component (C)) used in the compositions of the present disclosure may be formed by a carbodiimidization reaction in the presence of a suitable catalyst.

Representative examples of suitable catalysts are described, for example, in U.S. Pat. Nos. 2,941,988, 3,862,989, and 3,896,251. Examples include 3-methyl-1-phenyl-2-phospholene-1-oxide (MPPO), 1-ethyl-3-phospholine, 1-ethyl-3-methyl-3-phospholine-1-oxide, I-ethyl-3-methyl-3-phospholine-1-sulfide, I-ethyl-3-methyl-phospholidine, I-ethyl-3-methyl-phospholidine-1-oxide, 3-methyl-1-phenyl-3-phospholine-1-oxide and bicyclic terpene alkyl or hydrocarbyl aryl phosphine oxide or camphene phenyl phosphine oxide.

The particular amount of catalyst used will depend to a large extent on the reactivity of the catalyst itself and the isocyanate. A concentration range of 0.05-5 parts of catalyst per 100 parts of oligomer having at least one isocyanate group is generally suitable.

This third step of carbodiimidization may be carried out under conventional conditions well-known to those skilled in the art. In certain embodiments, the carbodiimidication reaction is carried out under dry conditions in a polar solvent such as ethylacetate, acetone, methyl isobutyl ketone, and the like. Suitable reaction temperatures will be easily determined by those skilled in the art based on the particular reagents, solvents, and catalysts being used. While it is not practical to enumerate particular temperatures suitable for all situations, generally suitable temperatures are 70° C. to 100° C. In certain embodiments, the reaction is carried out at a temperature of 75° C. to 95° C.

In one embodiment of the three-step process, steps 2 and 3 are carried out at the same time. That is, the condensation reaction (step 2) and carbodiimide reaction (step 3) are done at the same time.

In certain embodiments, the one or more polycarbodiimide compounds of Component (C) have the following formula:

Q²-(X³—C(O)NH-(A¹-(N═C═N))ᵣ-A²-NHC(O)X⁴)ₛ-Q³ Formula (VII).

In Formula (VII), X³ is a chemical bond, —O—, —NH—, —N(R¹⁴), —S— or —C(O)NH—, and X⁴ is a chemical bond. —O—, —NH—, —N(R¹⁴), —S— or —NHC(O)—, wherein R¹⁴ is a hydrocarbon group (e.g., an alkyl group) having 1 to 20 carbon atoms (e.g., methyl, ethyl, octyl, and octadecyl).

In Formula (VII), A¹ and A² each represents the residue of an organic di-isocyanate compound.

In Formula (VII), s is 1 or 2.

In Formula (VII), r is 2 to 10.

In Formula (VII), Q² and Q³ are independently selected from: a hydrocarbon group (in certain embodiments, a linear or branched hydrocarbon group) having at least 2 carbon atoms (in certain embodiments, 2 to 60 carbon atoms); and a group having the formula —(CH₂)ₐ—S—U (Formula (VIII)).

In Formula (VIII), a is an integer from 1 to 10.

In Formula (VIII), S is sulfur.

In Formula (VIII), U is an oligomeric group comprising 2 to 20 repeating units of monomers selected from at least one of Formulas (I), (II), (III), and (IV) (as described above for Component (A)). In certain embodiments, if a monomer of Formula (II) is used in making the isocyanate reactive oligomer, it is used in an amount of less than 30 wt-%, based on the total amount of monomers.

In certain embodiments in Formula (VII), X³ and X⁴ are each —O—, s is 1, and r is 3.

### Treating Compositions

Compositions of the present disclosure are typically treating compositions. In certain embodiments, such treating composition is a fluorine-free treating composition. In certain embodiments, such treating composition is an aqueous dispersion.

In certain embodiments, a fluorine-free treating composition further includes one or more additives selected from a paraffin wax, a surfactant, a coalescing solvent, an anti-freeze solvent, an emulsifier, and a stabilizer against one or more microorganisms.

Components A, B, and/or C, may be dispersed in water using a surfactant or mixture of surfactants in an amount sufficient to stabilize the dispersion. If one or more of the components are made in solution in a solvent, it can be dispersed in water through vigorously mixing and homogenizing with the help of a surfactant or emulsifier and subsequent homogenization, for example, by a Manton Gaulin homogenizer or ultrasound homogenizer. An organic solvent-free dispersion can then be obtained by subsequent distillation of the solvent.

A typical dispersion will contain water in an amount of 100 to 1000 parts by weight based on 100 parts by weight of Components A, B, and/or C. In certain embodiments, the surfactant or mixture of surfactants is present in an amount of 1 to 25 parts by weight, or 5 to 15 parts by weight, based on 100 parts by weight of Components A, B, and/or C.

Treating compositions of the present disclosure can include conventional cationic, nonionic, anionic, and/or zwitterionic (i.e., amphoteric) surfactants (i.e., emulsifiers). A mixture of surfactants may be used, e.g., containing nonionic and ionic surfactants. Suitable nonionic surfactants can have high or low 1-LB values, such as TERGITOL's, TWEEN's, and the like. Suitable cationic surfactants include mono- or bi-tail ammonium salts, such as ETHOQUAD C-12 and ARMOCARE VGH 70. Suitable anionic surfactants include sulfonic and carboxylic aliphatic compounds and their salts, such as sodium dodecylbenzene sulphonate (available from Rhodia, France), and the like. Suitable amphoteric surfactants include cocobetaines, sulphobetaines, amine-oxides, and the like.

In certain embodiments, surfactants suitable for use in the treating compositions of the present disclosure are described in International Publication No. WO 2013/162704.

In certain embodiments of the present disclosure, a treating composition includes Component (A). In certain embodiments, a treating composition includes Component (A) and at least one of Component (B) and Component (C).

Certain embodiments of compositions (e.g., fluorine-free treating compositions) of the present disclosure include: from 1 wt-% to 99 wt-% of Component (A), based on the total weight of Component (A) and Components (B) and/or (C) and from 1 wt-% to 99 wt-% of Component (B), Component (C), or both, based on the total weight of Component (A) and Components (B) and/or (C).

Certain embodiments of compositions (e.g., fluorine-free treating compositions) of the present disclosure include: 10 wt-% to 90 wt-% of Component (A), based on the total weight of Component (A) and Components (B) and/or (C); and 10 wt-% to 90 wt-% of Component (B), Component (C), or both, based on the total weight of Component (A) and Components (B) and/or (C).

Certain embodiments of compositions (e.g. fluorine-free treating compositions) of the present disclosure include: 20 wt-% to 80 wt-% Component (A), based on the total weight of Component (A) and Components (B) and/or (C); and 20 wt-% to 80 wt-% Component (B), Component (C), or both, based on the total weight of Component (A) and Components (B) and/or (C).

Certain embodiments of compositions (e.g., fluorine-free treating compositions) of the present disclosure include: 30 wt-% to 70 wt-% Component (A), based on the total weight of Component (A) and Components (B) and/or (C); and 30 wt-% to 70 wt-% Component (B), Component (C), or both, based on the total weight of Component (A) and Components (B) and/or (C).

In certain embodiments of the present disclosure, a treating composition includes Component (B). In certain embodiments, a treating composition includes Component (B) and at least one of Component (A) and Component (C).

Certain embodiments of compositions (e.g., fluorine-free treating compositions) of the present disclosure include: from 1 wt-% to 99 wt-% of Component (B), based on the total weight of Component (B) and Components (A) and/or (C); and from 1 wt-% to 99 wt-% of Component (A), Component (C), or both, based on the total weight of Component (B) and Components (A) and/or (C).

Certain embodiments of compositions (e.g., fluorine-free treating compositions) of the present disclosure include: 10 wt-% to 90 wt-% of Component (B), based on the total weight of Component (B) and Components (A) and/or (C); and 10 wt-% to 90 wt-% of Component (A), Component (C), or both, based on the total weight of Component (B) and Components (A) and/or (C).

Certain embodiments of compositions (e.g., fluorine-free treating compositions) of the present disclosure include: 20 wt-% to 80 wt-% Component (B), based on the total weight of Component (B) and Components (A) and/or (C); and 20 t-% to 80 wt-% Component (A), Component (C), or both, based on the total weight of Component (B) and Components (A) and/or (C).

Certain embodiments of compositions (e.g., fluorine-free treating compositions) of the present disclosure include: 30 wt-% to 70 wt-% Component (B), based on the total weight of Component (B) and Components (A) and/or (C); and 30 wt-% to 70 wt-% Component (A), Component (C), or both, based on the total weight of Component (B) and Components (A) and/or (C).

In certain embodiments of the present disclosure, a treating composition includes Component (C). In certain embodiments, a treating composition includes Component (C) and at least one of Component (A) and Component (B).

Certain embodiments of compositions (e.g., fluorine-free treating compositions) of the present disclosure include: from 1 wt-% to 99 wt-% of Component (C), based on the total weight of Component (C) and Components (A) and/or (B); and from 1 wt-% to 99 wt-% of Component (A), Component (B), or both, based on the total weight of Component (C) and Components (A) and/or (B).

Certain embodiments of compositions (e.g., fluorine-free treating compositions) of the present disclosure include: 10 wt-% to 90 wt-% of Component (C), based on the total weight of Component (C) and Components (A) and/or (B); and 10 wt-% to 90 wt-% of Component (A), Component (B), or both, based on the total weight of Component (C) and Components (A) and/or (B).

Certain embodiments of compositions (e.g. fluorine-free treating compositions) of the present disclosure include: 20 wt-% to 80 wt-% Component (C), based on the total weight of Component (C) and Components (A) and/or (B); and 20 wt-% to 80 wt-% Component (A), Component (B), or both, based on the total weight of Component (C) and Components (A) and/or (B).

Certain embodiments of compositions (e.g., fluorine-free treating compositions) of the present disclosure include: 30 wt-% to 70 wt-% Component (C), based on the total weight of Component (C) and Components (A) and/or (B); and 30 wt-% to 70 wt-% Component (A), Component (B), or both, based on the total weight of Component (C) and Components (A) and/or (B).

### EXEMPLARY EMBODIMENTS

### Embodiments Set 1: Methods Including Use of Component (A)

Embodiment 1 is a method of treating a fibrous substrate, the method comprising applying a fluorine-free treating composition in an amount sufficient to make the fibrous substrate water repellent, wherein the treating composition comprises:

one or more polymeric compounds derived from the polymerization of at least one monomer (which may be a (meth)acrylate or a (meth)acrylamide) of Formula (I):

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms.

Embodiment 2 is the method of embodiment 1 wherein D¹ is —NH—, and the one or more polymeric compounds comprises a homopolymer of Formula (I).

Embodiment 3 is the method of embodiment 1 or 2 wherein each L¹, L², L³, L⁴, L⁵, L⁶, L⁷, L⁸, L⁹, and L¹⁰ is independently a straight chain (i.e., linear) alkylene group having 2 to 10 carbon atoms.

Embodiment 4 is the method of embodiment 3 wherein each L¹, L², L³, L⁴, L⁵, L⁶, L⁷, L⁸, L⁹, and L¹⁰ is independently selected from an ethylene group, a butylene group, or a propylene group.

Embodiment 5 is the method of any one of embodiments 1 through 4 wherein the one or more polymeric compounds have a weight average molecular weight of greater than 20,000 Daltons and up to 500,000 Daltons.

Embodiment 6 is the method of any one of embodiments 1 through 5 wherein the one or more polymeric compounds have an average of greater than 20 repeating units of at least one monomer of Formula (I).

Embodiment 7 is the method of any one of embodiments 1 through 6 wherein R¹ of Formula (I) represents a hydrocarbon group having 16 to 60 carbon atoms.

Embodiment 8 is the method of embodiment 7 wherein R¹ of Formula (I) represents a hydrocarbon group having 16 to 30 carbon atoms.

Embodiment 9 is the method of any one of embodiments 1 and 3 through 8 (except as dependent on embodiment 2) wherein the one or more polymeric compounds additionally include units derived from a monomer with one or more functional groups capable of undergoing additional reactions.

Embodiment 10 is the method of embodiment 9 wherein the functional groups capable of undergoing additional reactions are selected from a polymerizable olefin group, an olefin group that can undergo a hydrosilation reaction, an epoxy group, a hydroxyl group, a halo group, a haloformyl group, an aziridino group, an acid group, a salt of an acid group, an amino group, a salt of an amino group, a quaternary ammonium group, a salt of a quaternary ammonium group, a blocked isocyanate group, a hydroxyalkyl group, a chlorinated hydroxyalkyl group, an N-methylol group, an acetoacetoxyalkyl group, and a combination thereof.

Embodiment 11 is the method of any one of embodiments 1 and 3 through 10 (except as dependent on embodiment 2) wherein the one or more polymeric compounds are derived from the polymerization of at least one monomer of Formula (I) and at least one monomer of Formula (II):

R³-D²-C(O)C(R⁴)═CH₂ Formula (II)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NHC(O)OL¹¹-O—;
—O—C(O)NH-L¹²-O—;
—NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

wherein the at least one monomer of Formula (II) is present in the one or more polymeric compounds in an amount of less than 30 wt-%, based on the total amount of monomers.

Embodiment 12 is the method of embodiment 11 wherein R³ of Formula (II) represents a hydrocarbon group having 16 to 60 carbon atoms.

Embodiment 13 is the method of embodiment 12 wherein R³ of Formula (I) represents a hydrocarbon group having 16 to 30 carbon atoms.

Embodiment 14 is the method of any one of embodiments 1 through 13 wherein the one or more polymeric compounds are derived from at least one monomer of the following formulas:

C₁₈H₃₇—NH—C(O)C(R²)═CH₂; and

C₁₈H₃₇—NH—C(O)—NH—C(O)—C(R²)═CH₂; and

C₁₇H₃₅—C(O)OCH₂CH₂OC(O)C(R²)═CH₂.

wherein R² represents H or CH₃.

Embodiment 15 is the method of embodiment 14 wherein the one or more polymeric compounds are derived from at least one monomer of the following formula:

C₁₈H₃₇—NH—C(O)C(R²)═CH₂

wherein R² represents H or CH₃ (preferably a homopolymer of C₁₈H₃₇—NH—C(O)C(H)═CH₂).

Embodiment 16 is the method of any one of embodiments 1 through 15 wherein the fluorine-free treating composition is an aqueous dispersion.

Embodiment 17 is the method of any one of embodiments 1 through 16 wherein the fluorine-free treating composition further comprises one or more additives selected from a paraffin wax, a surfactant, a coalescing solvent, an anti-freeze solvent, an emulsifier, and a stabilizer against one or more microorganisms.

Embodiment 18 is the method of any one of embodiments 1 through 17 wherein applying the composition to a fibrous substrate comprises applying the composition in an amount sufficient to make the fibrous substrate durably water repellent.

Embodiment 19 is a fibrous substrate treated by the method of any one of embodiments 1 through 18.

Embodiment 20 is the fibrous substrate of embodiment 19 which is selected from the group of textile, leather, carpet, paper, and fabrics.

### Embodiments Set II: Component (A)—Containing Blends and Uses

Embodiment 1 is a fluorine-free treating composition comprising Component (A) and at least one of Component (B) and Component (C), wherein:

Component (A) comprises one or more polymeric compounds derived from the polymerization of at least one monomer (which may be a (meth)acrylate or a (meth)acrylamide) of Formula (I):

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms;

Component (B) comprises:

one or more compounds derived from reaction, in one or more steps, of components comprising:
(i) at least one isocyanate-reactive oligomer comprising 2 to 20 repeating units, or an alcohol, anine, acid, amide, or thiol, having at least one linear or branched hydrocarbon group having from 4 to 60 carbon atoms (16 to 60 carbon atoms, 16 to 50 carbon atoms, 16 to 40 carbon atoms, or 16 to 30 carbon atoms) and optionally one or more ester groups (in certain embodiments, at least one isocyanate-reactive oligomer comprising 2 to 20 repeating units);
(ii) at least one polyisocyanate;
(iii) optionally at least one additional mono-, di-, or poly-functional isocyanate-reactive compound; and
(iv) optionally at least one isocyanate blocking agent;

wherein the isocyanate-reactive oligomer is made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms); and

Component (C) comprises one or more polycarbodiimide compounds.

Embodiment 2 is the fluorine-free treating composition of embodiment 1 comprising: from 1 wt-% to 99 wt-% of Component (A), based on the total weight of Component (A) and Components (B) and/or (C); and from 1 wt-% to 99 wt-% of Component (B), Component (C), or both, based on the total weight of Component (A) and Components (B) and/or (C).

Embodiment 3 is the fluorine-free treating composition of embodiment 2 comprising: 10 wt-% to 90 wt-% of Component (A), based on the total weight of Component (A) and Components (B) and/or (C); and 10 wt-% to 90 wt-% of Component (B), Component (C), or both, based on the total weight of Component (A) and Components (B) and/or (C).

Embodiment 4 is the fluorine-free treating composition of embodiment 3 comprising: 20 wt-% to 80 wt-% Component (A), based on the total weight of Component (A) and Components (B) and/or (C); and 20 wt-% to 80 wt-% Component (B), Component (C), or both, based on the total weight of Component (A) and Components(B) and/or (C).

Embodiment 5 is the fluorine-free treating composition of embodiment 4 comprising: 30 wt-% to 70 wt-% Component (A), based on the total weight of Component (A) and Components (B) and/or (C); and 30 wt-% to 70 wt-% Component (B), Component (C), or both, based on the total weight of Component (A) and Components (B) and/or (C).

Embodiment 6 is the fluorine-free treating composition of embodiment 1 wherein, in Component (A), D¹ is —NH—, and the one or more polymeric compounds comprise a homopolymer of Formula (I).

Embodiment 7 is the fluorine-free treating composition of any one of embodiments 1 through 6 wherein, in Component (A), each L¹, L², L³, L⁴, L⁵, L⁶, L⁷, L⁸, L⁹, and L¹⁰ is independently a straight chain (i.e., linear) alkylene group having 2 to 10 carbon atoms (in certain embodiments, an ethylene group, a butylene group, or a propylene group).

Embodiment 8 is the fluorine-free treating composition of any one of embodiments 1 through 7 wherein, in Component (A), the one or more polymeric compounds have a weight average molecular weight of greater than 20,000 Daltons and up to 500,000 Daltons.

Embodiment 9 is the fluorine-free treating composition of any one of embodiments 1 through 8 wherein, in Component (A), the one or more polymeric compounds have an average of greater than 20 repeating units of at least one monomer of Formula (I).

Embodiment 10 is the fluorine-free treating composition of any one of embodiments 1 through 9 wherein, in Component (A), the one or more polymeric compounds additionally include units derived from a monomer with one or more functional groups capable of undergoing additional reactions.

Embodiment 11 is the fluorine-free treating composition of embodiment 10 wherein the functional groups capable of undergoing additional reactions are selected from a polymerizable olefin group, an olefin group that can undergo a hydrosilation reaction, an epoxy group, a hydroxyl group, a halo group, a haloformyl group, an aziridino group, an acid group, a salt of an acid group, an amino group, a salt of an amino group, a quaternary ammonium group, a salt of a quaternary ammonium group, a blocked isocyanate group, a hydroxyalkyl group, a chlorinated hydroxyalkyl group, an N-methylol group, an acetoacetoxyalkyl group, and a combination thereof.

Embodiment 12 is the fluorine-free treating composition of any one of embodiments 1 through 11 wherein, in Component (A), the one or more polymeric compounds are derived from the polymerization of at least one monomer of Formula (I) and at least one monomer of Formula (II):

R³-D²-C(O)C(R⁴)═CH₂ Formula (II)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NHC(O)OL¹¹-O—;
—O—C(O)NH-L¹²-O—;
—NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

wherein the at least one monomer of Formula (II) is present in the one or more polymeric compounds in an amount of less than 30 wt-%, based on the total amount of monomers.

Embodiment 13 is the fluorine-free treating composition of embodiment 12 wherein the one or more polymeric compounds are derived from at least one monomer of the following formulas:

C₁₈H₃₇—NH—C(O)C(R²)═CH₂;

C₁₈H₃₇—NH—C(O)—NH—C(O)—C(R²)═CH₂; and

C₁₇H₃₅C(O)OCH₂CH₂OC(O)C(R²)═CH₂.

wherein R² represents H or CH₃.

Embodiment 14 is the composition of embodiment 13 wherein the one or more polymeric compounds are derived from at least one monomer of the following formula:

C₁₈H₃₇—NH—C(O)C(R²)═CH₂

wherein R² represents H or CH (preferably a homopolymer of C₁₈H₃₇—NH—C(O)C(H)═CH₂).

Embodiment 15 is the fluorine-free treating composition of any one of embodiments 1 through 14 wherein the components for making the one or more compounds of Component (B) comprise at least one additional mono-, di-, or poly-functional isocyanate-reactive compound.

Embodiment 16 is the fluorine-free treating composition of embodiment 15 wherein the additional mono-, di-, or poly-functional isocyanate-reactive compound of Component (B) comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms; or a combination thereof.

Embodiment 17 is the fluorine-free treating composition of embodiment 16 wherein the additional mono-, di-, or poly-functional isocyanate-reactive compound of Component (B) comprises a mono-, di-, or poly-functional alcohol, thiol, amine, acid, or amide.

Embodiment 18 is the fluorine-free treating composition of any one of embodiments 1 through 17 wherein the components for making the one or more compounds of Component (B) comprises a blocked isocyanate group.

Embodiment 19 is the fluorine-free treating composition of embodiment 18 wherein the blocked isocyanate group is an oxime-derived group.

Embodiment 20 is the fluorine-free treating composition of any one of embodiments 1 through 19 wherein the isocyanate-reactive oligomer for making the one or more compounds of Component (B) are made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms), and at least one isocyanate-derived group.

Embodiment 21 is the fluorine-free treating composition of any one of embodiments 1 through 20 wherein the one or more polycarbodiimide compounds of Component (C) are derived from a carbodiimidization reaction, in one or more steps, of components comprising: (i) at least one isocyanate-reactive oligomer (i.e., functionalized oligomer) comprising 2 to 20 repeating units, or an alcohol, amine, acid, amide, or thiol, having at least one linear or branched hydrocarbon group having from 4 to 60 carbon atoms (16 to 60 carbon atoms, 16 to 50 carbon atoms, 16 to 40 carbon atoms, or 16 to 30 carbon atoms) and optionally one or more ester groups (in certain embodiments, at least one isocyanate-reactive oligomer comprising 2 to 20 repeating units, or an amine, acid, or amide having at least one linear or branched hydrocarbon group having from 4 to 60 carbon atoms and optionally one or more ester groups, and in certain embodiments, at least one isocyanate-reactive oligomer comprising 2 to 20 repeating units); (ii) at least one polyisocyanate (which may be an aromatic polyisocyanate); and (iii) optionally at least one additional mono-, di-, or poly-functional isocyanate-reactive compound; wherein the isocyanate-reactive oligomer is made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms).

Embodiment 22 is the fluorine-free treating composition of embodiment 21 wherein the isocyanate-reactive oligomer for making the one or more polycarbodiimide compounds of Component (C) are made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms), and at least one isocyanate-derived group.

Embodiment 23 is the fluorine-free treating composition of any one of embodiments 1 through 22 wherein the monomer for making the isocyanate-reactive oligomers used to make the one or more compounds of Component (B) and/or Component (C) has at least one of the following formulas:

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)U—C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms;

R³-D²-C(O)C(R⁴)═CH₂ Formula (II)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NHC(O)OL¹¹-O—;
—O—C(O)NH-L¹²-O—;
—NHC(O)NH-L¹³—O—; and
—O—; and

each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)=CH₂Formula(III) or

R⁷—X²—C(O)NH-Q¹-NH—C(O)O-L¹⁵-OC(O)C(R⁸)═CH₂ Formula (IV)

wherein:

R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);

R⁶ and R⁸ are independently H or CH₃;

L¹⁴ and L¹⁵ are independently a branched or straight chain alkylene group having 2 to 10 carbon atoms, an arylene group (in some embodiments, an arylene group having 5 to 12 carbon atoms), or a combination thereof:

X¹ is S or —N(R⁹) and X² is O, S, —NH, or —N(R⁹), wherein R is a hydrocarbon group (in certain embodiments, a linear or branched hydrocarbon group, and in certain embodiments, a linear hydrocarbon group) having 1 to 20 carbon atoms; and
Q¹ is a divalent isocyanate residue.

Embodiment 24 is the fluorine-free treating composition of any one of embodiments 1 through 23 wherein the isocyanate-reactive oligomer used to make the one or more compounds of Component (B) and/or Component (C) are made by the oligomerization of at least one (meth)acrylate or (meth)acrylamide monomer in the presence of at least one mercaptan (which may or may not be functionalized), wherein the molar ratio of the mercaptan to the (meth)acrylate or (meth)acrylamide monomer is 1:4 to 1:20 (or, in certain embodiments, 1:8 to 1:16).

Embodiment 25 is the fluorine-free treating composition of any one of embodiments 21 through 24 wherein the components for making the one or more compounds of Component (C) comprise at least one additional mono-, di-, or poly-functional isocyanate-reactive compound.

Embodiment 26 is the fluorine-free treating composition of embodiment 25 wherein the additional mono-, di-, or poly-functional isocyanate-reactive compound of Component (C) comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms; or a combination thereof.

Embodiment 27 is the fluorine-free treating composition of embodiment 26 wherein the additional mono-, di-, or poly-functional isocyanate-reactive compound of Component (C) comprises a mono-, di-, or poly-functional alcohol, thiol, amine, acid, or amide.

Embodiment 28 is the fluorine-free treating composition of any one of embodiments 1 through 27 wherein the one or more polycarbodiimide compounds of Component (C) have the following formula:

Q²(X³—C(O)NH-(A¹-(N═C═N))ᵣ-A²-NHC(O)—X⁴)ₛ-Q³ Formula (VII)

wherein:

X³ is a chemical bond, —O—, —NH—, —N(R¹⁴), —S— or —C(O)NH—, and X⁴ is a chemical bond, —O—, —N—, —N(R), —S— or —NHC(O)—, wherein R¹⁴ is a hydrocarbon group (e.g. an alkyl group) having 1 to 20 carbon atoms (e.g., methyl, ethyl, octyl, and octadecyl);

A¹ and A² each represents the residue of an organic di-isocyanate compound;

s is 1 or 2, preferably s is 1;

r is 2 to 10, preferably r is 3; and

Q² and Q³ are independently selected from: a hydrocarbon group (in certain embodiments, a linear or branched hydrocarbon group) having at least 2 carbon atoms; and a group having the formula:

—(CH₂)ₐ—S—U Formula (VIII)
wherein:
   a is an integer from 1 to 10;
   S is sulfur; and
   U is an oligomeric group comprising 2 to 20 repeating units of monomers selected from at least one of Formulas (I), (II), (III), and (IV):

R¹-D¹-C(O)C(R²)═CH₂ Formula (I)
wherein:
   R¹ represents a hydrocarbon group having 4 to 60 carbon atoms (16 to 60 carbon atoms, 16 to 50 carbon atoms, 16 to 40 carbon atoms, or 16 to 30 carbon atoms); and
   R² represents H or CH₃;
   D¹ is selected from:
      —C(O)O-L¹-O—;
      —OC(O)-L²-O—;
   —OC(O)-L³-C(O)O-L⁴-O—;
   —C(O)NH-L⁵-O—;
   —NHC(O)-L⁶-O—;
   —NHC(O)-L⁷-C(O)O-L⁸-O—;
   —C(O)—N—;
   —C(O)NHC(O)NH-L⁹-O—;
   —C(O)NHC(O)-L¹⁰-O—;
   —NHC(O)NH—; and
   —NH—; with the proviso that when D¹ is —NH—,the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms; and

R³-D²-C(O)C(R⁴)═CH₂ Formula (I)
wherein:
   R³ represents a hydrocarbon group having 4 to 60 carbon atoms (16 to 60 carbon atoms, 16 to 50 carbon atoms, 16 to 40 carbon atoms, or 16 to 30 carbon atoms); and
   R⁴ represents 1- or CH₃;
   D² is selected from:
      —NHC(O)OL¹¹-O—;
      —O—C(O)NH-L¹²-O—;
   —NHC(O)NH-L¹³-O—; and
   —O—; and
each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)═CH₂ Formula (III) and

R⁷—X²—C(O)NH-Q¹-NH—C(O)O-L¹⁵-OC(O)C(R⁸)═CH₂ Formula (IV)
wherein:
   R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);
   R⁶ and R⁸ are independently H or CH₃;
   L¹⁴ and L¹⁵ are independently a branched or straight chain alkylene group having 2 to 10 carbon atoms, an arylene group (in some embodiments, an arylene group having 5 to 12 carbon atoms), or a combination thereof;
   X¹ is S or —N(R⁹) and X² is O, S, —NH, or —N(R⁹), wherein R⁹ is a hydrocarbon group (in certain embodiments, a linear or branched hydrocarbon group, and in certain embodiments, a linear hydrocarbon group) having 1 to 20 carbon atoms; and
   Q¹ is a divalent isocyanate residue.

Embodiment 29 is the fluorine-free treating composition of embodiment 28 wherein Q² and Q³ are independently selected from a hydrocarbon group (in certain embodiments, a linear or branched hydrocarbon group) having 2 to 60 carbon atoms.

Embodiment 30 is the fluorine-free treating composition of any one of embodiments 1 through 29 which is an aqueous dispersion optionally comprising one or more additives selected from a paraffin wax, a surfactant, a coalescing solvent, an anti-freeze solvent, an emulsifier, and a stabilizer against one or more microorganisms.

Embodiment 31 is a method of treating a fibrous substrate, the method comprising applying a fluorine-free treating composition of any one of embodiments 1 through 30 in an amount sufficient to make the fibrous substrate water repellent.

Embodiment 32 is the method of embodiment 31 comprising applying the fluorine-free treating composition in an amount sufficient to make the fibrous substrate durably water repellent.

Embodiment 33 is a fibrous substrate treated by the method of embodiment 31 or 32.

Embodiment 34 is the fibrous substrate of embodiment 33 which is selected from the group of textile, leather, carpet, paper, and fabrics.

### Embodiments Set III: Component (B)-Containing Compositions

Embodiment 1 is a composition comprising Component (B), wherein:

Component (B) comprises one or more compounds derived from reaction, carried out in one or more steps, of components comprising:
(i) at least one:
   isocyanate-reactive (i.e., functionalized) oligomer comprising 2 to 20 repeating units; or
   an amine, acid, or amide, having at least one linear or branched hydrocarbon group having from 4 to 60 carbon atoms and optionally one or more ester groups;
(ii) at least one polyisocyanate;
(iii) optionally at least one additional mono-, di- or poly-functional isocyanate-reactive compound; and
(iv) optionally at least one isocyanate blocking agent;

with the proviso that less than 40% of the isocyanate groups are reacted with acid and/or amide groups;

wherein the isocyanate-reactive oligomer has the following formula:

Y¹[CH₂—C(R²)C(O)-D¹-R¹]ₘ—S—R¹⁰-(T¹)ₚ Formula (V)
wherein:
   Y¹ is H or an initiator residue;
   R¹ is a hydrocarbon group having 4 to 60 carbon atoms (16 to 60 carbon atoms, 16 to 50 carbon atoms, 16 to 40 carbon atoms, or 16 to 30 carbon atoms);
   R² is independently H or CH₃;
   D¹ is selected from:
      —C(O)O-L¹-O—;
      —OC(O)-L²-O—;
      —OC(O)-L³-C(O)O-L⁴-O—;
      —C(O)NH-L⁵-O—;
      —NHC(O)-L⁶-O—;
      NHC(O)-L⁷-C(O)O-L⁸-O—;
      —C(O)—NH—;
      —C(O)NHC(O)NH-L⁹-O—;
   —C(O)NHC(O)-L¹⁰-O—;
   —NHC(O)NH—; and
   —NH—; with the proviso that when D¹ is —NH—, the isocyanate-reactive oligomer comprises greater than 30 wt-% of monomeric units that include —NH—, based on the total weight of monomeric units;
   each L¹, L², L³, L⁴, L⁵, L⁶, L⁷, L⁸, L⁹, and L¹⁰ is independently a branched or straight chain alkylene group having 2 to 10 carbon atoms;
   S is sulfur;
   R¹⁰ is a divalent or trivalent linking group having 1 to 10 carbon atoms;
   T¹ is —C(O)OH, —C(O)NH₂, —OH, —NH₂, or —NH(R¹¹), wherein R¹¹ is H or a hydrocarbon group having 1 to 10 carbon atoms;
   m is an integer of 2 to 20; and
   p is independently 1 or 2;
      and/or

Component (B) comprises one or more compounds derived from reaction, carried out in one or more steps, of components comprising:
(i) at least one:
   isocyanate-reactive (i.e., functionalized) oligomer comprising 2 to 20 repeating units; or
   an amine, acid, or amide, having at least one linear or branched hydrocarbon group having from 4 to 60 carbon atoms and optionally one or more ester groups;
(ii) at least one polyisocyanate;
(iii) optionally at least one additional mono-, di- or poly-functional isocyanate-reactive compound; and
(iv) optionally at least one isocyanate blocking agent;

wherein the isocyanate-reactive oligomer has the following formula:

Y²—[CH₂—C(R⁴)C(O)-D²-R³]ₘ—S—R¹²-(T²)ₚ Formula (VI)
wherein:
   Y² is H or an initiator residue;
   R³ is a hydrocarbon group having 4 to 60 carbon atoms (16 to 60 carbon atoms, 16 to 50 carbon atoms, 16 to 40 carbon atoms, or 16 to 30 carbon atoms);
   R⁴ is independently H or CH₃;
   D² is selected from:
      —NHC(O)OL¹¹-O—;
      —O—C(O)NH-L¹²-O—;
      —NHC(O)NH-L¹³-O—; and
      —O—; and
   each L¹¹, L¹², and L¹³ is independently a branched or straight chain alkylene group having 2 to 10 carbon atoms;
   S is sulfur;
   R¹² is a divalent or trivalent linking group having 1 to 10 carbon atoms;
   T² is —C(O)OH, —C(O)NH₂, —O—, NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
   m is an integer of 2 to 20; and
   p is independently 1 or 2.

Embodiment 2 is the composition of embodiment 1 wherein D¹ is —NH—, and the oligomer includes 100% of monomeric units that include —NH—.

Embodiment 3 is the composition of embodiment 1 or 2 wherein each L¹, L², L³, L⁴, L⁵, L⁶, L⁷, L⁸, L⁹, and L¹⁰ is independently a straight chain (i.e., linear) alkylene group having 2 to 10 carbon atoms.

Embodiment 4 is the composition of any one of embodiments 1 through 3 wherein each L¹¹, L¹², and L¹³ is independently a straight chain (i.e., linear) alkylene group having 2 to 10 carbon atoms.

Embodiment 5 is the composition of any one of embodiments 1 through 4 wherein the at least one polyisocyanate comprises an aromatic diisocyanate, an aromatic triisocyanate, an aromatic polymeric isocyanate, or a mixture thereof.

Embodiment 6 is the composition of any one of embodiments 1 through 5 wherein the components for making the one or more compounds of Component (B) comprise at least one additional mono-, di-, or poly-functional isocyanate-reactive compound.

Embodiment 7 is the composition of embodiment 6 wherein the additional mono-, di-, or poly-functional isocyanate-reactive compound of Component (B) comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms; or a combination thereof.

Embodiment 8 is the composition of embodiment 7 wherein the additional mono-, di-, or poly-functional isocyanate-reactive compound of Component (B) comprises a mono- di-, or poly-functional alcohol, thiol, amine, acid, or amide.

Embodiment 9 is the composition of any one of embodiments 1 through 8 wherein the components for making the one or more compounds of Component (B) comprises a blocked isocyanate group (e.g., an oxime-derived group).

Embodiment 10 is the composition of any one of embodiments 1 through 9 further comprising at least one of Component (A) and Component(C) (in certain embodiments, Component (A) and optionally Component (C)), wherein: Component (A) comprises one or more polymeric compounds derived from the polymerization of at least one monomer comprising at least one hydrocarbon group having from 4 to 60 carbon atoms (16 to 60 carbon atoms, 16 to 50 carbon atoms, 16 to 40 carbon atoms, or 16 to 30 carbon atoms); and Component (C) comprises one or more polycarbodiimide compounds.

Embodiment 11 is the composition of embodiment 10 comprising: from 1 wt-% to 99 wt-% of Component (B), based on the total weight of Component (B) and Components (A) and/or (C); and from 1 wt-% to 99 wt-% of Component (A), Component (C), or both, based on the total weight of Component (B) and Components (A) and/or (C).

Embodiment 12 is the composition of embodiment 1l comprising: 10 wt-% to 90 wt-% of Component (B), based on the total weight of Component (B) and Components (A) and/or (C); and 10 wt-% to 90 wt-% of Component (A), Component (C), or both, based on the total weight of Component (B) and Components (A) and/or (C).

Embodiment 13 is the composition of embodiment 12 comprising: 20 wt-% to 80 wt-% Component (B), based on the total weight of Component (B) and Components (A) and/or (C); and 20 wt-% to 80 wt-% Component (A), Component (C), or both, based on the total weight of Component (B) and Components (A) and/or (C).

Embodiment 14 is the composition of embodiment 13 comprising: 30 wt-% to 70 wt-% Component (B), based on the total weight of Component (B) and Components (A) and/or (C); and 30 wt-% to 70 wt-% Component (A), Component (C), or both, based on the total weight of Component (B) and Components (A) and/or (C).

Embodiment 15 is the composition of any one of embodiments 10 through 14 wherein the at least one monomer for making the one or more compounds of Component (A) has at least one of the following formulas:

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms;

R³-D²-C(O)C(R⁴)═CH₂ Formula (II)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NHC(O)OL¹¹-O—;
—O—C(O)NH-L¹²-O—;
—NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)═CH₂ Formula (III) or

R⁷—X²—C(O)NH-Q¹-NH—C(O)O-L¹⁵-OC(O)C(R⁸)═CH₂ Formula (IV)

wherein:
R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);
R⁶ and R⁸ are independently H or CH₃;
L¹⁴ and L¹⁵ are independently a branched or straight chain alkylene group having 2 to 10 carbon atoms, an arylene group (in some embodiments, an arylene group having 5 to 12 carbon atoms), or a combination thereof;
X¹ is S or —N(R⁹) and X² is O, S, —NH, or —N(R⁹), wherein R is a hydrocarbon group (in certain embodiments, a linear or branched hydrocarbon group, and in certain embodiments, a linear hydrocarbon group) having 1 to 20 carbon atoms; and
Q¹ is a divalent isocyanate residue.

Embodiment 16 is the composition of embodiment 15 wherein the one or more compounds of Component (A) are derived from the polymerization of at least one monomer of Formula (I).

Embodiment 17 is the composition of embodiment 16 wherein D¹ is —NH—, and the one or more polymeric compounds comprise a homopolymer of Formula (I).

Embodiment 18 is the composition of embodiment 16 or 17 wherein, in Component (A), each L¹, L², L³, L⁴, L⁵, L⁶, L⁷, L⁸, L⁹, and L¹⁰ is independently a straight chain (i.e., linear) alkylene group having 2 to 10 carbon atoms (in certain embodiments, an ethylene group, a butylene group, or a propylene group).

Embodiment 19 is the composition of any one of embodiments 15 through 18 wherein, in Component (A), the one or more polymeric compounds are derived from the polymerization of at least one monomer of Formula (I) and at least one monomer of Formula (II).

Embodiment 20 is the composition of any one of embodiments 15 through 19 wherein, in Component (A), the one or more polymeric compounds additionally include units derived from a monomer with one or more functional groups capable of undergoing additional reactions.

Embodiment 21 is the composition of embodiment 20 wherein the functional groups capable of undergoing additional reactions are selected from a polymerizable olefin group, an olefin group that can undergo a hydrosilation reaction, an epoxy group, a hydroxyl group, a halo group, a haloformyl group, an aziridino group, an acid group, a salt of an acid group, an amino group, a salt of an amino group, a quaternary ammonium group, a salt of a quaternary ammonium group, a blocked isocyanate group, a hydroxyalkyl group, a chlorinated hydroxyalkyl group, an N-methylol group, an acetoacetoxyalkyl group, and a combination thereof.

Embodiment 22 is the composition of any one of embodiments 15 through 21 wherein the one or more polymeric compounds are derived from at least one monomer of the following formulas:

C₁₈H₃₇—NH—C(O)C(R²)═CH₂;

C₁₈H₃₇ —NH—C(O)OCH₂CH₂OC(O)C(R²)═CH₂;

C₁₈H₃₇—NH—C(O)—NH—C(O)—C(R²)═CH₂; and

C₁₇H₃₇—C(O)OCH₂CH₂OC(O)C(R²)═CH₂.

wherein R² represents H or CH₃.

Embodiment 23 is the composition of embodiment 22 wherein the one or more polymeric compounds are derived from at least one monomer of the following formula:

C₁₈H₃₇—NH—C(O)C(R²)═CH₂

wherein R² represents H or CH₃ (preferably a homopolymer of C₁₈H₃₇—NH—C(O)C(H)═CH₂).

Embodiment 24 is the composition of any one of embodiments 10 through 23 wherein, in Component (A), the one or more polymeric compounds have a weight average molecular weight of greater than 20,000 Daltons and up to 500,000 Daltons.

Embodiment 25 is the composition of any one of embodiments 10 through 24 wherein, in Component (A), the one or more polymeric compounds have an average of greater than 20 repeating units of at least one monomer (preferably, at least one monomer of Formula (I)).

Embodiment 26 is the composition of any one of embodiments 10 through 25 wherein the one or more polycarbodiimide compounds of Component (C) are derived from a carbodiimidization reaction, in one or more steps, of components comprising:

(i) at least one:
isocyanate-reactive oligomer (i.e., functionalized oligomer) comprising 2 to 20 repeating units; or
an alcohol, amine, acid, amide, or thiol, having at least one linear or branched hydrocarbon group having from 4 to 60 carbon atoms (16 to 60 carbon atoms, 16 to 50 carbon atoms, 16 to 40 carbon atoms, 16 to 30 carbon atoms) and optionally one or more ester groups;

(ii) at least one polyisocyanate (which may be an aromatic polyisocyanate); and

(iii) optionally at least one additional mono-, di-, or poly-functional isocyanate-reactive compound;

wherein the isocyanate-reactive oligomer is made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms).

Embodiment 27 is the composition of embodiment 26 wherein the isocyanate-reactive oligomer for making the one or more polycarbodiimide compounds of Component (C) are made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms), and at least one isocyanate-derived group.

Embodiment 28 is the composition of embodiment 26 or 27 wherein the monomer for making the isocyanate-reactive oligomers used to make the one or more compounds of Component (C) has at least one of the following formulas:

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms;

R³-D²-C(O)C(R⁴)═CH₂ Formula (II)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NHC(O)OL¹¹-O—;
—O—C(O)NH-L¹²-O—;
—NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)═CH₂ Formula (III) or

R⁷—X²—C(O)NH-Q¹-NH—C(O)O-L¹⁵-OC(O)C(R⁸)═CH₂ Formula (IV)

wherein:
R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);
R⁶ and R⁸ are independently H or CH₃;
L¹⁴ and L¹⁵ are independently a branched or straight chain alkylene group having 2 to 10 carbon atoms, an arylene group (in some embodiments, an arylene group having 5 to 12 carbon atoms), or a combination thereof;
X¹ is S or —N(R⁹) and X² is O, S, —NH, or —N(R⁹), wherein R⁹ is a hydrocarbon group (in certain embodiments, a linear or branched hydrocarbon group, and in certain embodiments, a linear hydrocarbon group) having 1 to 20 carbon atoms; and
Q¹ is a divalent isocyanate residue.

Embodiment 29 is the composition of any one of embodiments 26 through 28 wherein the isocyanate-reactive oligomer used to make the one or more compounds of Component (C) are made by the oligomerization of at least one (meth)acrylate or (meth)acrylamide monomer in the presence of at least one mercaptan (which may or may not be functionalized), wherein the molar ratio of the mercaptan to the (meth)acrylate or (meth(acrylamide monomer is 1:4 to 1:20 (or, in certain embodiments, 1:8 to 1:16).

Embodiment 30 is the composition of anyone of embodiments 26 through 29 wherein the components for making the one or more compounds of Component (C) comprise at least one additional mono-, di-, or poly-functional isocyanate-reactive compound.

Embodiment 31 is the composition of embodiment 30 wherein the additional mono-, di-, or poly-functional isocyanate-reactive compound of Component (C) comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms or a combination thereof.

Embodiment 32 is the composition of embodiment 31 wherein the additional mono-, di-, or poly-functional isocyanate-reactive compound of Component (C) comprises a mono-, di-, or poly-functional alcohol, thiol, amine, acid, or amide.

Embodiment 33 is the composition of any one of embodiments 26 through 32 wherein the one or more polycarbodiimide compounds of Component (C) have the following formula:

Q²-(X³—C(O)NH-(A¹-(N═C═N))ᵣ-A²-NHC(O)—X⁴)ₛ-Q³ Formula (VII)

wherein:
X³ is a chemical bond, —O—, —NH—, —N(R¹⁴), —S—, or —C(O)NH—, and X⁴ is a chemical bond, —O—, —NH—, —N(R¹⁴), —S—, or —NHC(O)—, wherein R¹⁴ is a hydrocarbon group (e.g., an alkyl group) having 1 to 20 carbon atoms (e.g., methyl, ethyl, octyl, and octadecyl);
A¹ and A² each represents the residue of an organic di-isocyanate compound;
s is 1 or 2, preferably s is 1;
r is 2 to 10, preferably r is 3; and
Q² and Q³ are independently selected from: a hydrocarbon group (in certain embodiments, a linear or branched hydrocarbon group) having at least 2 carbon atoms; and a group having the formula:

—(CH₂)ₐ—S—U Formula (VIII)
wherein:
   a is an integer from 1 to 10;
   S is sulfur; and
   U is an oligomeric group comprising 2 to 20 repeating units of monomers, according to at least one of formulas:

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms;

R³-D²-C(O)C(R⁴)═CH₂ Formula (I)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—N—C(O)OL¹¹-O—;
—O—C(O)NH-L¹²-O—;
—NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)═CH₂ Formula (III) or

R⁷—X²—C(O)NH-Q¹-NH—C(O)O-L¹⁵-OC(O)C(R⁸)═CH₂ Formula (IV)

wherein:

R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);

R⁶ and R⁸ are independently H or CH₃;

L¹⁴ and L¹⁵ are independently a branched or straight chain alkylene group having 2 to 10 carbon atoms, an arylene group (in some embodiments, an arylene group having 5 to 12 carbon atoms), or a combination thereof:

X¹ is S or —N(R⁹) and X² is O, S, —NH—, or —N(R⁹), wherein R⁹ is a hydrocarbon group (in certain embodiments, a linear or branched hydrocarbon group, and in certain embodiments, a linear hydrocarbon group) having 1 to 20 carbon atoms; and

Q¹ is a divalent isocyanate residue

Embodiment 34 is the composition of embodiment 33 wherein Q² and Q are independently selected from a hydrocarbon group (in certain embodiments, a linear or branched hydrocarbon group) having 2 to 60 carbon atoms.

Embodiment 35 is the composition of any one of embodiments 1 through 34 which is an aqueous dispersion optionally comprising one or more additives selected from a paraffin wax, a surfactant, a coalescing solvent, an anti-freeze solvent, an emulsifier, and a stabilizer against one or more microorganisms.

Embodiment 36 is the composition of any one of embodiments 1 through 35 which is a treating composition.

Embodiment 37 is the composition of embodiment 36 which is a fluorine-free treating composition.

Embodiment 38 is a method of treating a fibrous substrate, the method comprising applying a treating composition of embodiment 36 or 37 in an amount sufficient to make the fibrous substrate water repellent.

Embodiment 39 is the method of embodiment 38 comprising applying the composition to make the fibrous substrate durably water repellent.

Embodiment 40 is a fibrous substrate treated by the method of embodiment 39.

Embodiment 41 is the fibrous substrate of embodiment 40 which is selected from the group of textile, leather, carpet, paper, and fabrics.

### Embodiments Set IV: Component (C)-Containing Compositions and Uses

Embodiment 1 is a composition comprising Component (C), wherein:

Component (C) comprises at least one polycarbodiimide compound derived from a carbodiimidization reaction, carried out in one or more steps, of components comprising:
(i) at least one:
   isocyanate-reactive (i.e., functionalized) oligomer comprising 2 to 20 repeating units; or
   an amine, acid, or amide, having at least one linear or branched hydrocarbon group having from 4 to 60 carbon atoms (16 to 60 carbon atoms, 16 to 50 carbon atoms, 16 to 40 carbon atoms, 16 to 30 carbon atoms) and optionally one or more ester groups;
(ii) at least one polyisocyanate; and
(iii) optionally at least one additional mono-, di- or poly-functional isocyanate-reactive compound;

wherein the isocyanate-reactive oligomer has the following formula:

Y¹—[CH₂C(R²)C(O)-D¹-R¹]ₘ—S—R¹⁰-(T¹)ₚ Formula (V)
wherein:
   Y¹ is H or an initiator residue;
   R¹ is a hydrocarbon group having 4 to 60 carbon atoms (16 to 60 carbon atoms, 16 to 50 carbon atoms, 16 to 40 carbon atoms, or 16 to 30 carbon atoms);
   R² is independently H or CH₃;
   D¹ is selected from:
      —C(O)O-L¹-O—;
      —OC(O)-L²-O—;
      —OC(O)-L³-C(O)O-L⁴-O—;
      —C(O)NH-L⁵-O—;
      —NHC(O)-L⁶-O—;
      —NHC(O)-L⁷-C(O)O-L⁸-O—;
      —C(O)—NH—;
      —C(O)NHC(O)NH-L⁹-O—;
      —C(O)NHC(O)-L¹⁰-O
      NHC(O)NH—; and
      —NH—; with the proviso that when D¹ is —NH— the isocyanate-reactive oligomer comprises greater than 30 wt-% of monomeric units that include —NH—, based on the total weight of monomeric units (preferably, when D¹ is —NH—, the oligomer includes 100% of monomeric units that include —NH—):
   each L¹, L², L³, L⁴, L⁵, L⁶, L⁷, L⁸, L⁹, and L¹⁰ is independently a branched or straight chain alkylene group (in certain embodiments, a straight chain, i.e., linear, alkylene group) having 2 to 10 carbon atoms;
   S is sulfur;
   R¹⁰ is a divalent or trivalent linking group having 1 to 10 carbon atoms;
   T¹ is —C(O)OH, —C(O)NH₂, —OH—, —NH₂, or —NH(R¹¹), wherein R¹¹ is H or a hydrocarbon group having 1 to 10 carbon atoms;
   m is an integer of 2 to 20; and
   p is independently 1 or 2;
      and/or

Component (C) comprises at least one polycarbodiimide compound derived from a carbodiimidization reaction, carried out in one or more steps, of components comprising:
(i) at least one:
   isocyanate-reactive (i.e., functionalized) oligomer comprising 2 to 20 repeating units; or
   an amine, acid, or amide, having at least one linear or branched hydrocarbon group having from 4 to 60 carbon atoms (16 to 60 carbon atoms, 16 to 50 carbon atoms, 16 to 40 carbon atoms, 16 to 30 carbon atoms) and optionally one or more ester groups;
(ii) at least one polyisocyanate; and
(iii) optionally at least one additional mono-, di- or poly-functional isocyanate-reactive compound;

wherein the isocyanate-reactive oligomer has the following formula:

Y²—[CH₂—C(R⁴)C(O)-D²-R³]ₘ—S—R¹²-(T²)ₚ Formula (VI)
wherein:
   Y² is H or an initiator residue;
   R³ is a hydrocarbon group having 4 to 60 carbon atoms (16 to 60 carbon atoms, 16 to 50 carbon atoms, 16 to 40 carbon atoms, or 16 to 30 carbon atoms);
   R⁴ is independently H or C₃;
   D² is selected from:
      —NHC(O)OL¹¹-O—;
      —O—C(O)NH-L¹²-O—;
      —NHC(O)NH-L¹³-O—; and
      —O—; and
   each L¹¹, L¹², and L¹³ is independently a branched or straight chain alkylene group (in certain embodiments, a straight chain, i.e., linear, alkylene group) having 2 to 10 carbon atoms;
   S is sulfur;
   R⁷ is a divalent or trivalent linking group having 1 to 10 carbon atoms;
   T² is —C(O)OH, —C(O)NH₂, —OH, NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
   m is an integer of 2 to 20; and
   p is independently 1 or 2.

Embodiment 2 is the composition of embodiment 1 wherein, in Component (C), the polyisocyanate comprise an aromatic diisocyanate, an aromatic triisocyanate, an aromatic polymeric isocyanate, or a mixture thereof.

Embodiment 3 is the composition of embodiment 1 or 2 wherein the components for making the one or more compounds of Component (C) comprise at least one additional mono-, di-, or poly-functional isocyanate-reactive compound.

Embodiment 4 is the composition of embodiment 3 wherein, in Component (C), the additional mono-, di-, or poly-functional isocyanate-reactive compound comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms; or a combination thereof.

Embodiment 5 is the composition of embodiment 4 wherein, in Component (C), the additional mono-, di- or poly-functional isocyanate-reactive compound comprises a mono-, di- or poly-functional alcohol, thiol, amine, acid, or amide.

Embodiment 6 is the composition of any one of embodiments 1 through 5 further comprising at least one of Component (A) and Component (B) (in certain embodiments, Component (A) and optionally Component (B)), wherein:

Component (A) comprises one or more polymeric compounds derived from the polymerization of at least one monomer comprising at least one hydrocarbon group having from 4 to 60 carbon atoms (16 to 60 carbon atoms, 16 to 50 carbon atoms, 16 to 40 carbon atoms, or 16 to 30 carbon atoms);

and

Component (B) comprises:

one or more compounds derived from reaction, in one or more steps, of components comprising:
(i) at least one:
   isocyanate-reactive oligomer comprising 2 to 20 repeating units;
   or an alcohol, amine, acid, amide or thiol, having at least one linear or branched hydrocarbon group having from 4 to 60 carbon atoms (16 to 60 carbon atoms, 16 to 50 carbon atoms, 16 to 40 carbon atoms, or 16 to 30 carbon atoms) and optionally one or more ester groups;
(ii) at least one polyisocyanate;
(iii) optionally at least one additional mono-, di-, or poly-functional isocyanate-reactive compound; and
(iv) optionally at least one isocyanate blocking agent;

wherein the isocyanate-reactive oligomer is made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);

Embodiment 7 is the composition of embodiment 6 comprising: from 1 wt-% to 99 wt-% of Component (C), based on the total weight of Component (C) and Components (A) and/or (B); and from 1 wt-% to 99 wt-% of Component (A), Component (B), or both, based on the total weight of Component (C) and Components (A) and/or (B).

Embodiment 8 is the composition of embodiment 7 comprising: 10 wt-% to 90 wt-% of Component (C), based on the total weight of Component (C) and Components (A) and/or (B); and 10 wt-% to 90 wt-% of Component (A), Component (B), or both, based on the total weight of Component (C) and Components (A) and/or (B).

Embodiment 9 is the composition of embodiment 8 comprising: 20 wt-% to 80 wt-% Component (C), based on the total weight of Component (C) and Components (A) and/or (B); and 20 wt-% to 80 wt-% Component (A), Component (B), or both, based on the total weight of Component (C) and Components (A) and/or (B).

Embodiment 10 is the composition of embodiment 9 comprising: 30 wt-% to 70 wt-% Component (C), based on the total weight of Component (C) and Components (A) and/or (B); and 30 wt-% to 70 wt-% Component (A), Component (B), or both, based on the total weight of Component (C) and Components (A) and/or (B).

Embodiment 11 is the composition of any one of embodiments 6 through 10 wherein the at least one monomer for making the one or more compounds of Component (A) has at least one of the following formulas:

R¹-D¹-C(O)C(R²)═CH₂ Formula (I)

wherein:

R¹ represents a hydrocarbon group having 4 to 60 carbon atoms (16 to 60 carbon atoms, 16 to 50 carbon atoms, 16 to 40 carbon atoms, or 16 to 30 carbon atoms); and

R² represents H or CH₃;

D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NC(O)-L⁷-C(O)O-L⁸-O—;
C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and

each L¹, L², L³, L⁴, L⁵, L⁶, L⁷, L⁸, L⁹, and L¹⁰ is independently a branched or straight chain alkylene group having 2 to 10 carbon atoms;

R³-D²-C(O)C(R⁴)═CH₂ Formula (I)

wherein:

R³ represents a hydrocarbon group having 4 to 60 carbon atoms (16 to 60 carbon atoms, 16 to 50 carbon atoms, 16 to 40 carbon atoms, or 16 to 30 carbon atoms); and

R⁴ represents H or CH₃;

D² is selected from:
—NC(O)OL¹¹-O—;
—O—C(O)NH-L¹²-;
NHC(O)NH-L¹³-O—; and
—O—; and

each L¹¹, L¹², and L¹³ is independently a branched or straight chain alkylene group (in certain embodiments, a straight chain, i.e., linear, alkylene group) having 2 to 10 carbon atoms;

R⁵—X¹—C(O)NH-L¹⁴-OC(O)C(R⁶)═CH₂ Formula (III) or

R⁷—X²—C(O)NH-Q¹-NH—C(O)O-L¹⁵-OC(O)C(R⁸)═CH₂ Formula (IV)

wherein:
R⁵ and R⁷ are independently a hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms);
R⁶ and R⁸ are independently H or CH₃
L¹⁴ and L¹⁵ are independently a branched or straight chain alkylene group having 2 to 10 carbon atoms, an arylene group (in some embodiments, an arylene group having 5 to 12 carbon atoms), or a combination thereof;
X¹ is S or —N(R⁹) and X² is O, S, —NH, or —N(R⁹), wherein R⁹ is a hydrocarbon group (in certain embodiments, a linear or branched hydrocarbon group, and in certain embodiments, a linear hydrocarbon group) having 1 to 20 carbon atoms; and
Q¹ is a divalent isocyanate residue.

Embodiment 12 is the composition of embodiment 11 wherein the one or more compounds of Component (A) are derived from the polymerization of at least one monomer of Formula (I).

Embodiment 13 is the composition of embodiment 12 wherein D¹ is —NH—, and the one or more polymeric compounds comprise a homopolymer of Formula (I).

Embodiment 14 is the composition of embodiment 12 or 13 wherein, in Component (A), each L¹, L², L³, L⁴, L⁵, L⁶, L⁷, L⁸, L⁹, and L¹⁰ is independently a straight chain (i.e., linear) alkylene group having 2 to 10 carbon atoms (in certain embodiments, an ethylene group, a butylene group, or a propylene group).

Embodiment 15 is the composition of any one of embodiments 1 through 14 wherein, in Component (A), the one or more polymeric compounds are derived from the polymerization of at least one monomer of Formula (I) and at least one monomer of Formula (II).

Embodiment 16 is the composition of any one of embodiments 11 through 15 wherein, in Component (A), the one or more polymeric compounds additionally include units derived from monomer with one or more functional groups capable of undergoing additional reactions.

Embodiment 17 is the composition of embodiment 16 wherein the functional groups capable of undergoing additional reactions are selected from a polymerizable olefin group, an olefin group that can undergo a hydrosilation reaction, an epoxy group, a hydroxyl group, a halo group, a haloformyl group, an aziridino group, an acid group, a salt of an acid group, an amino group, a salt of an amino group, a quaternary ammonium group, a salt of a quaternary ammonium group, a blocked isocyanate group, a hydroxyalkyl group, a chlorinated hydroxyalkyl group, an N-methylol group, an acetoacetoxyalkyl group, and a combination thereof.

Embodiment 18 is the composition of any one of embodiments 11 through 17 wherein the one or more polymeric compounds are derived from at least one monomer of the following formulas:

C₁₈H₃₇—NH—C(O)C(R²)═CH₂;

C₁₈H₃₇ —NH—C(O)OCH₂CH₂OC(O)C(R²)═CH₂;

C₁₈H₃₇—NH—C(O)—NH—C(O)—C(R²)═CH₂; and

C₁₇H₃₅—C(O)OCH₂CH₂C(O)C(R²)═CH₂.

wherein R² represents H or CH₃.

Embodiment 19 is the composition of embodiment 18 wherein the one or more polymeric compounds are derived from at least one monomer of the following formula:

C₁₈H₃₇—NH—C(O)C(R²)═CH₂

wherein R² represents H or CH₃ (preferably a homopolymer of C₁₈H₃₇—NH—C(O)C(H)═CH₂).

Embodiment 20 is the composition of any one of embodiments 6 through 19 wherein, in Component (A), the one or more polymeric compounds have a weight average molecular weight of greater than 20,000 Daltons and up to 500,000 Daltons.

Embodiment 21 is the composition of any one of embodiments 6 through 20 wherein, in Component (A), the one or more polymeric compounds have an average of greater than 20 repeating units of at least one monomer (preferably, at least one monomer of Formula (I)).

Embodiment 22 is the composition of any one of embodiments 6 through 21 wherein the components for making the one or more compounds of Component (B) comprise at least one additional mono-, di-, or poly-functional isocyanate reactive compound.

Embodiment 23 is the composition of embodiment 22 wherein the additional mono-, di-, or poly-functional isocyanate-reactive compound comprises a compound comprising: a hydrocarbon group having 4 to 60 carbon atoms (16 to 60 carbon atoms, 16 to 50 carbon atoms, 16 to 40 carbon atoms, or 16 to 30 carbon atoms); a polydimethylsiloxane segment having a weight average molecular weight of at least 200; a divalent polyoxyalkylene group comprising 2 to 100 alkylene oxide repeating units, wherein the alkyleneoxide unit has 2 to 10 carbon atoms: or a combination thereof.

Embodiment 24 is the composition of embodiment 23 wherein the additional mono-, di-, or poly-functional isocyanate-reactive compound comprises a mono-, di-, or poly-functional alcohol, thiol, amine, acid, or amide.

Embodiment 25 is the composition of any one of embodiments 6 through 24 wherein the components for making the one or more compounds of Component (B) comprises a blocked isocyanate group.

Embodiment 26 is the composition of embodiment 25 wherein the blocked isocyanate group is an oxime-derived group.

Embodiment 27 is the composition of any one of embodiments 6 through 26 wherein the isocyanate-reactive oligomer for making the one or more compounds of Component (B) are made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group (in certain embodiments, a linear or branched hydrocarbon group, and in certain embodiments, a linear hydrocarbon group) having from 4 to 60 carbon atoms (16 to 60 carbon atoms, 16 to 50 carbon atoms, 16 to 40 carbon atoms, or 16 to 30 carbon atoms), and at least one isocyanate-derived group.

Embodiment 28 is the composition of any one of embodiments 6 through 27 wherein, in Component (B), less than 40% of the isocyanate groups are reacted with acid and/or amide groups.

Embodiment 29 is the composition of any one of embodiment 28 wherein the isocyanate-reactive oligomer has the following formula:

Y¹[CH₂—C(R²)C(O)-D¹-R¹]ₘ—S—R¹⁰-(T¹)ₚ Formula (V)
wherein:
   Y¹ is H or an initiator residue;
   R¹ is a hydrocarbon group having 4 to 60 carbon atoms (16 to 60 carbon atoms, 16 to 50 carbon atoms, 16 to 40 carbon atoms, or 16 to 30 carbon atoms);
   R² is independently H or CH₃;
   D¹ is selected from:
      —C(O)O-L¹-O—;
      —OC(O)-L²-O—;
      —OC(O)-L³ -C(O)O-L⁴-O—;
      —C(O)NH-L⁵-O—;
      NHC(O)-L⁶-O—;
      —NHC(O)-L⁷-C(O)O-L⁸-O—;
      —C(O)—NH—;
      —C(O)NHC(O)NH-L⁹-O—;
      —C(O)NHC(O)-L¹⁰-O—;
      —NHC(O)NH—; and
      —NH—; with the proviso that when D¹ is —NH—, the isocyanate-reactive oligomer comprises greater than 30 wt-% of monomeric units that include —NH—, based on the total weight of monomeric units;
   each L¹, L², L³, L⁴, L⁵, L⁶, L⁷, L⁸, L⁹, and L¹⁰ is independently a branched or straight chain alkylene group having 2 to 10 carbon atoms;
   S is sulfur;
   R¹⁰ is a divalent or trivalent linking group having 1 to 10 carbon atoms;
   T¹ is —C(O)OH, —C(O)NH₂, —OH, —NH₂, or —NH(R¹¹), wherein R¹¹ is 1 or a hydrocarbon group having 1 to 10 carbon atoms;
   m is an integer of 2 to 20; and
   p is independently 1 or 2.

Embodiment 30 is the composition of embodiment 29 wherein, in Component (B), D¹ is —N—, and the oligomer includes 100% of monomeric units that include —NH—.

Embodiment 31 is the composition of embodiment 29 or 30 wherein, in Component (B), each L¹, L², L³, L⁴, L⁵, L⁶, L⁷, L⁸, L⁹, and L¹⁰ is independently a straight chain (i.e., linear) alkylene group having 2 to 10 carbon atoms.

Embodiment 32 is the composition of any one of embodiments 29 through 31 wherein, in Component (B), each L¹¹, L¹², and L¹³ is independently a straight chain (i.e., linear) alkylene group having 2 to 10 carbon atoms.

Embodiment 33 is the composition of any one of embodiments 6 through 32 wherein, in Component (B), the at least one polyisocyanate comprises an aromatic diisocyanate, an aromatic triisocyanate, an aromatic polymeric isocyanate, or a mixture thereof.

Embodiment 34 is the composition of any one of embodiments 1 through 33 which is an aqueous dispersion optionally comprising one or more additives selected from a paraffin wax, a surfactant, a coalescing solvent, an anti-freeze solvent, an emulsifier, and a stabilizer against one or more microorganisms.

Embodiment 35 is the composition of any one of embodiments 1 through 34 which is a treating composition.

Embodiment 36 is the composition of embodiment 35 which is a fluorine-free treating composition.

Embodiment 37 is a method of treating a fibrous substrate, the method comprising applying a treating composition of embodiment 35 or 36 in an amount sufficient to make the fibrous substrate water repellent.

Embodiment 38 is the method of embodiment 37 comprising applying the treating composition in an amount sufficient to make the fibrous substrate durably water repellent.

Embodiment 39 is a fibrous substrate treated by the method of embodiment 37 or 38.

Embodiment 40 is the fibrous substrate of embodiment 39 which is selected from the group of textile, leather, carpet, paper, and fabrics.

### EXAMPLES

Objects and advantages of this invention are further illustrated by the following examples, but the particular materials and amounts thereof recited in these examples, as well as other conditions and details, should not be construed to unduly limit this invention. These examples are merely for illustrative purposes only and are not meant to be limiting on the scope of the appended claims.

MATERIALS LIST

| Material | Description | Source |
|---|---|---|
| HOEA | 2-Hydroxyethyl acrylate, | Sigma-Aldrich, |
| | monomer | Belgium |
| HOBA | 4-Hydroxybutyl acrylate, | Sigma-Aldrich, |
| | monomer | Belgium |
| ODA | Octadecyl acrylate, | Sigma-Aldrich, |
| | monomer | Belgium |
| BAc, behenic acid | Docosanoic acid, | Oleon, Belgium |
| | available as | |
| | Radiacid 0560 | |
| Unicid ™ 350 | Fully saturated, | Baker Hughes, France |
| | long chain linear | |
| | acid with acid | |
| | number of 120 | |
| Unilin ™ 350 | Fully saturated, | Baker Hughes, France |
| | long chain linear | |
| | primary alcohol with | |
| | hydroxyl number of 137 | |
| SAnh | Succinic anhydride | Sigma-Aldrich, |
| | | Belgium |
| SAm, stearyl amine | Octadecyl amine | Sigma-Aldrich, |
| | | Belgium |
| AM | Acrylamide, | Sigma-Aldrich, |
| | monomer | Belgium |
| ACl | Acryloylchloride | Sigma-Aldrich, |
| | | Belgium |
| SCl, stearoyl chloride | Octadecanoyl chloride | Sigma-Adrich, |
| | | Belgium. |
| 2-mercaptoethanol | HSCH₂CH₂OH, | Sigma-Aldrich, |
| | chain transfer agent | Belgium |
| 16- | Chain transfer agent | Sigma-Aldrich, |
| mercaptohexadecanoic | | Belgium |
| acid | | |
| 3-mercaptopropionic | Chain transfer Agent | Sigma-Aldrich, |
| acid | | Belgium |
| SI (stearyl isocyanate) | Octadecyl isocyanate | Sigma-Aldrich, |
| | | Belgium |
| V-50 | 2,2′-Azobis(2- | Commercially |
| | methylpropionamidine) | available under the |
| | dihydrochloride | trade designation |
| | | “VAZO V-50” from |
| | | Wako Pure Chemical |
| | | Industries, Ltd., |
| | | Germany |
| V-59 | 2,2′-Azobis(2-methylbutyronitrile), | Commercially |
| | initiator | available under the |
| | | trade designation |
| | | “VAZO V-59” from |
| | | Wako Pure Chemical |
| | | Industries, Ltd., |
| | | Germany |
| VAZO-67 | Azonitrile free | Commercially |
| | radical initiator | available under the |
| | | trade name “VAZO- |
| | | 67” from DuPont, |
| | | Wilmington, DE |
| Ethylacetate | ethylacetate, | Sigma-Aldrich, |
| | solvent | Belgium |
| SA, stearylalcohol | Octadecyl alcohol, | Sigma-Aldrich, |
| | reactant | Belgium |
| isoSA, | isostearyl alcohol | Sigma-Aldrich, |
| | | Belgium |
| SAc, stearic acid | Octadecanoic acid | Sigma-Aldrich, |
| | | Belgium |
| DBTDL | Dibutyltin dilaurate, | Sigma-Aldrich, |
| | catalyst | Belgium |
| Zirconium | catalyst | Sigma-Aldrich, |
| isopropoxide | | Belgium |
| MPPO | 3-methyl-1-phenyl-2-phospholene | Sigma-Aldrich, |
| | 1-oxide, 85% technical grade, | Belgium |
| | carbodiimidization catalyst | |
| SAmd | Octadecanamide, | Sigma-Aldrich, |
| | stearylamide | Belgium |
| AOI | isocyanatoethylacrylate | Showa Denko, Japan |
| MSA | Methanesulphonic | Sigma-Aldrich, |
| | acid | Belgium |
| MEHQ | 4-methoxyphenol | Sigma-Aldrich, |
| | | Belgium |
| MEKO | 2-butanoneoxime, | Sigma-Aldrich, |
| | blocking agent | Belgium |
| ARMOCARE VGH- | Ester based quaternary, | Commercially |
| 70 | emulsfier | available under the |
| | | trade designation |
| | | “ARMOCARE VGH-70” |
| | | from Akzo Nobel, |
| | | Stenungsund, Sweden |
| PAPI | Polymethylenepolyphenylisocyanate | Covestro, Germany |
| Ethoquad C-12 | Quatemized coco | Commercially |
| | amine ethoxylate, | available under the |
| | emulsifier | trade designation |
| | | “Ethoquad C-12” |
| | | from Akzo Nobel, |
| | | Stenungsund, Sweden |
| TERGITOL TMN-6 | Nonionic emulsifier | Dow Chemical, |
| | | Midland, MI |
| TERGITOL 15-S-30 | Nonionic emulsifier | Dow Chemical, |
| | | Midland, MI |
| PES | Polyester Microfiber | Chyang Sheng Dyeing |
| | | and Finishing |
| | | Company Ltd., Taiwan |
| PA | Polyamide Microfiber, | Sofinal NV, Belgium |
| | style 6145 | |
| AA | Acrylic acid | Sigma-Aldrich, |
| | | Belgium |
| MA | Methacrylic acid | Sigma-Aldrich, |
| | | Belgium |
| CEA | 2-carboxyethylacrylate | Sigma-Aldrich, |
| | | Belgium |
| MAM | methacrylamide | Sigma-Aldrich |

### Test Method

### Spray Rating (SR)

The spray rating of a treated fibrous substrate is a value indicative of the dynamic repellency of the treated substrate to water that impinges on the treated substrate. The spray rating was measured by Test Method 22-2005, published in the 2001 Technical Manual of the American Association of Textile Chemists and Colorists (AATCC). The spray rating was obtained by spraying 250 milliliters (mL) water on the substrate from a height of 15 centimeters (cm). The wetting pattern is visually rated using a scale from 0 to 100, where 0 means complete wetting and 100 means no vetting at all. The spray rating was measured initially (recorded as SR IN) and after the fibrous substrate was laundered 10 or 20 times.

The laundering procedure consisted of placing a 400-900 cm² sheet of treated fibrous substrate in a washing machine (Miele Novotronic T490) along with ballast sample (1.9 kilogram (kg) of 8-ounce fabric). A commercial detergent (“Sapton,” available from Henkel, Germin, 60 grams (g)) was added. The fibrous substrate and ballast load were washed using a short wash cycle at 40° C., followed by a rinse cycle and centrifuging. The sample was not dried between repeat cycles. After the 10 or 20 washing cycles, the samples were dried in a Miele T-356 tumble drier, setting ‘Extra dry’. The samples were conditioned overnight at room temperature before the spray rating was tested The results of spray rating are indicated as SR 10L and SR 20L respectively.

As indicated in the Examples, the dried test samples were optionally ironed at 180° C. during 3 seconds. The samples were conditioned overnight at room temperature before the spray rating was tested. The results of spray rating after ironing, are indicated as SR 10L JR and SR 20L IR respectively.

### Treatment Procedure Via Padding Process

Before application to the fibrous substrates (e.g., textile or fabric substrates), the water based dispersions were diluted with DI-water to obtain treatment dispersions that provide 0.6% or 1% solids on fibrous substrate (SOF), as indicated in the examples. The treatments were applied onto the fibrous substrates, by immersing the substrates in the treatment dispersion and agitating until the substrate was saturated. The saturated fibrous substrate was then run through a padder/roller to remove excess of the dispersion and to obtain a certain Percent (%) Wet Pick Up (WPU) (100% WPU means that after this process the substrate absorbed 100% of its own weight of the treatment dispersion before drying). After application of the treatment dispersion, the treated substrates were dried and cured for 2 minutes at 180° C. unless otherwise indicated. The dried samples were conditioned overnight at room temperature before testing.

### Examples

### Preparation of (Meth)Acrylate and (Meth)Acrylamide Monomers

### Esteralkyl-Containing (Meth)Acrylate Monomers

HOBA/SAc Made from 4-Hydroxybutyl Acrylate and Stearic Acid

In a three-necked flask of 500 ml, fitted with a Dean Stark trap and condenser, thermometer, stirrer and heating mantle, were placed 43.2 g (0.3 mol) HOBA, 85.2 g (0.3 mol) SAc, 60 g toluene, 0.2 g MSA, 0.06 g MEI-Q and 0.06 g PTZ (phenothiazine). An azeotropic distillation was started for 8 hours, after which time, about 5.4 g water was collected. NMR analysis indicated that the conversions of HOBA and SAc were about 99% and no homopolymer was formed. The structure of the acrylate monomer, referred to as HOBA/SAc was C₁7H₃₅C(O)O(CH₂)₄OC(O)CH═CH₂, representing an esteralkyl-containing acrylate, which is Formula (I)

wherein R¹ is C₁₇H₃₅—, R² is H and D¹ is —C(O)O-L¹-O—, wherein V is —(CH₂)₄—.

Using the same procedure, monomers HO-BA/BAc, (C₂₁H₄₃C(O)O(CH₂)₄OC(O)CH═CH₂), made from 4-hydroxybutyl acrylate and behenic acid; (Formula (I), wherein R¹ is C₂₁H₄₃—, R² is H and D¹ is —C(O)O-L¹-O—, wherein L¹ is —(CH₂)₄—); HOBA/UNICID 350, CₙH₂ₙ₊₁C(O)O(CH₂)₄OC(O)CH═CH₂, made from 4-hydroxybutyl acrylate and UNICID 350 (Formula (I), wherein R is CₙH₂ₙ₊₁—, n=about 30; R² is Hand D¹ is —C(O)O-L¹-O—, wherein L is —(CH₂)—; HOEA/UNICID 350, (CₙH₂ₙ₊₁C(O)O(CH₂)₂OC(O)CH═CH₂), made from hydroxyethyl acrylate and UNICID 350 (Formula (I), wherein R¹ is CₙH₂ₙ₊₁—, n=about 30; R² is H and D¹ is —C(O)O-L¹-O—, wherein L¹ is —(CH₂)— were prepared.

Using the same procedure, monomer UNILIN 350/SAnh/HOBA, CₙH₂ₙ₊₁OC(O)(CH₂)₂C(O)O(CH₂)₄OC(O)CH═CH₂, was made from UNILIN 350, succinic anhydride and 4-hydroxybutyl acrylate. This monomer is Formula (I) wherein R¹ is CₙH₂ₙ₊₁—, n=about 30; R² is H and D¹ is —OC(O)-L³-C(O)O-L⁴-O—, wherein L³ is —(CH₂)₂— and L⁴ is —(CH₂)₄—.

### (Meth)Acrylamide Monomers

SI-AA Made from Stearyl Isocyanate (SI) and Acrylic Acid

In a three-necked flask of 500 ml fitted with a stirrer, heating mantle, addition funnel, condenser and thermometer were placed 17.3 g (0.24 mol) of AA, 70 g THF (tetrahydrofuran, available form Sigma-Aldrich), 0.05 g MgCl₂ (available from Sigma-Aldrich), 0.04 g MEHQ and 0.04 g PTZ under nitrogen atmosphere. Through the addition funnel 70.8 g (0.24 mol) SI were added dropwise over about 1 hour. Gas evolution was noticed. In order to keep the reaction mixture in solution, the temperature was gradually raised to 70° C. After addition of SI was completed, the reaction was continued for another 3 hours until all outgassing had stopped. A clear solution was obtained in THF at 70° C. IR analysis indicated that all isocyanate had reacted. NMR-analysis indicated that about 92% C₁₈H₃₇NH—C(O)CH═CH₂, stearylacrylamide, was formed.

Using the same procedure, monomer ST-MA was made from stearyl isocyanate and methacrylic acid, leading to C₁₈H₃₇NH—C(O)C(CH₃)═CH₂, stearyl methacrylamide.

SI-AA and SI-MA are according to Formula (I) wherein R¹ is C₁₈H₃₇, D¹ is —NH— and R² is —H or —CH₃ respectively.

ACl-SAm Made from Acryloylchloride and Stearyl Amine

In a three-necked flask of 1000 ml fitted with a stirrer, heating mantle, addition funnel, condenser and thermometer, were placed 80.7 g SAm (0.3 mol), 0.045 g MEHQ, 0.045 PTZ, 33.3 g (033 mol) dry triethylamine (Sigma Aldrich-Belgium) and 500 g dry 2-methyltetrahydrofuran (Aldrich-Belgium) under a nitrogen atmosphere in an ice bath at about 5° C. Then 30.3 g ACl (0.33 mol) were added dropwise to keep the temperature below 20° C. A lot of white precipitate was formed. The temperature was raised to 70° C. for 3 hours. The reaction mixture was washed 3 times with 100 g water at about 70° C. and the organic layer was collected. Solvent was evaporated using aspirator vacuum. GLC analysis indicated formation of C₁₈H₃₇—NHC(O)CH═CH₂, stearyl acrylamide, in about 92% yield. ACl-SAm is according to Formula (I) wherein R¹ is C₁₈H₃₇—, D¹ is —NH— and R² is H.

### Acyl Containing (Meth)Acrylamide Monomers

SCI-AM Made from Stearoyl Chloride (SCI) and Acrylamide (AM)

In a three-necked flask of 500 ml fitted with a stirrer, heating mantle, condenser and thermometer were placed 73.8 g (0.244 mol) SCI and 40 g MEK (2-butanone) under a nitrogen atmosphere. A solution of 14.2 g AM (0.2 mol), 24.9 g (0.244 mol) triethylamine, 0.045 g MEHQ, 0.045 g PTZ and 160 g MEK was prepared in a separate glass flask of 250 ml and slowly added to the SC/MEK solution over about 1 hour. The temperature of the reaction mixture was gradually increased from 25° C. to 80° C. to keep the formed viscous slurry liquid. The reaction was continued for 16 hours at 80° C. A viscous slurry containing the acyl amide (imide) monomer, MEK and the triethylammonium chloride was obtained. The mixture was washed 3 times with 100 g of water at 80C and the organic layer was collected. Solvent was evaporated to yield C₁₇H₃₅—C(O)—NH—C(O)CH═CH₂ (N-octadecanoyl acrylamide).

Using the same procedure, SCl-MAM (N-octadecanoyl methacrylamide) was prepared from stearoyl chloride (SCI) and methacrylamide (MAM)

SCI-AM and SCI-MAM are Formula (T) wherein R¹ is C₁₇H₃₅—, D¹ is —C(O)NH— and R² is H or CH₃ respectively.

### Amide Containing (Meth)Acrylate Monomers

AOI-SAc Made from Isocyanato Ethyl Acrylate and Stearic Acid

In a three-necked flask of 500 ml, equipped with a stirrer, heating mantle, addition funnel, condenser and thermometer, were placed 85.2 g SAc (0.3 mol), 80 g dry 2-methyltetrahydrofuran, 0.05 g MgCl₂, 0.045 g MEHQ and 0.045 g PTZ under nitrogen atmosphere. Through the addition funnel 42.3 g AOI (0.3 mol) were added dropwise over about 1 hour. Immediate gas evolution was noticed. During addition, the temperature was gradually increased to 70° C. After all AOI was added, the reaction was continued at 70° C. for another 3 hours until gas evolution had stopped. IR analysis indicated that all isocyanate had disappeared. NMR analysis indicated formation of C₁₇H₃₅C(O)NHCH₂CH₂OC(O)CH═C₂ in about 87% yield.

AOI-SAc is Formula (I), wherein R¹ is C₁₇H₃₅—, R² is H, D¹ is —C(O)NH-L⁵-O—, wherein L⁵ is —CH₂CH₂—.

Using the same procedure SI-CEA was prepared from stearyl isocyanate (SI) and 2-carboxyethylacrylate (CEA).

SI-CEA is Formula (I), wherein R¹ is C₁₈H₃₇—, R² is H and D¹ is —NHC(O)-L⁶-O—, wherein L⁶ is —CH₂CH₂—.

### Amido- and Ester-Containing (Meth)Acrylate Monomers

Sam-SAnh-HOBA Made from Stearylamine, Succinic Anhydride and 4-Hydroxybutyl Acrylate

In a three-necked flask of 500 ml, fitted with a stirrer, condenser, thermometer and heating mantle, were placed 80.8 g (0.3 mol) SAm and 30 g (0.3 mol) SAnh. The reaction mixture was heated up to 90° C. under nitrogen atmosphere. An exothermic effect was noticed and the reaction was continued for 2 hours at 90° C. Then, 70 g toluene, 43.2 g (0.3 mol) HOBA, 0.2 g MSA, 0.06 g MEHQ and 0.06 g PTZ were added and a Dean Stark azeotropic distillation unit was set up. An azeotropic distillation was started for 8 hours, after which about 5.3 g water was collected. NMR indicated formation of monomer containing two ester groups and one amide group. No homopolymer was detected.

The monomer formed, i.e. C₁₈H₃₇NHC(O)(CH₂)₂C(O)O(CH₂)₄OC(O)CH═CH₂, is Formula (I) wherein R¹ is C₁₈H₃₇—, R² is H, D¹ is —NHC(O)-L⁷-C(O)O-L⁸-O—, wherein L⁷ is —(CH₂)₂— and L⁸ is —(CH₂)₄—.

### Urea-Containing (Meth)Acrylate Monomers

SI-AM Made from Stearyl Isocyanate (SI) and Acrylamide (AM)

In a three-necked flask of 500 ml fitted with a stirrer, heating mantle, addition funnel, condenser and thermometer were placed 35 g (0.5 mol) of AM, 177 g (0.6 mol) ST, 0.1 g MEHQ, 0.1 g PTZ and 60 g butylacetate, under nitrogen atmosphere. At 50° C. the mixture became clear. Heating up to 120° C. continued for 4 hours. A clear, slightly pink solution was obtained. IR analysis indicated that all isocyanate had reacted. NMR-analysis indicated formation of about 84% C₁₈H₃₇NH—C(O)—NH—C(O)CH═CH₂ (1-octadecyl 3-acryloyl urea).

Using the same procedure, S-MAM, C₁₈H₃₇NH—C(O)—NH—C(O)C(CH₃)═CH₂, was prepared from stearyl isocyanate (SI) and methacrylamide (MAM).

SI-AM and SI-MAM are Formula (I), wherein R¹ is C₁₈H₃₇—, D¹ is —NH—C(O)—NH— and R² is H.

### Acylurea-Containing (Meth)Acrylate Monomers

AOI-SAmd Made from Isocyanato Ethyl Acrylate and Stearyl Amide

In a three-necked flask of 500 ml fitted with a stirrer, heating mantle, condenser and thermometer, were placed 42.3 g AOI (0.3 mol), 84.9 g SAmd (0.3 mol), 0.1 g MEHQ, 0.1 g PTZ and 50 g BuAc (butyl acetate) under a nitrogen atmosphere. The reaction mixture was heated up to 120C for 4 hours. A clear slightly pink yellow solution was obtained. JR analysis indicated that all isocyanate groups had reacted. NMR analysis indicated formation of C₁₇H₃₅C(O)NHC(O)NHCH₂CH₂OC(O)CH═CH₂ (1-ethylacrylol, 3-octadecanoyl urea) with about 82% yield.

AOI-SAmd is Formula (I), wherein R¹ is C₁₇H₃₅—, R² is H, D¹ is —C(O)NHC(O)NH-L⁹-O—, wherein L⁹ is —CH₂CH₂—.

### Urethane-Containing (Meth)Acrylate Monomers

ST-HOEA Made from Stearyl Isocyanate and 2-Hydroxyethyl Acrylate

In a three-necked flask of 500 ml fitted with a stirrer, heating mantle, condenser and thermometer were placed 70.8 g SI (024 mol), 27.8 g HOEA (024 mol), 35 g ethylacetate, 0.05 g MEHQ, 0.05 g PTZ and 1 drop of DBTDL. The reaction mixture was heated up to 80C for 5 hours. A clear solution of SI-HOEA monomer resulted. IR indicated that all isocyanate had reacted. NMR-analysis indicated about 94% yield of C₁₈H₃₇NHC(O)OCH₂CH₂OC(O)CH═CH₂. At room temperature, a hard solid, waxy material was obtained, referenced as “SI-HOEA.”

ST-HOEA is Formula (IT) wherein R³ is C₁₈H₃₇—, R⁴ is H, D² is —N—C(O)O-L¹⁰-O—, wherein L¹⁰ is —CH₂CH₂—.

### Preparation of Isocyanate-Reactive Oligomers

Isocyanate-Reactive Oligomers Derived from Ester-Containing (Meth)Acrylate Monomers

### (HOBA/SAc)₁₀—OH

In a three-necked flask of 500 ml, fitted with a Dean Stark trap and condenser, thermometer, stirrer and heating mantle, were placed 43.2 g (0.3 mol) HOBA, 85.2 g (0.3 mol) SAc, 60 g toluene, 0.2 g MSA, 0.06 g MEHQ and 0.06 g PTZ. An azeotropic distillation was started for 8 hours, after which about 5.4 g water was collected.

The reaction mixture was cooled to 70° C. under a nitrogen atmosphere and 2.3 g (0.03 mol) 2-mercaptoethanol and 0.23 g V-59 initiator were added. The mixture was warmed up to about 80° C. under nitrogen. The reaction mixture increased to about 85° C. The reaction was continued for 3 hours at 85° C. Another portion of 0.1 g V-59 was added and the reaction continued for another 5 hours. Finally, a third portion of 0.05 g V-59 was added and the reaction continued for 16 hours. A clear viscous solution resulted. NMR indicated formation of a hydroxy-functionalized oligomer. The toluene solvent was stripped off at about 80-90C with aspirator vacuum.

Using essentially the same procedure, (HOBA/BAc)₁₀-OH, (HOBA/UNICID 350)₄—OH, (HOEA/UNICID 350)₄—OH and (UNILIN 350/SAnh/HOBA)₄-OH were prepared by using the appropriate amounts of 2-mercaptoethanol.

(HOBA/SAc)₁₀-OH is an oligomer according to Formula (V), wherein Y¹ is H or an initiator residue, R¹ is C₁₇H₃₅—, R² is H, S is sulfur, R¹⁰ is —CH₂CH₂—, p is 1, in is 10, T¹ is —OH and D¹ is —C(O)O-L¹-O—, wherein L¹ is —(CH₂)⁴—.

(HOBA/BAc)₁₀—OH is an oligomer according to Formula (V), wherein Y¹ is H or an initiator residue, R¹ is C₂₁H₄₃—, R² is H, S is sulfur R¹⁰ is —CH₂CH₂—, p is 1, m is 10, T¹ is —OH, and D¹ is —C(O)O-L¹-O—, wherein L¹ is —(CH₂)₄—.

(HOBA/UNICID 350)₄—OH is an oligomer according to Formula (V), wherein Y¹ is H or an initiator residue, R¹ is CₙH₂ₙ₊₁—, n=about 30; R² is H, S is sulfur, R¹⁰ is —CH₂CH₂—, p is 1, is 4, T¹ is —OH— and D¹ is —C(O)O-L¹-O—, wherein L¹ is —(CH₂)₄—.

(HOEA/UNICID 350)₄—OH is an oligomer according to Formula (V), wherein Y¹ is H or an initiator residue, R¹ is CₙH₂ₙ₊₁—, wherein n=about 30; R² is H, S is sulfur, R¹⁰ is —CH₂CH₂—, p is 1, m is 4, T¹ is —OH and D¹ is —C(O)O-L¹-O—, wherein L¹ is —(CH₂)₂—.

(UNILIN 350/SAnh/HOBA)₄-OH is an oligomer according to Formula (V), wherein Y¹ is H or an initiator residue, R¹ is CₙH₂ₙ₊₁—, wherein n=about 30, R² is H, S is sulfur, R¹⁰ is —CH₂CH₂—, p is 1, m is 4, T¹ is —OH and D¹ is —OC(O)-L³-C(O)O-L⁴-O—, wherein L³ is —(CH₂)₂— and L⁴ is —(CH₂).

Isocyanate-Reactive Oligomers Derived from (Meth)Acrylamide Monomers (SI-AA)₈-OH

In a three-necked flask of 250 ml fitted with stirrer, condenser, thermometer and heating mantle, were placed 64.6 g (0.2 mol) of SI-AA monomer (as prepared above), 2 g (0.025 mol) HSCH2CH2OH, 35 g EtOAc and 0.1 g VAZO-67. Under nitrogen, the reaction mixture was warmed up to about 75° C. An exothermic effect was noticed, heating up the mixture to about 85° C. (vigorous reflux). The reaction was continued for 3 hours; 0.05 g VAZO-67 was added and the reaction continued for 16 hours at about 85° C. A viscous solution was obtained.

Using essentially the same procedure, oligomers (SI-AA)₆-OH—, (SI-AA)₁₀-OH, (SI-MA)₈-OH and (SI-MA)₁₀-OH were prepared by adjusting the amount of 2-mercaptoethanol accordingly.

Using essentially the same procedure (ACl-SAm)₈—OH was prepared but starting from ACI-SAm instead of SI-AA.

(SI-AA)₆-OH, (ST-AA)₈-OH, (ST-AA)₁₀-OH, (ST-MA)₈-OH, (ST-MA)₁₀-OH and (ACI-SAm)₈—OH are Examples of hydroxy functionalized isocyanate-reactive oligomers according to Formula (V), wherein Y¹ is H or an initiator residue, R¹ is C₁₈H₃₇, R² is H or CH₃, S is sulfur, R¹⁰ is —CH₂CH₂—, p is 1, m is 6, 8 or 10, T¹ is —OH and D¹ is —NH—.

Using essentially the same procedure (AOI-SAc)₈—OH was prepared but starting from AOI-SAc monomer instead of SI-AA.

(AOI-SAc)₈—OH is a hydroxy functionalized isocyanate-reactive oligomer according to Formula (V), wherein Y¹ is H or an initiator residue, R¹ is C₁₇H₃₅, R² is H, S is sulfur, R¹⁰ is —CH₂CH₂—, p is 1, m is 8, T¹ is —OH and D¹ is —C(O)NH-L⁵-O—, wherein L is-C₂CH₂—.

### (SI-AA)₈-COOH

In a three-necked flask of 250 ml fitted with stirrer, condenser, thermometer and heating mantle, were placed 64.6 g (0.2 mol) of SI-AA monomer (as prepared above), 2 g (0.025 mol) 3-mercaptopropionic acid, 35 g EtOAc and 0.1 g VAZO-67. Under nitrogen, the reaction mixture was warmed up to about 75° C. An exothermic effect was noticed, heating up the mixture to about 85° C. (vigorous reflux). The reaction was continued for 3 hours; 0.05 g VAZO-67 was added and the reaction continued for 16 hours at about 85° C. (SI-AA)₈-COOH is an acid functionalized isocyanate-reactive oligomer, according to Formula (V) wherein Y¹ is H or an initiator residue, R¹ is C₁₈H₃, R² is H, S is sulfur, R¹⁰ is —CH₂CH₂—, p is 1, m is 8, T¹ is —COOH and D¹ is —NH—.

### (SCI-AM)₆-OH

In a three-necked flask of 250 ml fitted with a stirrer, condenser, thermometer and heating mantle, were placed 67.6 g (0.2 mol) of SCI-AM monomer (as prepared above), 2.6 g (0.033 mol), 2-mercaptoethanol, 35 g ethylacetate and 0.1 g V-59 initiator. Under a nitrogen atmosphere, the reaction mixture was warmed up to about 75° C. An exotherm was noticed and the mixture was further heated to about 85° C. The reaction was continued for 3 hours. Then 0.05 g V-59 was added and the reaction continued for 16 hours at about 85° C. A viscous solution of the oligomer referred to as (SC-AM)₆-OH was obtained.

Using the same procedure (SC-MAM)₆-OH was prepared but using monomer SCl-MAM instead of monomer SCI-AM.

(SCI-AM)₆-OH and (SCI-MAM)₆-OH are hydroxy functionalized isocyanate-reactive oligomers according to Formula (V), wherein Y¹ is H or an initiator residue, R¹ is C₁₇H₃₅, R² is H or CH₃, S is sulfur, R⁵ is —CH₂CH₂—, p is 1, m is 6, T¹ is —OH and D¹ is —C(O)—NH—.
Isocyanate Reactive Oligomers Derived from Acylurea- and Urea-Containing (Meth)Acrylate Monomers

### (AOI-SAmd)₈-OH

In a three-necked flask of 250 m fitted with a stirrer, heating mantle, thermometer and condenser were placed 84.8 g (0.2 mol) of AOI-SAmd monomer (as prepared above), 1.95 g (0.025 mol) 2-mercaptoethanol, 35 g ethylacetate and 0.1 g V-59 initiator. Under nitrogen atmosphere the reaction temperature was warmed up to about 70° C. An exothermic effect was noticed resulting in strong reflux of the reaction mixture. The reaction was continued for 3 hours at 85° C. Then 0.05 g of V-59 was added and the reaction was continued for 16 hours at about 85° C. A viscous solution was obtained. The obtained hydroxy functionalized isocyanate-reactive oligomer was referred to as (AOl-SAmd)₈-OH and is according to Formula (V) wherein Y¹ is H or an initiator residue, R¹ is C₁₇H₃₅—, R² is H, S is sulfur, R¹¹ is —CH₂CH₂—, p is 1, m is 8, T¹ is —OH and D¹ is —C(O)NHC(O)NH-L⁹-O—, wherein L⁹ is —CH₂CH₂—.

Using essentially the same procedure (SI-AM)₆-OH and (SI-MAM)₆-OH were prepared, but starting from the monomers SI-AM and SI-MAM respectively, instead of AOI-SAmd and using 0.033 mol of 2-mercaptoethanol per 0.2 mol monomer. (SI-AM)₆-OH and (SI-MAM)₆-OH are hydroxy functionalized isocyanate-reactive oligomers according to Formula (V), wherein i is H or an initiator residue, R¹ is C₁₈H₃₇, R² is H or CH₃ respectively, S is sulfur, R¹¹ is —CH₂CH₂—, p is 1, in is 6, T¹ is —OH and D¹ is —N—C(O)—NH—.

Using essentially the same procedure as for (AOI-SAmd)₈-OH, (SI-AM)₆-COOH was prepared, but using 3-mercaptopropionic acid instead of 2-mercaptoethanol.

(SI-AM)₆-COOH is an acid functionalized isocyanate-reactive oligomer according to Formula (V), wherein Y¹ is H or an initiator residue, R¹ is C₁₈H₃₇, R² is H, S is sulfur, R⁵ is —CH₂CH₂—, p is 1, in is 6, T¹ is —COOH and D¹ is —NH—C(O)—NH—.

Isocyanate Reactive Oligomers Derived from Urethane-Containing (Meth)Acrylates

### (SI-HOEA)₆-OH

In a three-necked flask of 250 ml fitted with a stirrer, condenser, thermometer and heating mantle, were placed 82.2 g (0.2 mol) of SI-HOEA monomer (as prepared above), 2.6 g (0.033 mol) 2-mercapto ethanol, 35 g ethylacetate and 0.1 g V-59 initiator. Under a nitrogen atmosphere, the reaction mixture was warned up to about 75° C. An exotherm was noticed and the mixture was further heated to about 85° C. The reaction was continued for 3 hours. Then 0.05 g V-59 was added and the reaction continued for 16 hours at about 85° C. A viscous solution comprising an oligomer, referred to as (SI-HOEA)₆-OH was obtained.

(SI-HOEA)₆-OH is an example of a hydroxy functionalized isocyanate-reactive oligomer, comprising 6 units of a urethane-containing acrylate monomer and is according to Formula (VT) wherein Y² is H or an initiator residue, R³ is C₁₈H₃₇, R⁴ is H, S is sulfur, R¹² is —CH₂CH₂—, p is 1, m is 6, T² is —OH and D² is —NHC(O)O-L¹¹-O—, wherein L¹¹ is-CH₂CH₂—.

Using essentially the same procedure, (SI-HOEA)₈-COOH was prepared, but using 26 g (0.025 mol) of 3-mercaptopropionic acid instead of 2-mercapto ethanol. (ST-HOEA)₈-COOH is an acid functionalized isocyanate-reactive oligomer according to Formula (VI), Wherein Y² is H or an initiator residue, R³ is C₁₈H₃₇—, R⁴ is H, S is sulfur, R¹² is —CH₂CH₂—, p is 1, in is 8, T² is —COOH and D² is —NHC(O)O-L¹-O—, wherein L¹¹ is —CH₂CH₂—.

Using essentially the same procedure, (SI-HOEA)₈-C₁₆ COOH was prepared but using 7.2 g (0.025 mol) of 16-mercaptohexadecanoic acid (MHA) instead of 2-mercapto ethanol.

(SI-HOEA)₈-C₁₆ COOH is an example of an acid functionalized isocyanate-reactive oligomer and according to Formula (VI), wherein Y² is H or an initiator residue, R³ is C₁₈H₃₇, R⁴ is H, S is sulfur, R¹² is —C₁₆H₃₂CH₂CH₂—, p is 1, m is 8, T2 is —COOH and D² is —NHC—C(O)O-L¹¹-O—, wherein L¹¹ is —CH₂CH₂—

### (ODA)₈-COOH

In a three-necked flask of 500 ml fitted with a stirrer, condenser, thermometer and heating mantle, were placed 64.8 g (0.2 mol) of ODA monomer, 2.65 g (0.025 mol) 3-mercaptopropionic acid, 35 g ethylacetate and 0.1 g V-59 initiator. Under a nitrogen atmosphere, the reaction mixture was warned up to about 75° C. An exotherm was noticed and the mixture was further heated to about 85° C. The reaction was continued for 3 hours. Then 0.05 g V-59 was added and the reaction continued for 16 hours at about 85° C. A viscous solution comprising an acid functionalized isocyanate-reactive oligomer, referred to as (ODA)₈-COOH, was obtained.

(ODA)₈-COOH is an example of an acid functionalized isocyanate reactive oligomer and is according to Formula (VI), wherein Y² is H or an initiator residue, R³ is C₁₈H₃₇ —, R⁴ is H, S is sulfur, R¹² is —CH₂CH₂—, p is 1, m is 8, T² is —COOH and D² is —O—

### Polymeric Compounds

### Poly(SI-HOEA)

Poly(SI-HOEA) (or p(SI-HOEA)) was prepared via mini-emulsion. Therefore, in a first step, a dispersion was made by dispersing 50 g SI-HOEA (synthesis as given above) in a mixture of 13 g Ethoquad C12, 3 g TERGITOL TMN-6 and 1.5 g TERGITOL 15-S-30, and 128 g D.I. water via ultrasone at a temperature of 95° C. This ‘monomer’ dispersion was then polymerized after the addition of 0.3 g V-50 initiator, degassing and reacting in a 95° C. preheated launder-o-meter for 6 hrs.

p(SI-HOEA) is a polymeric compound derived from a urethane-containing acrylate monomer according to Formula (II) wherein R³ is C₁₈H₃₇, R⁴ is H, D² is —NHC(O)O-L¹¹-O—, wherein L is —CH₂CH₂—.

### Example 1 (ST-AA)₁₀-OH/SA/PAPI/MEKO (0.6/0.6/3/1.8)

In a three-necked flask of 250 ml fitted with stirrer, condenser, thermometer and heating mantle, were placed 64.6 g (0.2 mol) of SI-AA acrylamide monomer, 1.6 g (0.02 mol) 2-mercaptoethanol, 35 g ethylacetate and 0.1 g VAZO-67. Under nitrogen atmosphere, the reaction mixture was warmed up to about 75° C. An exothermic effect was noticed heating up the mixture to about 85C (vigorous reflux). The reaction was continued for 3 hours. Then 0.05 g VAZO-67 was added and the reaction continued for 16 hours at about 85° C. A viscous solution was obtained. Then 60 g ethylacetate, 13.6 g (0.1 equivalents) PAPI and 5.4 g (0.02 mol) of SA were added, together with 0.035 g zirconium isopropoxide catalyst. The reaction was continued at 82° C. for 16 hours. In a last step, 5.2 g (0.06 mol) MEKO was added and reacted for 3 hours at 82° C. IR analysis indicated that all isocyanate groups had reacted. The material is referenced to as (SI-AA)₁₀-OH/SA/PAPI/MEKO (0.6/0.6/3/1.8) where the numbers in parentheses refer to the relative equivalents of the 4 reactants identified by the previously described acronyms.

### Example 2 (SI-MA)₁₀-OH/SA/PAPI/MEKO (0.6/0.6/3/1.8)

Using the same procedure (SI-MA)₁₀-OH/SA/PAPI/MEKO (0.6/0.6/3/1.8) was prepared, starting from SI-MA instead of SI-AA.

The materials of Example 1 (EX-1) and Example 2 (EX-2) are fluorine-free compounds derived from reaction, carried out in one or more steps, of components comprising
(i) a hydroxy functionalized isocyanate-reactive oligomer, comprising 10 repeating units,
(ii) a polymeric isocyanate, PAPI;
(iii) a monofunctional isocyanate-reactive compound, stearyl alcohol, SA; and
(iv) an isocyanate blocking agent, MEKO.

wherein the isocyanate-reactive oligomer has the formula (V), wherein Y¹ is H or an initiator residue, R¹ is C₁₈H₃₇—, R² is H or CH₃, S is sulfur, R¹⁰ is —CH₂CH₂—, p is 1, m is 10, T¹ is —OH— and D¹ is —NH—.

### Emulsification Procedure

In a 1000-mL three-necked flask, fitted with a stirrer, heating mantle, thermometer and cooler were placed 200 g of a 50% solids (in ethyl acetate) containing reaction mixture as prepared above. The mixture was heated up to 70° C. and mixed until a clear solution in ethyl acetate was obtained. In a 1000-mL beaker were placed 3 g TERGITOL 15-S-30, 6 g TERGITOL TMN-6, 3.7 g ARMOCARE VGH-70, and 400 g DI-water. This mixture was warmed up to about 70° C. and then added under vigorous stirring to the above-mentioned solution in the 1000 ml three-necked flask. A pre-emulsion was obtained at 70° C. This pre-emulsion was passed 3 times through a pre-heated 2-step Manton-Gaulin homogenizer at a pressure of 300 bar. Solvent was stripped off at temperature of about 45 to 50° C. and vacuum of about 20-30 mm 1 g. A stable dispersion at about 20% solids in water resulted.

### Spray Rating Test

The water-based dispersions of Example 1 and Example 2 were applied to PES and PA fabrics according to the general procedure “Treatment Procedure via Padding Process, Water-based Dispersions” as given above. After the 10 or 20 washing cycles the textile samples were dried in a Miele T-356 tumble drier, setting ‘Extra dry’. The samples were only ironed after 20 launderings.

The treated fabrics were tested for their dynamic water repellent properties according to the “Spray Rating (SR)” test outlined above. The properties were tested initially and after 10 or 20 launderings. The samples were conditioned overnight at room temperature before testing. The results of spray rating were indicated as SR IN, SR 10L and SR 20L. The results are recorded in Table 1.

TABLE 1
Spray Ratings (0.6% SOF); Water-based Dispersions
(PES: 100% WPU; PA: 93% WPU)

| | PES | | | | PA | | | |
|---|---|---|---|---|---|---|---|---|
| | SR | SR | SR | SR | SR | SR | SR | SR |
| Ex. | IN | 10 L | 20 L | 20 L IR | IN | 10 L | 20 L | 20 L IR |
| EX-1 | 100 | 80 | 80 | 80 | 100 | 100 | 90 | 90 |
| EX-2 | 100 | 90 | 90 | 90 | 100 | 90 | 90 | 90 |

These results clearly demonstrate that fluorine-free treating compositions comprising the reaction product of a hydroxy functionalized isocyanate-reactive oligomer (of a (meth)acrylamide monomer comprising a hydrocarbon group having 18 carbon atoms) with a polymeric isocyanate, a monofunctional isocyanate-reactive compound, and an isocyanate blocking agent provides treated textile substrates, not only with initial high water repellency, but surprisingly also good laundering resistance, after 10 or even after 20 laundering cycles. The results further indicate that it is not necessary to iron the treated samples after extended laundering cycles.

### Example 3 (SI-HOEA)₈-COOH/SA/PAPI/MEKO (0.6/0.6/3/1.8)

In a three-necked flask of 500 ml fitted with a stirrer, heating mantle, an addition funnel and thermometer were placed a mixture of (0.025 mol (SI-HOEA)₈-COOH in EtOAc (as prepared above), 70 g additional EtOAc and 0.07 g MgCl₂. The reaction mixture was heated up to 70° C. under a nitrogen atmosphere. Then a solution of 17 g (0.125 equiv.) PAPI in 20 g EtOAc was added dropwise. CO₂ evolution was observed. After all PAPI was added, the reaction was continued for about 3 hours at 80° C. until all gas evolution had stopped. Then 6.7 g (0.025 mol) SA was added together with 1 drop of DBTDL. The reaction was continued for 16 hours at 83° C. As a last step, 6.5 g (0.075 mol) MEKO was added dropwise and the reaction was continued for 2 hours until no more isocyanate peak could be detected by IR. A clear, amber brown solution was obtained.

The material of Example 3 (EX-3) is a fluorine-free treating compound derived from reaction, carried out in one or more steps, of components comprising:
(i) an acid functionalized isocyanate-reactive oligomer, comprising 8 repeating units,
(ii) a polymeric isocyanate, PAPI;
(iii) a monofunctional hydroxyl containing isocyanate-reactive compound, stearyl alcohol, SA; and
(iv) an isocyanate blocking agent, MEKO;

wherein the isocyanate-reactive oligomer has Formula (VI), wherein Y² is H or an initiator residue, R³ is C₁₈H₃₇—, R⁴ is H, S is sulfur, R¹² is —CH₂CH₂—, p is 1, in is 8, T² is —COOH and D² is —NHC(O)O-L¹¹-O—, wherein L¹¹ is —CH₂CH₂—; and

wherein about 25% of the isocyanate groups of the polymeric isocyanate are reacted with acid groups.

### Examples 4 to 7

Examples 4 to 7 were all made according to the same procedure as given for Example 3.

The material of Example 4 (EX-4) is a fluorine-free compound that can be represented by (SI-HOEA)₆-OH/BAc/PAPI/MEKO (0.6/0.6/3/1.8) and is derived from reaction, carried out in one or more steps, of component comprising:
(i) a hydroxy functionalized isocyanate-reactive oligomer, comprising 6 repeating units;
(ii) a polymeric isocyanate, PAPI;
(iii) a monofunctional acid containing isocyanate-reactive compound, behenic acid, BAc; and
(iv) an isocyanate blocking agent, MEKO,

wherein the isocyanate-reactive oligomer has the Formula (VI), wherein Y² is H or an initiator residue, R³ is C₁₈H₃₇—, R⁴ is H, S is sulfur, R¹² is —CH₂CH₂—, p is 1, m is 6, T² is —OH— and D² is —N—C(O)O-L¹¹-O— wherein LE is —C₂CH₂—, and

wherein about 25% of the isocyanate groups of the polymeric isocyanate are reacted with acid groups.

The material of Example 5 (EX-5) is a fluorine-free compound that can be represented by (SI-HOEA)₈-C₁₆COOH/SA/PAPI/MEKO (0.6/0.6/3/1.8) and is derived from a reaction mixture of:
(i) an acid functionalized isocyanate-reactive oligomer, having 8 repeating units;
(ii) a polymeric isocyanate, PAPI;
(iii) a monofunctional hydroxyl containing isocyanate-reactive compound, stearyl alcohol, SA; and
(iv) an isocyanate blocking agent, MEKO;

wherein the isocyanate-reactive oligomer has Formula (VI), wherein Y² is H or an initiator residue, R³ is C₁₈H₃₇, R⁴ is H, S is sulfur, R¹² is —C₁₆H₃₂CH₂CH₂—, p is 1, m is 8, T² is —COOH and D² is —NHC(O)O-L¹¹-O—, wherein L¹¹ is —CH₂CH₂—; and

wherein about 25% of the isocyanate groups of the polymeric isocyanate are reacted with acid groups.

The material of Example 6 (EX-6) is a fluorine-free compound that can represented by (SI-AA)₈-COOH/SA/PAPI/MEKO (0.6/0.6/3/1.8) and is derived from reaction, carried out in one or more steps, of component comprising:
(i) an acid functionalized isocyanate-reactive oligomer, having 8 repeating units;
(ii) a polymeric isocyanate, PAPI;
(iii) a monofunctional hydroxyl containing isocyanate-reactive compound, stearyl alcohol, SA; and
(iv) an isocyanate blocking agent, MEKO;

wherein the isocyanate-reactive oligomer has the formula (V), wherein Y¹ is H or an initiator residue, R¹ is C₁₈H₃₇—, R² is H, S is sulfur, R¹⁰ is —CH₂CH₂—, p is 1, m is 10, T¹ is —COOH and D is —N—; and

wherein about 25% of the isocyanate groups of the polymeric isocyanate are reacted with acid groups.

The material of Example 7 (EX-7) is a fluorine-free compound that can be represented by (SI-AA)₆-OH/BAc/PAPI/MEKO (0.6/0.6/3/1.8) and is derived from reaction carried out in one or more steps, of components comprising:
(i) a hydroxy functionalized isocyanate-reactive oligomer, having 6 repeating units;
(ii) a polymeric isocyanate, PAPI;
(iii) a monofunctional acid containing isocyanate-reactive compound, behenic acid, BAc; and
(iv) an isocyanate blocking agent, MEKO;

wherein the isocyanate-reactive oligomer has the formula (V), wherein Y is H or an initiator residue, R¹ is C₁₈H₃₇—, R² is H, S is sulfur, R¹⁰ is —CH₂CH₂—, p is 1, m is 10, T¹ is —OH— and D¹ is —NH—; and

wherein about 25% of the isocyanate groups of the polymeric isocyanate are reacted with acid groups.

Examples 3 to 7 were emulsified according to the procedure outlined above for Examples 1 and 2. The water-based dispersions of Examples 3 to 7 were applied to PES and PA fabrics according to the general procedure “Treatment Procedure via Padding Process, Water-based Dispersions” as given above. After the 10 or 20 washing cycles, the textile samples were dried in a Miele T-356 tumble drier, setting ‘Extra dry’. The treated samples were not ironed after tumble dry.

The treated fabrics were tested for their dynamic water repellent properties according to the “SprayRating (SR)” test outlined above. The samples were conditioned overnight at room temperature before testing. The results of spray rating were indicated as SR IN, SR 10L and SR 20L. The results are recorded in Table 2.

TABLE 2
Spray Ratings (1% SOF); Water-based Dispersions
(PES: 76.4 WPU; PA: 74.9% WPU)

| | |
|---|---|
| PES | PA |

| | SR | SR | SR | SR | SR | SR |
|---|---|---|---|---|---|---|
| Examples | IN | 10 L | 20 L | IN | 10 L | 20 L |
| EX-3 | 100 | 80 | 80 | 100 | 90 | 80 |
| EX-4 | 100 | 80 | 80 | 100 | 70 | 70 |
| EX-5 | 100 | 90 | 80 | 100 | 80 | 80 |
| EX-6 | 100 | 80 | 70 | 100 | 80 | 80 |
| EX-7 | 100 | 80 | 70 | 100 | 80 | 80 |

These results clearly demonstrate that fluorine-free treating compositions comprising the reaction product of a hydoxy or acid functionalized isocyanate-reactive oligomer with a polymeric isocyanate, a monofunctional isocyanate-reactive compound, and an isocyanate blocking agent provide treated textile substrates, not only with initial high water repellency, but surprisingly also good laundering resistance, after 10 or even after 20 laundering cycles, even without ironing.

### Comparative Example a (CE-A) and Comparative Example B (CE-B)

Comparative Example A (CE-A) and Comparative Example B (CE-B) were prepared according to the same procedure as for Example 3.

The material of Comparative Example A (CE-A) is a fluorine-free compound that can be represented by (SI-HOEA)₈-COOH/BAc/PAPI/MEKO (0.6/0.6/3/1.8).

The material of Comparative Example B (CE-B) is a fluorine free compound that can be represented by (SI-AA)₈-COOH/BAc/PAPI/MEKO (0.6/0.6/3/1.8)

The materials of Comparative Examples (CE-A) and (CE-B) are derived from reaction carried out in one or more steps, of components comprising:
(i) acid functionalized isocyanate-reactive oligomers, having 8 repeating units;
(ii) a polymeric isocyanate, PAPI;
(iii) a monofunctional acid-containing isocyanate-reactive compound, behenic acid, BAc; and
(iv) an isocyanate blocking agent, MEKO;

wherein the isocyanate-reactive oligomer of comparative Example (CE-A) has Formula (VI), wherein Y² is H or an initiator residue, R³ is C₁₈H₃₇—, R⁴ is H, S is sulfur, R¹² is —CH₂CH₂—, p is 1, m is 8, T² is —COOH and D² is —NHC(O)O-L¹¹-O— wherein L¹¹ is —CH₂CH₂—; and

wherein the isocyanate-reactive oligomer of comparative Example (CE-B) has the formula (V), wherein Y¹ is H or an initiator residue, R¹ is C₁₈H₃₇—, R² is H, S is sulfur, R¹⁰ is —CH₂CH₂—, p is 1, m is 10, T² is —OH and D¹ is —NH—; and

wherein about 40% of the isocyanate groups of the polymeric isocyanate are reacted with acid groups.

The reaction products were only partially soluble in the solvent used (ethylacetate) and therefore difficult to emulsify. The water repellency of these compounds was not determined.

### Example 8 (HOBA-SAc)₁₀—OH/BA/PAPI/MEKO (0.6/0.6/3/1.8)

In a three-necked flask of 500 ml fitted with a stirrer, heating mantle, an addition funnel and thermometer were placed 0.03 mol of (HOBA-SAc)₁₀—OH in EtOAc (as prepared above), 110 g EtOAc and 9.8 g (0.03 mol) Behenyl alcohol (BA). Using a Dean Stark trap, 20 g EtOAc were stripped off and the mixture was cooled to 50° C. under nitrogen atmosphere. Then 20.6 g (0.15 equiv) PAPI were added and the mixture was reacted at reflux (84C) for 24 hours. Then 7.8 g (0.09 mol) MEKO was added and the reaction was continued for 3 hours at 84° C. until no more isocyanate peak could be detected by IR. A clear, amber solution was obtained.

The material of Example 8 (EX-8) is a fluorine-free treating compound derived from reaction, carried out in one or more steps, of components comprising:
(i) a hydroxy functionalized isocyanate reactive oligomer, having 10 repeating units;
(ii) a polymeric isocyanate, PAPI;
(iii) a monofunctional acid containing isocyanate-reactive compound, behenic alcohol, BA; and
(iv) an isocyanate blocking agent, MEKO;

wherein the isocyanate reactive oligomer has Formula (V), wherein Y¹ is H or an initiator residue, R¹ is C₁₇H₃₅, R² is H, S is sulfur, R¹⁰ is —CH₂CH₂—, p is 1, m is 10, T¹ is —OH and D¹ is —C(O)O-L¹-O, wherein L^{J} is —(CH₂)₄—; and

wherein none of the isocyanate groups of the polymeric isocyanate are reacted with acid and/or amide groups.

### Examples 9 to 12

Examples 9 to 12 were all made according to the same procedure as given for Example 8.

The material of Example 9 (EX-9) is a fluorine-free treating compound that can be represented by (HOBA-BAc)₁₀—OH/BA/PAPI/MEKO (0.6/0.6/3/1.8) and is derived from reaction, carried out in one or more steps, of components comprising:
(i) a hydroxy functionalized isocyanate-reactive oligomer, having 10 repeating units;
(ii) a polymeric isocyanate, PAPI;
(iii) a monomeric hydroxyl containing isocyanate-reactive compound, behenyl alcohol, BA; and
(iv) an isocyanate blocking agent, MEKO;

wherein the isocyanate reactive oligomer is according to Formula (V), wherein Y¹ is H or an initiator residue, R¹ is C₂₁H₄₃, R² is H, S is sulfur, R¹⁰ is —CH₂CH₂—, p is 1, m is 10, T¹ is —OH, and D¹ is —C(O)O-L¹-O, wherein L¹ is —(CH₂)₄—; and

wherein none of the isocyanate groups of the polymeric isocyanate are reacted with acid and/or amide groups.

The material of Example 10 (EX-10) is a fluorine-free compound that can be represented by (HOBA-UNICID 350)₄-OH/BA/PAPI/MEKO (0.6/0.6/3/1.8) and is derived from reaction carried out in one or more steps, of components comprising:
(i) a hydroxy functionalized isocyanate-reactive oligomer, having 4 repeating units;
(ii) a polymeric isocyanate, PAPI;
(iii) a monofunctional alcohol containing isocyanate-reactive compound, behenyl alcohol, BA; and
(iv) an isocyanate blocking agent, MEKO;

wherein the isocyanate reactive oligomer is according to Formula (V), wherein Y¹ is H or an initiator residue, R¹ is CₙH₂ₙ₊₁; n=about 30, R² is H, S is sulfur, R¹⁰ is —CH₂CH₂—, p is 1, in is 4, T¹ is —OH— and D¹ is —C(O)O-L¹-O, wherein U is —(CH₂)₄—; and

wherein none of the isocyanate groups of the polymeric isocyanate are reacted with acid and/or amide groups.

The material of Example 11 (EX-11) is a fluorine-free compound that can be represented by (UNILIN 350/SAnh/HOBA)₄-OH/BA/PAPI/MEKO (0.6/0.6/3/1.8) and is derived from reaction carried out in one or more steps, of components comprising:
(i) a hydroxy functionalized isocyanate-reactive oligomer, having 4 repeating units;
(ii) a polymeric isocyanate, PAPI;
(iii) a monofunctional alcohol containing isocyanate-reactive compound, behenyl alcohol, BA; and
(iv) an isocyanate blocking agent, MEKO;

wherein the isocyanate reactive oligomer is according to Formula (V), wherein Y¹ is H or an initiator residue, R¹ is CₙH₂ₙ₊₁, wherein n=about 30, R² is H, S is sulfur, R¹⁰ is —CH₂CH₂—, p is 1, is 4, T¹ is —OH and D¹ is —OC(O)-L³-C(O)O-L⁴-O, wherein L is —(CH₂)₂— and L⁴ is —(CH₂)₄—; and

wherein none of the isocyanate groups of the polymeric isocyanate are reacted with acid and/or amide groups.

The material of Example 12 (EX-12) is a fluorine-free treating composition that can be represented by (HOEA/UNICID 350)₄-OH/BA/PAPI/MEKO (0.6/0.6/3/1.8) and wherein the compound is derived from a reaction carried out in one or more steps, of components comprising:
(i) a hydroxy functionalized isocyanate-reactive oligomer, having 4 repeating units;
(ii) a polymeric isocyanate, PAPI;
(iii) a monofunctional alcohol containing isocyanate-reactive compound, behenyl alcohol, BA; and
(iv) an isocyanate blocking agent, MEKO;

wherein the isocyanate reactive oligomer is according to Formula (V), wherein Y¹ is H or an initiator residue, R¹ is CₙH₂ₙ₊₁; n=about 30, R² is H, S is sulfur, R¹⁰ is —CH₂CH₂—, p is 1, m is 4, T¹ is —OH and D¹ is —C(O)O-L¹- wherein is —(CH₂)₄—; and

wherein none of the isocyanate groups of the polymeric isocyanate are reacted with acid and/or amide groups.

Examples 8 to 12 were emulsified according to the procedure outlined above for Examples 1 and 2. The water-based dispersions of Examples 8 to 11 were applied to PES and PA fabrics according to the general procedure “Treatment Procedure via Padding Process. Water-based Dispersions” as given above. After the 10 or 20 washing cycles, the textile samples were dried in a Miele T-356 tumble drier, setting ‘Extra dry’. The treated samples were ironed after tumble dry.

The treated fabrics were tested for their dynamic water repellent properties according to the “Spray Rating (SR)” test outlined above. The samples were conditioned overnight at room temperature before testing. The results of spray rating were indicated as SR IN, SR 10L and SR 20L. The results are recorded in Table 3.

TABLE 3
Spray Ratings (1% SOF); Water-based Dispersions
(PES: 76.4 WPU; PA: 74.9% WPU)

| | PES | | | PA | | |
|---|---|---|---|---|---|---|
| | SR | SR | SR | SR | SR | SR |
| Examples | IN | 10 L IR | 20 L IR | IN | 10 L IR | 20 L IR |
| EX-8 | 90 | 80 | 70 | 90 | 80 | 70 |
| EX-9 | 100 | 70 | 70 | 100 | 70 | 50 |
| EX-10 | 100 | 80 | 80 | 100 | 80 | 70 |
| EX-11 | 100 | 90 | 70 | 100 | 80 | 70 |
| EX-I2 | 100 | 80 | 80 | 100 | 80 | 70 |

These results clearly demonstrate that fluorine-free treating compositions comprising the reaction product of a hydroxy functionalized isocyanate-reactive oligomer with a polymeric isocyanate, a monofunctional isocyanate-reactive compound, and an isocyanate blocking agent provide treated textile substrates, not only with initial high water repellency, but surprisingly also good laundering resistance, after 10 or even after 20 laundering cycles.

### Example 13 (ST-AM)₆-OH/SA/PAPI/MEKO (0.6/0.6/3/1.8)

In a three-necked flask of 500 ml fitted with a stirrer, heating mantle, an addition funnel and thermometer were placed 0.033 mol of (SI-AM)₆-OH in EtOAc (as prepared above) and 70 g EtOAc. The mixture was heated to 70° C. under nitrogen atmosphere. Then 8.9 g (0.033 mol) SA and 22.5 g (0.165 equiv) PAPI were added as well as 1 drop of DBTDL. The reaction was continued for about 16 hours at 85° C. Then 8.6 g (0.099 mol) MEKO was added dropwise and the reaction was continued for 2 hours until no more isocyanate peak could be detected by FTIR. A clear, amber solution was obtained

The material of Example 13 (EX-13) is a fluorine-free compound derived from reaction carried out in one or more steps, of components comprising:
(i) a hydroxy functionalized isocyanate-reactive oligomer, having 6 repeating units;
(ii) a polymeric isocyanate, PAPI;
(iii) a monofunctional hydroxyl containing isocyanate-reactive compound, stearyl alcohol, SA; and
(iv) an isocyanate blocking agent, MEKO;

wherein the isocyanate reactive oligomer is according to Formula (V), wherein Y¹ is H or an initiator residue, R¹ is C₁₈C₃₇, R² is H, S is sulfur, R¹⁰ is —CH₂CH₂—, p is 1, m is 6, T¹ is —OH and D¹ is —NH—C(O)—NH—; and

wherein none of the isocyanate groups of the polymeric isocyanate are reacted with acid and/or amide groups.

### Examples 14 to 18

Examples 14 to 18 were all made according to the same procedure as given for Example 13.

The material of Example 14 (EX-14) is a fluorine-free compound that can be represented by (SI-MAM)₆-OH/SA/PAPI/MEKO (0.6/0.6/3/1.8) and is derived from reaction, carried out in one or more steps, of components comprising:
(i) a hydroxy functionalized isocyanate-reactive oligomer, having 6 repeating units;
(ii) a polymeric isocyanate, PAPI;
(iii) a monomeric hydroxyl containing isocyanate-reactive compound, stearyl alcohol, SA; and
(iv) an isocyanate blocking agent, MEKO;

wherein the isocyanate reactive oligomer is according to Formula (V), wherein Y¹ is H or an initiator residue, R¹ is C₁₈H₃₇, R² is CH₃, S is sulfur, R¹⁰ is —CH₂CH₂—, p is 1, m is 6, T¹ is —OH and D¹ is NH—C(O)—NH—; and

wherein none of the isocyanate groups of the polymeric isocyanate are reacted with acid and/or amide groups.

The material of Example 15 (EX-15) is a fluorine-free treating composition that can be represented by (SI-HOEA)₆-OH/SAmd/PAPI/MEKO (0.6/0.6/3/1.8) and wherein the compound is derived from a reaction carried out in one or more steps, of components comprising mixture of:
(i) a hydroxy functionalized isocyanate-reactive oligomer, having 6 repeating units, made by the radical initiated reaction of 6 moles of an acrylate comprising a urethane group (and comprising a hydrocarbon group having 18 carbon atoms), in the presence of 1 mole of 2-mercaptoethanol;
(ii) a polymeric isocyanate, PAPI;
(iii) a monofunctional amide containing isocyanate-reactive compound, stearyl amide, SAmd; and
(iv) an isocyanate blocking agent, MEKO;

wherein the isocyanate reactive oligomer is Formula (VI) wherein Y² is H or an initiator residue, R³ is C₁₈H₃₇, R⁴ is H, S is sulfur, R¹² is —CH₂CH₂—, p is 1, m is 6, T² is —OH and D² is —NHC(O)O-L¹¹-O—, wherein L¹¹ is —CH₂CH₂; and

wherein about 25% of the isocyanate groups of the polymeric isocyanate are reacted with amide groups.

The material of Example 16 (EX-16) is a fluorine-free treating composition that can be represented by (SI-AM)₆-COOH/SA/PAPI/MEKO (0.6/0.6/3/1.8) and wherein the compound is derived from a reaction carried out in one or more steps, of components comprising:
(i) an acid functionalized isocyanate-reactive oligomer having 6 repeating units;
(ii) a polymeric isocyanate, PAPI;
(iii) a monomeric hydroxyl containing isocyanate-reactive compound, stearyl alcohol, SA; and
(iv) an isocyanate blocking agent, MEKO;

wherein the isocyanate reactive oligomer is Formula (V), wherein Y¹ is H or an initiator residue, R¹ is C₁₈H₃₇, R² is H, S is sulfur, R⁵ is —CH₂CH₂—, p is 1 is 6 T¹ is-COOH and D¹ is —NH—C(O)—NH—; and

wherein about 25% of the isocyanate groups of the polymeric isocyanate are reacted with acid groups.

The material of Example 17 (EX-17) is a fluorine-free treating composition that can be represented by (SCl-AM)₆-OH/SA/PAPI/MEKO (0.6/0.6/3/1.8) and wherein the compound is derived from a reaction carried out in one or more steps, of components comprising mixture of:
(i) a hydroxy functionalized isocyanate-reactive oligomer, having 6 repeating units;
(ii) a polymeric isocyanate, PAPI;
(iii) a monomeric hydroxyl containing isocyanate-reactive compound, stearyl alcohol, SA; and
(iv) an isocyanate blocking agent, MEKO;

wherein the isocyanate reactive oligomer is Formula (V), wherein Y¹ is H or an initiator residue, R¹ is C₁₇H₃₅, R² is H, S is sulfur, R⁵ is —CH₂CH₂—, p is 1, m is 6, T¹ is —OH and D¹ is —C(O)—NH—; and

wherein none of the isocyanate groups of the polymeric isocyanate are reacted with acid and/or amide groups.

The material of Example 18 (EX-18) is a fluorine-free treating compound that can be represented by (SCI-MAM)₆-OH/SA/PAPI/MEKO (0.6/0.6/3/1.8) and is derived from reaction carried out in one or more steps, of components comprising:
(i) a hydroxy functionalized isocyanate-reactive oligomer, having 6 repeating units;
(ii) a polymeric isocyanate, PAPI;
(iii) a monomeric hydroxyl containing isocyanate-reactive compound, stearyl alcohol, SA; and
(iv) an isocyanate blocking agent, MEKO;

wherein the isocyanate reactive oligomer is Formula (V), wherein Y¹ is H or an initiator residue, R¹ is C₁₇H₃₅, R² is CH₃, S is sulfur, R⁵ is —CH₂CH₂—, p is 1, m is 6, T¹ is —OH and D¹ is —C(O)—NH—; and

wherein none of the isocyanate groups of the polymeric isocyanate are reacted with acid and/or amide groups.

Examples 13 to 18 were emulsified according to the procedure outlined above for Examples 1 and 2. The water-based dispersions of Examples 13 to 18 were applied to PES and PA fabrics according to the general procedure “Treatment Procedure via Padding Process, Water-based Dispersions” as given above. After the 10 or 20 washing cycles, the textile samples were dried in a Miele T-356 tumble drier, setting ‘Extra dry’. The treated samples were not ironed after tumble dry.

The treated fabrics were tested for their dynamic water repellent properties according to the “Spray Rating (SR)” test outlined above. The samples were conditioned overnight at room temperature before testing. The results of spray rating were indicated as SR IN, SR 10L and SR 20L. The results are recorded in Table 4.

TABLE 4
Spray Ratings (1% SOF); Water-based Dispersions
(PES: 76.4 WPU; PA: 74.9% WPU)

| | PES | | | PA | | |
|---|---|---|---|---|---|---|
| Examples | SR IN | SR 10 L | SR 20 L | SR IN | SR 10 L | SR 20 L |
| EX-13 | 100 | 80 | 80 | 100 | 80 | 80 |
| EX-14 | 100 | 70 | 70 | 100 | 70 | 70 |
| EX-15 | 100 | 90 | 90 | 100 | 80 | 80 |
| EX-16 | 100 | 80 | 70 | 100 | 70 | 70 |
| EX-17 | 100 | 70 | 70 | 100 | 70 | 70 |
| EX-18 | 100 | 70 | 70 | 100 | 80 | 80 |

These results clearly demonstrate that fluorine-free treating compositions comprising the reaction product of a hydroxy or acid-functionalized isocyanate-reactive oligomer with a polymeric isocyanate, a mono functional isocyanate-reactive compound, and an isocyanate blocking agent provides treated textile substrates, not only with initial high water repellency, but surprisingly also good laundering resistance, after 10 or even after 20 laundering cycles. The results further indicate that it is not necessary to iron the treated samples after extended laundering cycles.

### Example 19 (SI-AA)₈-OH/MDI 1/4 (Polycarbodiimide ‘PCD’)

A 500 ml reaction flask was loaded with 0.025 mol (SI-AA)₈-OH oligomer in EtOAc as prepared above. 0.1 mol (25 g) MDI and 15 g EtOAc were added, together with 0.1 g MPPO catalyst. The reaction mixture was heated to 85-88° C. for 24 hours under liberation of CO₂. FTIR analysis indicated that all isocyanate groups were converted into carbodiimide groups. The reaction mixture was then diluted to 50% solids in EtOAc.

The material of Example 19 (EX-19) comprises at least one polycarbodiimide compound derived from a carbodiimidization reaction, carried out in one or more steps, of components comprising:
(i) a hydroxy functionalized isocyanate-reactive oligomer comprising 8 repeating units; and
(ii) a polyisocyanate, MDI;

wherein the isocyanate-reactive oligomer is Formula (V), wherein Y¹ is H or an initiator residue, R¹ is C₁₈H₃₇, R² is H, S is sulfur, R¹⁰ is —CH₂CH₂—, p is 1, in is 6, 8 or 10, T¹ is —OH and D¹ is —NH—.

### Example 20 (SI-MA)₈-OH/MDI 1/4 (PCD)

Example 20 (EX-20) was made according to the same procedure as given for Example 19 but starting from (SI-MA)₈-OH.

The material of Example 20 comprises at least one polycarbodiimide compound derived from a carbodiimidization reaction, carried out in one or more steps, of components comprising:
(i) a hydroxy functionalized isocyanate-reactive oligomer comprising 8 repeating units; and
(ii) a polyisocyanate, MDI;

wherein the isocyanate-reactive oligomer is Formula (V), wherein Y¹ is H or an initiator residue, R¹ is C₁₈H₃₇, R² is CH₃, S is sulfur, R¹⁰ is —CH₂CH₂—, p is 1, m is 6, 8 or 10, T¹ is —OH— and D¹ is —NH—.

The materials of Examples 19 and 20 were emulsified according to the procedure outlined above for Examples 1 and 2. The water-based dispersions of Examples 19 and 20 were applied to PES and PA fabrics according to the general procedure “Treatment Procedure via Padding Process, Water-based Dispersions” as given above. After the 10 or 20 washing cycles the textile samples were dried in a Miele T-356 tumble drier, setting ‘Extra dry’. After 20 washing cycles, the treated samples were ironed after tumble dry.

The treated fabrics were tested for their dynamic water repellent properties according to the “Spray Rating (SR)” test outlined above. The samples were conditioned overnight at room temperature before testing. The results of spray rating were indicated as SR IN (initial) and SR 10L, SR 20L and SR 20L IR. The results are recorded in Table 5.

TABLE 5
Spray Ratings (0.6% SOF); Water-based Dispersions (PES: 100% WPU; PA: 93% WPU)

| | PES | | | | PA | | | |
|---|---|---|---|---|---|---|---|---|
| Example | SR IN | SR 10 L | SR 20 L | SR 20 L IR | SR IN | SR 10 L | SR 20 L | SR 20 L IR |
| EX-19 | 100 | 80 | 80 | 80 | 100 | 100 | 90 | 90 |
| EX-20 | 100 | 90 | 90 | 90 | 100 | 90 | 90 | 90 |

These results clearly demonstrate that fluorine-free treating compositions comprising the polycarbodiimide reaction product of a hydroxy functionalized isocyanate-reactive oligomer (of a (meth)acrylamide monomer comprising a hydrocarbon group having IS carbon atoms) with a diisocyanate, provide treated textile substrates, not only with initial high water repellency, but surprisingly also good laundering resistance, after 10 or even after 20 laundering cycles. The results further indicate that it is not necessary to iron the treated samples after extended laundering cycles.

### Example 21 (HOBA-BAc)₁₀—OH/MDI 1/4 (PCD)

In a three-necked flask of 500 ml, fitted with a stirrer, condenser and heating mantle, were placed 0.03 mol of the hydroxy functionalized (HOBA-BAc)₁—OH, prepared as described above, together with 110 g EtOAc. Using a Dean-Stark trap, 20 g EtOAc was stripped off and the mixture was cooled to about 50° C. under nitrogen atmosphere. Then 30.0 g (0.12 equivalents) of MDT were added and the mixture was reacted at reflux (84° C.) for 4 hours. Then 0.15 g MPPO catalyst was added and the reaction continued for 16 hours. A slightly hazy, yellow solution was obtained. IR indicated that all NCO had reacted and that carbodiimide groups were formed

The material of Example 21 comprises at least one polycarbodiimide compound derived from a carbodiimidization reaction, carried out in one or more steps, of components comprising:
(i) a hydroxy functionalized isocyanate-reactive oligomer comprising 10 repeating units; and
(ii) a polyisocyanate, MDI;

wherein the isocyanate-reactive oligomer is Formula (V), wherein Y¹ is H or an initiator residue, R¹ is C₂₁H₄₃, R² is H, S is sulfur, R¹⁰ is —CH₂CH₂—, p is 1, in is 10, T¹ is —OH, and D¹ is —C(O)O-L¹-O, wherein L¹ is —(CH₂)₄—.

### Example 22 (HOBA-UNICID 350)₄-OH/MDI 1/4 (PCD)

Example 22 (HOBA-UNICID 350)₄-OH/MDI 1/4 was made according to the same procedure as Example 21, but starting from the hydroxy functionalized oligomer (HOBA-UNICID 350)₄—OH.

The material of Example 22 comprises at least one polycarbodiimide compound derived from a carbodiimidization reaction, carried out in one or more steps, of components comprising:
(i) a hydroxy functionalized isocyanate-reactive oligomer comprising 4 repeating units; and
(ii) a polyisocyanate, MDI;

wherein the isocyanate-reactive oligomer is Formula (V), wherein Y¹ is H or an initiator residue, R¹ is CₙH₂ₙ₊₁; n=about 30, R² is H, S is sulfur, R¹⁰ is —CH₂—CH₂—, P is 1, m is 4, T¹ is —OH and D¹ is —C(O)O-L¹-O, wherein L¹ is —(CH₂)₄—.

Examples 21 and 22 were emulsified according to the procedure outlined above for Examples 1 and 2. The water-based dispersions of Examples 21 and 22 were applied to PES and PA fabrics according to the general procedure “Treatment Procedure via Padding Process, Water-based Dispersions” as given above. After the 10 or 20 washing cycles, the textile samples were dried in a Miele T-356 tumble drier, setting ‘Extra dry’. The treated samples were ironed after tumble dry.

The treated fabrics were tested for their dynamic water repellent properties according to the “Spray Rating (SR)” test outlined above. The samples were conditioned overnight at room temperature before testing. The results of spray rating were indicated as SR IN (initial) and SR 10L IR and SR 20L IR. The results are recorded in Table 6.

TABLE 6
Spray Ratings (1% SOF); Water-based Dispersions
(PES: 76.4 WPU; PA: 74.9% WPU)

| | PES | | | PA | | |
|---|---|---|---|---|---|---|
| | SR | SR | SR | SR | SR | SR |
| Examples | IN | 10 L IR | 20 L IR | IN | 10 L IR | 20 L IR |
| EX-21 | 100 | 80 | 70 | 90 | 80 | 70 |
| EX-22 | 100 | 80 | 80 | 90 | 80 | 80 |

These results clearly demonstrate that fluorine-free treating compositions comprising the polycarbodiimide reaction product of a hydroxy functionalized isocyanate-reactive oligomer with a diisocyanate provide treated textile substrates, not only with initial high water repellency, but surprisingly also good laundering resistance, after 10 or even after 20 laundering cycles.

### Example 23 (SI-HOEA)₈-COOH/MDI 1/4 (PCD)

In a three-necked flask of 500 ml, fitted with a stirrer, condenser and heating mantle, were placed 0.03 mol of (S-HOEA)₈-COOH, prepared as described above, 30 g (0.12 mol) MDI, 0.07 g MgCl2 and 65 g under a nitrogen atmosphere. The reaction mixture was heated up to 80° C. under liberation of CO₂ due to the amide formation. The reaction was continued until no more CO₂ was released (after about 3 hours). Then 0.1 g MPPO catalyst was added and the reaction was continued for 24 hours at 85° C. More CO₂ evolution was observed due to the formation of the polycarbodiimide. A hazy solution was obtained. IR analysis indicated that all isocyanate groups were reacted and carbodiimides were formed.

The material of Example 23 comprises at least one polycarbodiimide compound derived from a carbodiimidization reaction, carried out in one or more steps, of components comprising:
(i) an acid functionalized isocyanate-reactive oligomer comprising 8 repeating units; and
(ii) a polyisocyanate, MDT;

wherein the isocyanate-reactive oligomer is VI, wherein Y² is H or an initiator residue, R³ is C₁₈H₃₇—, R⁴ is H, S is sulfur, R¹² is —CH₂CH₂—, p is 1, in is 8, T² is —COOH and D² is —NHC(O)O-L¹¹-O—, wherein L¹¹ is —CH₂CH₂—.

### Example 24 (SI-AA)₈-COOH/MDI 1/4 (PCD)

Using the same procedure as for Example 24, (SI-AA)₈-COOH/MDI 1/4 was prepared, but using (ST-AA)₈-COOH instead of (SI-HOEA)₈-COOH.

The material of Example 24 comprises at least one polycarbodiimide compound derived from a carbodiimidization reaction, carried out in one or more steps, of components comprising:
(i) an acid functionalized isocyanate-reactive oligomer comprising 8 repeating units; and
(ii) a polyisocyanate, MDI;

wherein the isocyanate-reactive oligomer Formula (V) wherein Y¹ is H or an initiator residue, R¹ is C₁₈H₃₇, R² is H, S is sulfur, R¹⁰ is —CH₂CH₂— p is 1, m is 8, T¹ is —COOH and D¹ is —NH—.

### Example 25 (ODA)₈-COOH/isoSA/MDI 1/1/4

In a three-necked flask of 500 ml, fitted with a stirrer, condenser and heating mantle, were placed 0.03 mol (ODA)₈-COOH, prepared as described above, 30 g (0.12 mol) MDI 0.07 g MgCl₂ and 65 g under a nitrogen atmosphere. The reaction mixture was heated up to 80° C. under liberation of CO₂ due to the amide formation. The reaction was continued until no more CO₂ was released (after about 3 hours). Then, 8.1 g (0.03 mol) of isoSA were added and reacted for 4 hours at 85° C. Then 0.1 g MPPO catalyst was added and the reaction was continued for 24 hours at 85° C. More CO₂ evolution was observed due to the formation of the polycarbodiimide. A hazy solution was obtained. IR analysis indicated that all isocyanate groups were reacted and carbodiimides were formed.

The material of Example 25 comprises at least one polycarbodiimide compound derived from a carbodiimidization reaction, carried out in one or more steps, of components comprising:
(i) an acid functionalized isocyanate-reactive oligomer comprising 8 repeating units;
(ii) a polyisocyanate, MDI; and
(iii) an additional monofunctional isocyanate-reactive compound, isostearyl alcohol, isoSA;
   wherein the isocyanate-reactive oligomer is Formula (VI), wherein Y² is H or an initiator residue, R³ is C₁₈H₃₇—, R⁴ is H, S is sulfur, R¹² is —CH₂CH₂—, p is 1, m is 8, T² is —COOH and D² is —O—.

Examples 23 to 25 were emulsified according to the procedure outlined above for Examples 1 and 2. The water-based dispersions of Examples 23 to 25 were applied to PES and PA fabrics according to the general procedure “Treatment Procedure via Padding Process, Water-based Dispersions” as given above. After the 10 or 20 washing cycles, the textile samples were dried in a Miele T-356 tumble drier, setting ‘Extra dry’. The treated samples were not ironed after tumble dry.

The treated fabrics were tested for their dynamic water repellent properties according to the “Spray Rating (SR)” test outlined above. The samples were conditioned overnight at room temperature before testing. The results of spray rating were indicated as SR IN, SR 10L and SR 20L. The results are recorded in Table 7

TABLE 7
Spray Ratings (1% SOF); Water-based Dispersions
(PES: 76.4 WPU; PA: 74.9% WPU)

| | |
|---|---|
| PES | PA |

| | SR | SR | SR | SR | SR | SR |
|---|---|---|---|---|---|---|
| Examples | IN | 10 L | 20 L | IN | 10 L | 20 L |
| EX-23 | 100 | 90 | 80 | 100 | 80 | 80 |
| EX-24 | 100 | 80 | 80 | 100 | 80 | 80 |
| EX-25 | 90 | 80 | 80 | 90 | 80 | 80 |

These results clearly demonstrate that fluorine-free treating compositions comprising the polycarbodimide reaction product of an acid functionalized isocyanate-reactive oligomer (of a monomer comprising a hydrocarbon group having 18 carbon atoms) with a diisocyanate, and optional a monofunctional isocyanate-reactive compound (EX-25) provide treated textile substrates, not only with initial high water repellency, but surprisingly also good laundering resistance, after 10 or even after 20 laundering cycles.

### Example 26 and 27 and Comparative Examples CE-C to CE-F

### Example 26 Poly (SI-AA) (or p(SI-AA))

In a 1 liter round-bottomed 3 necked reaction flask were placed 72 g acrylic acid (1 mol), 108 g ethylacetate, 0.1 g MEHQ, 0.1 g PTZ and 0.1 g MgCl₂. This mixture was heated to 70° C. and, over a 1 hour period, 300 g stearylisocyanate was added (appox. 1 mol). As soon as the stearyisocyanate was added the formation of CO was observed. After all stearylisocyanate was added, the reaction was continued for 2 hours at reflux temperature. At the end of this reaction period, no more CO₂ was formed.

FTIR showed that all NCO was reacted, and NMR showed that more than 90% N-stearyl-acrylamide (i.e. C₁₈H₃₇NH—C(O)CH═CH₂) was formed which is referred to as SI-AA. The ethylacetate was then removed by vacuum distillation on a rotavapor. The solvent free monomer SI-AA was a white hard waxy material at room temperature.

In a second step, 80 g SI-AA, 2.1 g Ethoquad C12, 4.8 g Tergitol TMN-624 g Tergitol 15-S-30, 28 g Propyleneglycol and 135 g D.I. Water was mixed and heated to 85° C. in a glass beaker. This mixture was dispersed by ultrasonication for 5 minutes at maximum setting with a Branson sonifier. The monomer dispersion was then transferred into a 3-necked round bottomed flask, and purged with nitrogen for 2 minutes at 85° C. After the oxygen was removed from the flask, 0.2 g Wako V-50 initiator was added and a moderate exotherm to 95° C. was observed. After 30 minutes reaction 0.2 g V-50 was added, and the reaction continued for 2 hrs at 85° C., and then the dispersion was cooled to room temperature and filtered. The dispersion was then diluted with DI-water to 25% solids. The material obtained is referred to as “p(SI-AA)”.

Using the same procedure, p(SI-MA) (EX-27) was made, but starting from MA instead of AA.

p(ST-AA) and p(ST-MA) are polymeric compounds derived from the polymerization of at least one acrylamide monomer according to Formula (I) wherein R is C₁₈H₃₇—, D is —NH— and R² is —H or —CH₃ respectively.

The materials of Comparative Examples CE-C to CE-F were prepared according to the same procedure, but starting from octadecyl acrylate (CE-C; polyoctadecyl acrylate or p(ODA)), octadecyl methacrylate (CE-D; polyoctadecyl methacrylate or p(ODMA)), behenyl acrylate (CEE; polybehenyl acrylate or p(BEA)) and behenyl methacrylate (CE-F; polybehenyl methacrylate or p(BEMA).

p(ODA), p(ODMA), p(BEA) and p(BEMA) are polymeric compounds derived from the polymerization of at least one acrylate monomer according to the Formula (II), wherein: R³ represents a branched or straight chain hydrocarbon group having from 18 or 21 carbon atoms; R⁴ represents H or CH₃; and D² represents —O—.

Before application of the dispersion to textile fabric by pad application, the polymer dispersions of Examples EX-26 and EX-27 and the comparative Examples CE-C to CE-F were further diluted with DI Water to a concentration of 20 g/liter and were then applied to dark grey polyester (PES) and grey polyamide (PA) microfiber fabrics according to the general procedure “Treatment Procedure via Padding Process, Water-based Dispersions” as given above. After application of the treatment solutions, the fabrics were dried and cured for 2 minutes at 150° C., and conditioned overnight at room temperature before testing.

The treated fabrics were tested for their initial dynamic water repellent properties according to the “Spray Rating (SR)” test outlined above. The samples were conditioned overnight at room temperature before testing. The results of spray rating were indicated as SR IN. The results are recorded in Table 8.

TABLE 8
Spray Ratings (1% SOF); Water-based Dispersions
(PES: 100 WPU; PA: 93% WPU)

| | PES (100% WPU) | PA (93% WPU) |
|---|---|---|
| Example | SR IN | SR IN |
| EX-26 | 100 | 100 |
| EX-27 | 90 | 100 |
| CE-C | 0 | 0 |
| CE-D | 0 | 0 |
| CE-E | 0 | 0 |
| CE-F | 0 | 0 |

These results clearly demonstrate that the polymeric materials, made from the polymerization of alkyl (meth)acrylamides having a hydrocarbon group comprising 18 carbon atoms, outperform the materials made with alkyl (meth)acrylates having a hydrocarbon group comprising 18 or 21 carbon atoms (CE-C, CE-D, CE-E, and CE-F).

### Examples 28 to 31

Examples 28 to 31 were made with blends of polycarbodiimides and polyacrylate as indicated in table 9. The water based blends were applied to PES and PA fabrics according to the general procedure “Treatment Procedure via Padding Process, Water-based Dispersions” as given above. The treated samples were not ironed after tumble dry.

The treated fabrics were tested for their dynamic water repellent properties according to the “Spray Rating (SR)” test outlined above. The properties were tested initially and after 10 or 20 launderings. After the 10 or 20 washing cycles the textile samples were dried in a Miele T-356 tumble drier, setting ‘Extra dry’. The samples were not ironed. The samples were conditioned overnight at room temperature before testing. The results of spray rating are recorded SR IN, SR 10L and SR 20L. The results are recorded in Table 10.

TABLE 9
Fluorine free treatment compositions comprising
blends of polycarbodiimide and polyacrylate

| | Polycarbodiimide | | Polyacrylate | |
|---|---|---|---|---|
| Example | Composition | Wt % | Composition | Wt % |
| EX-28 | (SI-HOEA)₈- | 35 | p (SI-HOEA) | 65 |
| | COOH/MDI 1/4 | | | |
| EX-29 | (SI-AA)8COOH/ | 35 | p (SI-HOEA) | 65 |
| | MDI 1/4 | | | |
| EX-30 | (SI-AM)6-OH/ | 35 | p (SI-HOEA) | 65 |
| | MDI 1/4 | | | |
| EX-31 | (SI-MAM)6-OH/MDI 1/4 | 35 | p (SI-HOEA) | 65 |

TABLE 10
Spray Ratings (1% SOF); Water-based Dispersions
(PES: 76.4 WPU; PA: 74.9% WPU)

| | PES | | | PA | | |
|---|---|---|---|---|---|---|
| Examples | SR IN | SR 10 L | SR 20 L | SR IN | SR 10 L | SR 20 L |
| EX-28 | 100 | 90 | 90 | 100 | 90 | 90 |
| EX-29 | 100 | 90 | 90 | 100 | 80 | 80 |
| EX-30 | 100 | 90 | 90 | 100 | 90 | 90 |
| EX-31 | 100 | 90 | 90 | 100 | 80 | 80 |

These results clearly demonstrate that fluorine-free treating compositions comprising blends of polycarbodiimides and polyacrylates provide treated textile substrates, not only with initial high water repellency, but surprisingly also good laundering resistance after 10 or even after 20 laundering cycles.

### Examples 32 to 34

Examples 32 to 34 were made with blends of polyacrylates and blocked polyisocyanates as indicated in Table 11.

The water based blends were applied to PES and PA fabrics at 1% SOF, according to the general procedure “Treatment Procedure via Padding Process, Water-based Dispersions” as given above. The treated fabrics were tested for their dynamic water repellent properties according to the “Spray Rating (SR)” test outlined above. The properties were tested initially and after 10 or 20 launderings. After the 10 or 20 washing cycles, the textile samples were dried in a Miele T-356 tumble drier, setting ‘Extra dry’. The samples were not ironed. The samples were conditioned overnight at room temperature before testing. The results of spray rating were indicated as SR IN SR 10L and SR 20L. The results are recorded in Table 12.

TABLE 11
Fluorine free treatment compositions comprising blends
of blocked polyisocyanates and polyacrylate

| | Blocked polyisocyanates | | Polyacrylate | |
|---|---|---|---|---|
| Example | Composition | Wt % | Composition | Wt % |
| EX-32 | (SI-AA)₈- | 35 | p (SI-HOEA) | 65 |
| | OH/SA/PAPI/MEKO | | | |
| | 0.6/0.6/3/1.8 | | | |
| EX-33 | (SI-AM)₈- | 35 | p (SI-HOEA) | 65 |
| | OH/SA/PAPI/MEKO | | | |
| | 0.6/0.6/3/1.8 | | | |
| EX-34 | (SCI-AM)₈- | 35 | p (SI-HOEA) | 65 |
| | OH/SA/PAPI/MEKO | | | |
| | 0.6/0.6/3/1.8 | | | |

TABLE 12
Spray Ratings (1% SOF); Water-based Dispersions
(PES: 76.4 WPU; PA: 74.9% WPU)

| | PES | | | PA | | |
|---|---|---|---|---|---|---|
| Examples | SR IN | SR 10 L | SR 20 L | SR IN | SR 10 L | SR 20 L |
| EX-32 | 100 | 80 | 80 | 100 | 90 | 80 |
| EX-33 | 100 | 90 | 80 | 100 | 80 | 80 |
| EX-34 | 100 | 80 | 70 | 100 | 80 | 80 |

These results clearly demonstrate that fluorine-free treating compositions comprising blends of polyacrylates and blocked polyisocyanates provide treated textile substrates, not only with initial high water repellency, but surprisingly also good laundering resistance, after 10 or even after 20 laundering cycles.

### Example 35

Examples 35 was made with a fluorine free treatment composition comprising a blend of polyacrylates polycarbodiimide and blocked polyisocyanates. The fluorine-free treatment composition comprised 35 Wt % p(SI-HOEA), 35 Wt % (SI-AA)₈OH/SA/PAPI/MEKO (06/0.6/3/1.8) and 30 Wt % (SI-AM)₆-OH/MDI (1/4).

The water based blends were applied to PES and PA fabrics at 1% SOF, according to the general procedure “Treatment Procedure via Padding Process, Water-based Dispersions” as given above. The treated fabrics were tested for their dynamic water repellent properties according to the “Spray Rating (SR)” test outlined above. The properties were tested initially and after 10 or 20 launderings. After the 10 or 20 washing cycles, the textile samples were dried in a Miele T-356 tumble drier, setting ‘Extra dry’. The samples were not ironed. The samples were conditioned overnight at room temperature before testing. The results of spray rating were indicated as SR IN, SR 10L and SR 20L. The results are recorded in Table 13.

TABLE 13
Spray Ratings (1% SOF); Water-based Dispersions
(PES: 76.4 WPU; PA: 74.9% WPU)

| | PES | | | PA | | |
|---|---|---|---|---|---|---|
| Examples | SR IN | SR 10 L | SR 20 L | SR IN | SR 10 L | SR 20 L |
| EX-35 | 100 | 90 | 90 | 100 | 90 | 80 |

These results clearly demonstrate that the fluorine-free treating composition comprising blends of blocked polyurethanes, polycarbodiimides, and polyacrylates provide treated textile substrates, not only with initial high water repellency, but surprisingly also good laundering resistance, after 10 or even after 20 laundering cycles.

The complete disclosures of the patents, patent documents, and publications cited herein are incorporated by reference in their entirety as if each were individually incorporated Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A method of treating a fibrous substrate, the method comprising applying a fluorine-free treating composition in an amount sufficient to make the fibrous substrate water repellent, wherein the treating composition comprises:
one or more polymeric compounds derived from the polymerization of at least one monomer of Formula (I):
R¹-D¹-C(O)C(R²)═CH₂ Formula (I)
wherein:
R¹ represents a hydrocarbon group having 4 to 60 carbon atoms; and
R² represents H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms; and
at least one monomer of Formula (II):
R³-D²-C(O)C(R⁴)═CH₂ Formula (II)
wherein:
R³ represents a hydrocarbon group having 4 to 60 carbon atoms; and
R⁴ represents H or CH₃;
D² is selected from:
—NHC(O)OL¹¹-O—;
O—C(O)NH-L¹²-O—;
—NHC(O)NH-L¹³-O—; and
—O—; and
each L¹¹, L¹², and L¹³ is independently a branched or straight chain alkylene group having 2 to 10 carbon atoms;
wherein the at least one monomer of Formula (II) is present in the one or more polymeric compounds in an amount of less than 30 wt-%, based on the total amount of monomers.

2. (canceled)

3. The method of claim 1 wherein applying the composition to a fibrous substrate comprises applying the composition in an amount sufficient to make the fibrous substrate durably water repellent.

4. A fluorine-free treating composition comprising Component (A) and at least one of Component (B) and Component (C), wherein:
Component (A) comprises one or more polymeric compounds derived from the polymerization of at least one monomer of Formula (I):
R¹-D¹-C(O)C(R²)═CH₂ Formula (I)
wherein:
R¹ represents a hydrocarbon group having 4 to 60 carbon atoms; and
R² represents H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms;
Component (B) comprises:
one or more compounds derived from reaction, in one or more steps, of components comprising:
(i) at least one:
isocyanate-reactive oligomer comprising 2 to 20 repeating units; or
an alcohol, amine, acid, amide, or thiol, having at least one linear or branched hydrocarbon group having from 4 to 60 atoms and optionally one or more ester groups;
(ii) at least one polyisocyanate;
(iii) optionally at least one additional mono-, di-, or poly-functional isocyanate-reactive compound; and
(iv) optionally at least one isocyanate blocking agent;
wherein the isocyanate-reactive oligomer is made by the radical-initiated reaction of at least one (meth)acrylate or (meth)acrylamide monomer, in the presence of at least one mercaptan, wherein the at least one (meth)acrylate or (meth)acrylamide monomer comprises at least one hydrocarbon group having from 4 to 60 carbon atoms; and
Component (C) comprises one or more polycarbodiimide compounds.

5. The fluorine-free treating composition of claim 4 wherein, in Component (A), the one or more polymeric compounds are derived from the polymerization of at least one monomer of Formula (I) and at least one monomer of Formula (II):
R³-D²-C(O)C(R⁴)═CH₂ Formula (II)
wherein:
R³ represents a hydrocarbon group having 4 to 60 carbon atoms; and
R⁴ represents H or CH₃;
D² is selected from:
—NHC(O)OL¹¹-O—;
—O—C(O)NH-L¹²-O—;
—NHC(O)NH-L¹³-O—; and
—O—; and
each L¹¹, L¹², and L¹³ is independently a branched or straight chain alkylene group having 2 to 10 carbon atoms;
wherein the at least one monomer of Formula (II) is present in the one or more polymeric compounds in an amount of less than 30 wt-%, based on the total amount of monomers.

6. The fluorine-free treating composition of claim 4 wherein the components for making the one or more compounds of Component (B) comprise at least one additional mono-, di-, or poly-functional isocyanate-reactive compound.

7. The fluorine-free treating composition of claim 4 wherein the one or more polycarbodiimide compounds of Component (C) are derived from a carbodiimidization reaction, in one or more steps, of components comprising:
(i) at least one:
isocyanate-reactive oligomer comprising 2 to 20 repeating units; or
an alcohol, amine, acid, amide, or thiol, having at least one linear or branched hydrocarbon group having from 4 to 60 carbon atoms and optionally one or more ester groups;
(ii) at least one polyisocyanate; and
(iii) optionally at least one additional mono-, di-, or poly-functional isocyanate-reactive compound;
wherein the isocyanate-reactive oligomer is made by the radical-initiated reaction of at least one (meth)acrylate or (meth)acrylamide monomer, in the presence of at least one mercaptan, wherein the at least one (meth)acrylate or (meth)acrylamide monomer comprises at least one hydrocarbon group having from 4 to 60 carbon atoms.

8. A composition comprising Component (B), wherein:
Component (B) comprises one or more compounds derived from a reaction, carried out in one or more steps, of components comprising:
(i) at least one:
isocyanate-reactive oligomer comprising 2 to 20 repeating units; or
an amine, acid, or amide, having at least one linear or branched hydrocarbon group having from 4 to 60 carbon atoms and optionally one or more ester groups;
(ii) at least one polyisocyanate;
(iii) optionally at least one additional mono-, di- or poly-functional isocyanate-reactive compound; and
(iv) optionally at least one isocyanate blocking agent;
with the proviso that less than 40% of the isocyanate groups are reacted with acid and/or amide groups;
wherein the isocyanate-reactive oligomer has the following formula:
Y¹—[CH₂—C(R²)C(O)-D¹-R¹]ₘ—S—R¹⁰-(T¹)ₚ Formula (V)
wherein:
Y¹ is H or an initiator residue;
R¹ is a hydrocarbon group having 4 to 60 carbon atoms;
R² is independently H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L¹-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the isocyanate-reactive oligomer comprises greater than 30 wt-% of monomeric units that include —NH—, based on the total weight of monomeric units;
each L¹, L², L³, L⁴, L⁵, L⁶, L⁷, L⁸, L⁹, and L¹⁰ is independently a branched or straight chain alkylene group having 2 to 10 carbon atoms;
S is sulfur;
R¹⁰ is a divalent or trivalent linking group having 1 to 10 carbon atoms;
T¹ is —C(O)OH, —C(O)NH₂, —OH, —NH₂, or —NH(R¹¹), wherein R¹¹ is H or a hydrocarbon group having 1 to 10 carbon atoms;
m is an integer of 2 to 20; and
p is independently 1 or 2;
and/or
Component (B) comprises one or more compounds derived from reaction, carried out in one or more steps, of components comprising:
(i) at least one:
a isocyanate-reactive oligomer comprising 2 to 20 repeating units; or
an amine, acid, or amide, having at least one linear or branched hydrocarbon group having from 4 to 60 carbon atoms and optionally one or more ester groups;
(ii) at least one polyisocyanate;
(iii) optionally at least one additional mono-, di- or poly-functional isocyanate-reactive compound; and
(iv) optionally at least one isocyanate blocking agent;
wherein the isocyanate-reactive oligomer has the following formula:
Y²—[CH₂—C(R⁴)C(O)-D²-R³]ₘ—S—R¹²-(T²)ₚ Formula (VI)
wherein:
Y² is H or an initiator residue;
R³ is a hydrocarbon group having 4 to 60 carbon atoms;
R⁴ is independently H or CH₃;
D² is selected from:
—NHC(O)OL¹¹-O—;
—O—C(O)NH-L¹²-O—;
—NHC(O)NH-L¹³-O—; and
—O—; and
each L¹¹, L¹², and L¹³ is independently a branched or straight chain alkylene group having 2 to 10 carbon atoms;
S is sulfur;
R¹² is a divalent or trivalent linking group having 1 to 10 carbon atoms;
T² is —C(O)OH, —C(O)NH₂, —OH, NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
m is an integer of 2 to 20; and
p is independently 1 or 2.

9. The composition of claim 8 further comprising at least one of Component (A) and Component (C), wherein:
Component (A) comprises one or more polymeric compounds derived from the polymerization of at least one monomer comprising at least one hydrocarbon group having from 4 to 60 carbon atoms; and
Component (C) comprises one or more polycarbodiimide compounds.

10. The composition of claim 9 wherein the at least one monomer for making the one or more compounds of Component (A) has at least one of the following formulas:
R¹-D¹-C(O)C(R²)═CH₂ Formula (I)
wherein:
R¹ represents a hydrocarbon group having 4 to 60 carbon atoms; and
R² represents H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms;
R³-D²-C(O)C(R⁴)═CH₂ Formula (II)
wherein:
R³ represents a hydrocarbon group having 4 to 60 carbon atoms; and
R⁴ represents H or CH₃;
D² is selected from:
—NHC(O)OL¹¹-O—;
—O—C(O)NH-L¹²-O—;
—NHC(O)NH-L¹³-O—; and
—O—; and
each L¹¹, L¹², and L¹³ is independently a branched or straight chain alkylene group having 2 to 10 carbon atoms;
R⁵—X¹—C(O)NH-L¹⁴-OC(O)C(R⁶)═CH₂ Formula (III) or
R⁷—X²—C(O)NH-Q¹-NH—C(O)O-L¹⁵-OC(O)C(R⁸)═CH₂ Formula (IV)
wherein:
R⁵ and R⁷ are independently a hydrocarbon group having from 4 to 60 carbon atoms;
R⁶ and R⁸ are independently H or CH₃;
L¹⁴ and L¹⁵ are independently a branched or straight chain alkylene group having 2 to 10 carbon atoms, an arylene group, or a combination thereof,
X¹ is S or —N(R⁹) and X² is O, S, —NH, or —N(R⁹), wherein R⁹ is a hydrocarbon group having 1 to 20 carbon atoms; and
Q¹ is a divalent isocyanate residue.

11. The composition of claim 9 wherein the one or more polycarbodiimide compounds of Component (C) are derived from a carbodiimidization reaction, in one or more steps, of components comprising:
(i) at least one:
isocyanate-reactive oligomer comprising 2 to 20 repeating units; or
an alcohol, amine, acid, amide, or thiol, having at least one linear or branched hydrocarbon group having from 4 to 60 carbon atoms and optionally one or more ester groups;
(ii) at least one polyisocyanate; and
(iii) optionally at least one additional mono-, di-, or poly-functional isocyanate-reactive compound;
wherein the isocyanate-reactive oligomer is made by the radical-initiated reaction of at least one (meth)acrylate or (meth)acrylamide monomer, in the presence of at least one mercaptan, wherein the at least one (meth)acrylate or (meth)acrylamide monomer comprises at least one hydrocarbon group having from 4 to 60 carbon atoms.

12. A composition comprising Component (C), wherein:
Component (C) comprises at least one polycarbodiimide compound derived from a carbodiimidization reaction, carried out in one or more steps, of components comprising:
(i) at least one:
isocyanate-reactive oligomer comprising 2 to 20 repeating units; or
an amine, acid, or amide, having at least one linear or branched hydrocarbon group having from 4 to 60 carbon atoms and optionally one or more ester groups;
(ii) at least one polyisocyanate; and
(iii) optionally at least one additional mono-, di- or poly-functional isocyanate-reactive compound;
wherein the isocyanate-reactive oligomer has the following formula:
Y¹—[CH₂—C(R²)C(O)-D¹-R¹]ₘ—S—R¹⁰-(T¹)ₚ Formula (V)
wherein:
Y¹ is H or an initiator residue;
R¹ is a hydrocarbon group having 4 to 60 carbon atoms;
R² is independently H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O
—NHC(O)NH—; and
—NH—; with the proviso that when D¹ is —NH—, the isocyanate-reactive oligomer comprises greater than 30 wt-% of monomeric units that include —NH—, based on the total weight of monomeric units;
each L¹, L², L³, L⁴, L⁵, L⁶, L⁷, L⁸, L⁹, and L¹⁰ is independently a branched or straight chain alkylene group having 2 to 10 carbon atoms;
S is sulfur;
R¹⁰ is a divalent or trivalent linking group having 1 to 10 carbon atoms;
T¹ is —C(O)OH, —C(O)NH₂, —OH, —NH₂, or —NH(R¹¹), wherein R¹¹ is H or a hydrocarbon group having 1 to 10 carbon atoms;
m is an integer of 2 to 20; and
p is independently 1 or 2;
and/or
Component (C) comprises at least one polycarbodiimide compound derived from a carbodiimidization reaction, carried out in one or more steps, of components comprising:
(i) at least one:
isocyanate-reactive oligomer comprising 2 to 20 repeating units; or
an amine, acid, or amide, having at least one linear or branched hydrocarbon group having from 4 to 60 carbon atoms and optionally one or more ester groups;
(ii) at least one polyisocyanate; and
(iii) optionally at least one additional mono-, di- or poly-functional isocyanate-reactive compound;
wherein the isocyanate-reactive oligomer has the following formula:
Y²—[CH₂—C(R⁴)C(O)-D²-R³]ₘ—S—R¹²-(T²)ₚ Formula (VI)
wherein:
Y² is H or an initiator residue;
R³ is a hydrocarbon group having 4 to 60 carbon atoms;
R⁴ is independently H or CH₃;
D² is selected from:
—NHC(O)OL¹¹-O—;
—O—C(O)NH-L¹²-O—;
—NHC(O)NH-L¹³-O—; and
—O—; and
each L¹¹, L¹², and L¹³ is independently a branched or straight chain alkylene group having 2 to 10 carbon atoms;
S is sulfur;
R⁷ is a divalent or trivalent linking group having 1 to 10 carbon atoms;
T² is —C(O)OH, —C(O)NH₂, —OH, NH₂ or —NH(R¹¹), wherein R¹¹ is H or a hydrocarbon group having 1 to 10 carbon atoms; with the proviso that when T² is —OH, —NH₂ or —NH(R¹¹), component (iii) is present and includes an acid or an amide functionality;
m is an integer of 2 to 20; and
p is independently 1 or 2;
wherein the composition is fluorine-free.

13. The composition of claim 12 further comprising at least one of Component (A) and Component (B), wherein:
Component (A) comprises one or more polymeric compounds derived from the polymerization of at least one monomer comprising at least one hydrocarbon group having from 4 to 60 carbon atoms;
and
Component (B) comprises:
one or more compounds derived from reaction, in one or more steps, of components comprising:
(i) at least one:
isocyanate-reactive oligomer comprising 2 to 20 repeating units; or
an alcohol, amine, acid, amide, or thiol, having at least one linear or branched hydrocarbon group having from 4 to 60 carbon atoms and optionally one or more ester groups;
(ii) at least one polyisocyanate;
(iii) optionally at least one additional mono-, di-, or poly-functional isocyanate-reactive compound; and
(iv) optionally at least one isocyanate blocking agent;
wherein the isocyanate-reactive oligomer is made by the radical-initiated reaction of at least one (meth)acrylate or (meth)acrylamide monomer, in the presence of at least one mercaptan (which may or may not be functionalized), wherein the at least one (meth)acrylate or (meth)acrylamide monomer comprises at least one hydrocarbon group having from 4 to 60 carbon atoms.

14. The composition of claim 13 wherein the at least one monomer for making the one or more compounds of Component (A) has at least one of the following formulas:
R¹-D¹-C(O)C(R²)═CH₂ Formula (I)
wherein:
R¹ represents a hydrocarbon group having 4 to 60 carbon atoms; and
R² represents H or CH₃;
D¹ is selected from:
—C(O)O-L¹-O—;
—OC(O)-L²-O—;
—OC(O)-L³-C(O)O-L⁴-O—;
—C(O)NH-L⁵-O—;
—NHC(O)-L⁶-O—;
—NHC(O)-L⁷-C(O)O-L⁸-O—;
—C(O)—NH—;
—C(O)NHC(O)NH-L⁹-O—;
—C(O)NHC(O)-L¹⁰-O—;
—NHC(O)NH—; and
—NH—; with the proviso that when D¹ is —NH—, the one or more polymeric compounds are derived from greater than 30 wt-% of monomers of Formula (I), based on the total weight of monomers; and
each L¹, L², L³, L⁴, L⁵, L⁶, L⁷, L⁸, L⁹, and L¹⁰ is independently a branched or straight chain alkylene group having 2 to 10 carbon atoms;
R³-D²-C(O)C(R⁴)═CH₂ Formula (II)
wherein:
R³ represents a hydrocarbon group having 4 to 60 carbon atoms; and
R⁴ represents H or CH₃;
D² is selected from:
—NHC(O)OL¹¹-O—;
—O—C(O)NH-L¹²-O—;
—NHC(O)NH-L¹³-O—; and
—O—; and
each L¹¹, L¹², and L¹³ is independently a branched or straight chain alkylene) having 2 to 10 carbon atoms;
R⁵—X¹—C(O)NH-L¹⁴-OC(O)C(R⁶)═CH₂ Formula (III) or
R⁷—X²—C(O)NH-Q¹-NH—C(O)O-L¹⁵-OC(O)C(R⁸)═CH₂ Formula (IV)
wherein:
R⁵ and R⁷ are independently a hydrocarbon group having from 4 to 60 carbon atoms;
R⁶ and R⁸ are independently H or CH₃;
L¹⁴ and L¹⁵ are independently a branched or straight chain alkylene group having 2 to 10 carbon atoms, an arylene group, or a combination thereof,
X¹ is S or —N(R⁹) and X² is O, S, —NH, or —N(R⁹), wherein R⁹ is a hydrocarbon group having 1 to 20 carbon atoms; and
Q¹ is a divalent isocyanate residue.

15. The composition of claim 13 wherein the components for making the one or more compounds of Component (B) comprise at least one additional mono-, di-, or poly-functional isocyanate reactive compound.

16. The composition of claim 13 wherein, in Component (B), less than 40% of the isocyanate groups are reacted with acid and/or amide groups.

17. The composition of claim 8 which is a fluorine-free treating composition.

18. A method of treating a fibrous substrate, the method comprising applying a composition of any one of claims 4 through 17 in an amount sufficient to make the fibrous substrate water repellent.

19. The method of claim 18 wherein applying the composition to a fibrous substrate comprises applying the composition in an amount sufficient to make the fibrous substrate durably water repellent.

20. A fibrous substrate treated by the method of any one of claims 1, 3 , 18 or 19.
